(19) United States
(12) Patent Application Publication
(10) Pub. No.: US 2021/0246519 A1
(43) Pub. Date: Aug. 12, 2021

(54) SPIKED PRIMERS FOR ENRICHMENT OF PATHOGEN NUCLEIC ACIDS AMONG BACKGROUND OF NUCLEIC ACIDS

(71) Applicant: The Regents of the University of California, Oakland, CA (US)

(72) Inventors: Charles Chiu, Oakland, CA (US); Xianding Deng, Oakland, CA (US)

(21) Appl. No.: 17/049,320

(22) PCT Filed: May 3, 2019
(86) PCT No.: PCT/US2019/030746

§ 371 Date: Oct. 20, 2020

Related U.S. Application Data
(60) Provisional application No. 62/667,334, filed on May 4, 2018.
(60) Provisional application No. 62/816,003, filed on Mar. 8, 2019.

(51) Int. Cl. C12Q 1/70; C12Q 1/6869; C12Q 1/6895; G16B 25/20; G16B 10/00; G16B 30/10

(52) U.S. Cl. CPC C12Q 1/703; C12Q 1/6869; G16B 30/10; G16B 25/20; G16B 10/00; C12Q 1/6895

(57) ABSTRACT

Methods, compositions and kits for detection of a taxon of pathogenic microorganisms in a sample are provided. Also provided are methods, compositions and kits for detecting taxons of pathogenic microorganisms present in low titers in a sample. Compositions, methods and kits for detection of pathogenic co-infections are also provided.

### CROSS REFERENCE TO RELATED APPLICATIONS

This application claims priority under 35 U.S.C. § 119 from Provisional Application Ser. No. 62/667,334 filed May 4, 2018, and from Provisional Application Ser. No. 62/816,003 filed Mar. 8, 2019, the disclosures of which are incorporated herein by reference for all purposes.

### GOVERNMENT LICENSE RIGHTS

This invention was made with Government support under Grant Nos: R21 AI120977 and R01 HL105704, awarded by the National Institutes of Health. The Government has certain rights in the invention.

### FIELD

The present disclosure relates to the field of genomics and diagnostics, and more particularly to the detection and genomic characterization of pathogenic microorganisms in a sample.

### INCORPORATION BY REFERENCE OF SEQUENCE LISTING

Accompanying this filing is a Sequence Listing entitled “00138-005WO1_SL.txt”, created on Apr. 30, 2019, and having 1,706,058 bytes of data, machine formatted on IBM-PC, MS-Windows operating system. The sequence listing is hereby incorporated herein by reference in its entirety for all purposes.

### BACKGROUND

The threat from new or re-emerging viruses has markedly increased in recent decades due to population growth, urbanization, and expansion of global travel, facilitating rapid spread of infection during an outbreak. Over the past 4 decades, epidemics from human immunodeficiency virus (HIV) (1981-present), SARS (2002-2004) and MERS (2012-present) coronaviruses, 2009 pandemic influenza H1N1 and avian influenza viruses (1996-present), EBOV virus (EBOV) in West Africa (2013-2016) and Zika virus (ZIKV) in the Americas (2015-2016) (Reperant and Osterhaus, 2017) have occurred. Initial identification and containment of these outbreaks were hindered by their occurrence in resource-poor settings and/or the lack of access to diagnostic assays that could detect a novel, unanticipated viral strain. This lack of preparedness underscores the important need for the deployment of effective diagnostic and surveillance tools able to rapidly screen for infected patients and guide public health interventions that curb transmission.

### SUMMARY

Various embodiments of the disclosure address the above problems of pathogen detection and enrichment. For example, a method of detecting a first taxon of pathogenic microorganisms in a sample can include: obtaining an environmental, plant, or animal sample and/or a sample from a human subject to be screened for a first taxon of pathogens, applying a sequencing assay to the sample to obtain sequence reads, and determining whether the first taxon of pathogenic microorganisms is present in the sample. The sequencing assay includes primers (e.g., spiked primers) that are of such a length (e.g., 11-17 base pairs) as to preferentially amplify certain sequences associated with the first taxon and that do not amplify sequences associated with one or more other taxa of pathogens. Generally, the one or more other taxa of pathogenic microorganisms are amplified using random primers, present in an amount, proportion or ratio that is less than the primers associated with the first taxon of pathogenic microorganisms (e.g., spiked primers). Generally, the ratio of spiked primers to random primer is about 5:1, about 10:1, about 20:1, or more. In this manner, if the sample (environmental, plant, animal, and/or from a human subject) is possibly infected with, or has been exposed to one or more pathogenic microorganisms (including the first taxon of pathogens), embodiments allow for the screening of multiple pathogenic microorganisms using the same assay, while still allowing certain pathogenic microorganisms to be targeted so as to enable detection when present at low amounts. Additionally, the methods provide for the enrichment of the first taxon through the use of spiked primers without sacrificing metagenomic sensitivity obtained utilizing random primers. Thus, detection of untargeted pathogenic microorganisms and/or pathogenic co-infections within the sample can be enabled by detecting two different pathogen taxa from the same sample.

Advantages of the disclosure over multiplex PCR and probe capture techniques include a lack of a requirement for primer optimization, which is typically required in multiplex PCR, and improved detection times, as compared to probe capture techniques that usually require an additional >18-24-hour period due to hybridization times. Another advantageous attribute of the disclosure is that the methods provide sufficient enrichment of a first taxon of pathogenic microorganisms that allows for detection of the first taxon of pathogenic microorganisms that is less affected by the background of the host organism (e.g., human) nor hinders the detection of other pathogen taxa present in the sample. For example, the use of smaller proportions of random primers as compared to spiked primers resulted in less amplification of high-background sequences in a sample (e.g., human ribosomal RNA, etc.) and thus enriched the sample for pathogenic microorganisms by depleting the presence of human host sequences in the sample sequencing library. The method therefore provides adequate enrichment of a first taxon of pathogenic microorganisms while not adversely affecting metagenomic sensitivity.

In one example, the sequencing assay can include primers having a length within a range of 11-17 base pairs, wherein at least a portion of the sequence regions targeted by the primers were identified in a first set or one or more reference sequences corresponding to the first taxon of pathogens. In one example, the sequencing assay can include a plurality of primers wherein at least one primer from SEQ ID NOs: 1-96, 399-1562, 1563-3553, and 3554-7324.

In another example, the sequencing assay can include a polymerase chain reaction (PCR) or any of its derivatives such as, but not limited to, real-time PCR, quantitative PCR, reverse transcription PCR, and reverse transcription quantitative PCR, or a non-PCR amplification strategy such as isothermal transcription-mediated amplification (TMA). The sequencing assay can comprise from 1 to 5,000 or more primer pairs. In one example, the sequencing assay can include reverse transcription of a sample containing RNA using any of the primers disclosed herein. In another example, any of the primers in SEQ ID NOs: 1-96, 399-1562, 1563-3553, and 3554-7324 can be included in the sequencing assay. In one example, the primers can further comprise a nucleic acid adapter sequence. For example, when performing reverse transcription PCR (RT-PCR) using the methods of the disclosure for identifying RNA viruses, the presence of an adapter is optional; however, when performing the methods of the disclosure on an DNA sample (e.g., bacterial genomes), an adapter can be particularly useful. In one embodiment, an adapter can be attached to any of the primers in SEQ ID NOs: 3554-7324 at the 5′ end. Preferably, the nucleic acid adapter is located 5′ of the primer. In one example, the adapter is used as a primer in a subsequent nucleic acid amplification reaction (e.g., PCR). The adapter sequence can also have embedded within it type I and type IIs restriction endonucleases sites such that, when cleaved by restriction endonucleases, they produce overhangs (staggered ends) that can be selectively ligated with another adapter for next-generation sequencing (NGS). In some embodiments, the adapter comprises SEQ ID NO:97. The sequencing reads obtained by the sequencing assay may be present in raw or processed form to remove, for example, low quality or low-complexity sequencing reads. In one example, the sequencing assay provides greater than 10 sequencing reads and fewer than 100,000 sequencing reads per amplified target nucleic acid present in the sample.

The disclosure also provides an oligonucleotide comprising, consisting essentially of or consisting of any one or more of the sequences set forth in SEQ ID NOs: 1-96, 399-1562, 1563-3553, and/or 3554-7324 or probes set forth in SEQ ID NO:98-397 and/or 398.

The sequencing assay can further include one or more probes, such as but not limited to, any of the probes set forth in SEQ ID NOs: 98-398. In some embodiments, the probes are labeled or produce a detectable signal from which a user can determine whether the first taxon of pathogenic microorganisms is present in the sample. In one example, the probe may be used to determine (e.g., quantify) the amount of amplified target produced by the primers of the sequencing assay.

In some embodiments, the first taxon of pathogenic microorganisms is selected from bacteria, archaea, viruses, protozoa, prions, fungi, algae, microscopic parasites (e.g., helminths) or other disease- or illness-inducing microbe. In some embodiments, the first taxon of pathogenic microorganisms corresponds to viral pathogens, such as but not limited to viruses of List 1. In one example, the first taxon is a Flavivirus or Alphavirus. In another example, the first taxon of pathogenic microorganisms encompasses one or more species selected from West Nile Virus, dengue virus, tick-borne encephalitis virus, Chikungunya virus, Ebola virus, Marburg virus, Lassa virus, Rift Valley Fever Virus, Crimean-Congo hemorrhagic fever virus, Japanese encephalitis virus, yellow fever virus, Zika virus, cell fusing agent virus, Palm Creek virus and Parramatta River virus. Of particular interest are viruses present in the sample in low viral titers (i.e., less than 10,000 viral genome copies per mL of sample). For example, the first taxon of pathogenic microorganisms can be present in the sample at a volume of less than 10,000 genome copies per mL, less than 9,000 genome copies per mL, less than 8,000 genome copies per mL, less than 7,000 genome copies per mL, less than 6,000 genome copies per mL, less than 5,000 genome copies per mL, less than 4,000 genome copies per mL, less than 3,000 genome copies per mL, less than 2,000 genome copies per mL, less than 1,000 genome copies per mL, less than 900 genome copies per mL, less than 800 genome copies per mL, less than 700 genome copies per mL, less than 600 genome copies per mL, less than 500 genome copies per mL, less than 400 genome copies per mL, less than 300 genome copies per mL, less than 200 genome copies per mL, less than 100 genome copies per mL, less than 90 genome copies per mL, less than 80 genome copies per mL, less than 70 genome copies per mL, less than 60 genome copies per mL, less than 50 genome copies per mL, less than 40 genome copies per mL, less than 30 genome copies per mL, less than 20 genome copies per mL, less than 10 genome copies per mL, or any range of genome copies per mL that includes or is between and two of the foregoing genome copies per mL (e.g., from 100 genome copies per mL to 1,000 genome copies per mL). Accordingly, the methods, kits and compositions of the disclosure allow for detection of a co-infection in the sample.

The methods, compositions and kits disclosed herein can be used with a variety of sample types from a variety of different sources (e.g., clinical, plant, animal, or environmental samples). For example, the sample can include whole blood, serum, plasma, urine, tissue samples, biopsy samples, water samples, food samples, environmental samples, test-wipes of a location or device, and isolated nucleic acids. In one embodiment, the sample is obtained from a human subject, such as a human patient or a subject believed to be infected by a first taxon of pathogens. In another embodiment, the sample is obtained from an environmental site believed to be infected or contaminated by a first taxon of pathogens. Environmental sites, can include sites found outdoors or indoors, such as a hospital room.

In some embodiments, the one or more reference sequences corresponding to the first taxon of pathogenic microorganisms can comprise a complete or partial genome of the first taxon of pathogens.

Multiple reference genomes or portions of the genome (such as individual genes) can be used for spiked primer design, including those from different phyla (e.g., viral, fungal, or bacterial genomes). A multiple sequence alignment can be made of a number of related genomes (from 2 to 10,000 and any integer therebetween) and either the consensus alignment sequence or the totality of the aligned sequences can be used as the reference to design the spiked primers (FIGS. 2A and 10A). The disclosure provides a method for providing and developing a spiked primer composition. The method includes aligning 2 to 10,000 related genomes (e.g., viral, bacterial, or fungi genomes). By “related genomes” means genomes in the same taxonomic phylum, class, order family or genus. Identifying, from the alignment, overlapping sequences having a length of 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, or any range that includes or is between any two of the foregoing values, (e.g., 50-100 bp, 50-200 bp, 50-300 bp, 50-500 bp, 50-1000 bp, 100-200 bp, 100-300 bp, 100-400 bp, 100-500 bp, 200-300 bp, 200-400 bp, 200-500 bp, 300-400 bp, and 300-500 bp in length (and any integer between any of the foregoing ranges). Determining a primer pair (forward and reverse) that are about 10-50 bp in length from the overlapping sequence. For each primer sequence identifying the shortest unique primer length (i.e., the k-mer) which is typically about 11-17 nt in length (e.g., about 13 nt in length). Filter the selected primers by Tm (e.g., <2 SD from mean) and remove self-dimers or cross-dimers and remove homopolymer repeats (e.g., >5 nt). Providing a library of the identified primers based upon the foregoing. In one embodiment, the method further includes generating a plurality of the primers so identified using an oligonucleotide synthesizer and assaying the “spiked” primers generated.

In another example, a nucleic acid molecule for detecting a target sequence from a first taxon of pathogenic microorganisms is disclosed. The nucleic acid molecule can include a primer that is at least 90% complementary to the target sequence. In one embodiment, the primer is 10, 11, 12, 13, 14, 15, 16, 17, 18, 19 or 20 nucleotides in length, or any range that includes or is between any two of the foregoing values (e.g., 11 to 17 nucleotides in length). In another embodiment, the primer consists of one of the sequences in SEQ ID NOs: 1-96, 399-1562, 1563-3553, and 3554-7324 alone or wherein the primer consists of the “primer” sequence and is linked to an adapter sequence. For example, primers that are complementary or substantially complementary to the genome of one or more human viruses are contemplated by the disclosure. In some embodiments, the primer molecule includes a nucleic acid adapter positioned 5′ of the primer. In one example, the adapter comprises SEQ ID NO:97.

In another example, the present disclosure provides a kit comprising at least one primer set forth in SEQ ID NOs: 1-96, 399-1562, 1563-3553, and 3554-7324. In some embodiments, the kit further comprises an adapter appended to the 5′ terminus of a primer for use in a sequencing assay. In one example, the adapter is SEQ ID NO:97, other adapters useful for various sequencing methods will be readily identified by one of skill in the art. In some embodiments, the kit further comprises one or more additional primers, probes or reagents. In one example, the one or more additional primers can include primers that are between 6 and 12 nucleotides in length. In one example, the additional primers can include one or more nucleotide modifications. In another example, the additional primers include random hexamers, random septamers, random octamers, or random nonamers. In one example, the kit can further include one or more probes as set forth in SEQ ID NO: 98-398.

In a particular embodiment, the disclosure provides a method for designing spiked primer sequences that enrich sequencing reads for detecting a taxon or taxa of pathogenic microorganisms in a sample, the method comprising: (i) performing multiple sequence alignments (MSA) of a plurality of genomes from a set of one or more reference genomes from a taxon or taxa of pathogens; (ii) partitioning the MSA-aligned genomes into overlapping 300 to 600 nucleotide (nt) segments with at least a 150 nt overlap; (iii) selecting forward and/or reverse candidate primer sequences having lengths that are within a range of 11 bp to 17 bp from 30 to 70 nt regions at the ends of each 300 to 600 nt segment by frequency of occurrence in the set of overlapping 300 to 600 nt segments: (iv) ranking the candidate primer sequences iteratively in reverse order by frequency of occurrence in the overlapping 300 to 600 nucleotide (nt) segments; (v) selecting top candidate primer sequences based upon the candidate primer sequences being shared by the most 300 to 600 nt segments and by not containing any ambiguous 300 to 600 nt segments; (vi) removing 300 to 600 nt segments which share top candidate primer sequences and repeating steps (iii) to (v) until the number of the remaining 300 to 600 nt segments containing a shared candidate primer sequence is below a pre-designated threshold integer value selected from 1 to 15 in order to generate a set of top candidate primer sequences; (viii) generating a set of spiked primer sequences from the set of top candidate primer sequences by removing top candidate primer sequences that (a) have melting temperatures (Tm) greater than 2 standard deviations from the mean, (b) are predicted to self-dimerize or cross dimerize with a hybridization ΔG<−9 kcal/mol or lower, and/or (c) have homopolymer repeats of greater than 5 nucleotides. In another embodiment, the set of one or more reference genomes are from a taxon or taxa of pathogenic bacteria, viruses, protozoa, fungi, archaea, algae and/or eukaryotic parasites. In yet another embodiment, the set of one or more reference genomes encompasses viral genomes selected from a taxon or taxa of one or more Families of viruses including Reoviridae, Caliciviridae, Flaviviridae, Orthomyxoviridae, Picornaviridae, Togaviridae, Paramyxoviridae, Bunyaviridae, Rhabdoviridae, Filoviridae, Coronaviridae, Astroviridae, Bornaviridae, Arteriviridae, Hepeviridae, and/or Retroviridae. In a further embodiment, the set of one or more reference genomes encompasses viral genomes selected from a taxon of the Flaviviridae Family of viruses. In yet a further embodiment, the set of one or more reference genomes encompasses viral genomes selected from a taxon or taxa of one or more Genera of viruses including Ahjdlikevirus, Alfamovirus, Allexivirus, Allolevivirus, Alphabaculovirus, Alphacarmotetravirus, Alphacoronavirus, Alphaentomopoxvirus, Alphafusellovirus, Alphaguttavirus, Alphalipothrixvirus, Alphamesonivirus, Alphanecrovirus, Alphanodavirus, Alphanudivirus, Alphapapillomavirud, Alphapartitivirus, Alphapermutotetravirus, Alpharetrovirus, Alphasphaerolipovirus, Alphaspiravirus, Alphatorquevirus, Alphaturrivirus, Alphavirus, Amalgavirus, Ambidensovirus Amdoparvovirus, Ampelovirus, Ampullavirus, Andromedalikevirus, Anulavirus, Aparavirus, Aphthovirus, Apscaviroid, Aquabirnavirus, Aquamavirus, Aquaparamyxovirus, Aquareovirus, Arterivirus, Ascovirus, Asfivirus, Atadenovirus, Aureusvirus, Aurivirus Avastrovirus, Avenavirus, Aveparvovirus, Aviadenovirus, Avibirnavirus, Avihepadnavirus, Avihepatovirus, Avipoxvirus, Avisivirus, Avsunviroid, Avulavirus, Bacillarnavirus, Babuvirus, Bacilladnavirus, Barnavirus, Badnavirus, Bafinivirus, Bcep22likevirus, Barnyardlikevirus, Batrachovirus, Bdellomicrovirus, Bcep78likevirus, Bcepmulikevirus, Benyvirus, Becurtovirus, Begomovirus, Betaentomopoxvirus, Betabaculovirus, Betacoronavirus, Betalipothrixvirus, Betafusellovirus, Betaguttavirus, Betanudivirus, Betanecrovirus, Betanodavirus, Betaretrovirus, Betapapillomavirus, Betapartitivirus, Betatorquevirus, Betasphaerolipovirus, Betatetravirus, Bignuzlikevirus, Bicaudavirus, Bidensovirus, Bornavirus, Blosnavirus, Bocaparvovirus, Bracovirus, Botrexvirus, Bppunalikevirus, Bromovirus, Brambyvirus, Brevidensovirus, Bronlikevirus, Cafeteriavirus, Bymovirus, Cardiovirus, C2likevirus, C5likevirus, Carmovirus, Capillovirus, Capripoxvirus, Cervidpoxvirus, Cardoreovirus, Carlavirus, Che9clikevirus, Caulimovirus, Cavemovirus, Chipapillomavirus, Charlielikevirus, Che8likevirus, Chlorovirus, Cheravirus, Chilikevirus, Circovirus, Chlamydiamicrovirus, Chloriridovirus, Clavavirus, Chrysovirus, Cilevirus, Coccolithovirus, Citrivirus, Cjwunalikevirus, Comovirus, Closterovirus, Cocadviroid, Corticovirus, Coleviroid, Coltivirus, Cp8unalikevirus, Copiparvovirus, Corndoglikevirus, Crocodylidpoxvirus, Cosavirus, Cp220likevirus, Cuevavirus, Crinivirus, Cripavirus, Cyprinivirus, Cryspovirus, Cucumovirus, Cytorhabdovirus, Curtovirus, Cypovirus, Cystovirus, Cytomegalovirus, D3likevirus, Deltalipothrixvirus, Deltabaculovirus, D3112likevirus, Deltaretrovirus, Deltapapillomavirus, Deltacoronavirus, Dependoparvovirus, Deltatorquevirus, Deltapartitivirus, Dinodnavirus, Dianthovirus, Deltavirus, Dyodeltapapillomavirus, Dinornavirus, Dicipivirus, Dyoiotapapillomavirus, Dyoepsilonpapillomavirus, Dinovernavirus, Dyomupapillomavirus, Dyokappapapillomavirus, Dyoetapapillomavirus, Dyopipapillomavirus, Dyonupapillomavirus, Dyolambdapapillomavirus, Dyothetapapillomavirus Dyorhopapillomavirus, Dyoomikronpapillomavirus, Dyoxipapillomavirus, Dyosigmapapillomavirus, Dyozetapapillomavirus, Emaravirus, Enterovirus, Ebolavirus, Epsilon15likevirus, Enamovirus, Elaviroid, Epsilontorquevirus, Entomobirnavirus, Endornavirus, Errantivirus, Epsilonpapillomavirus, Ephemerovirus, Etatorquevirus, Eragrovirus, Epsilonretrovirus, Fabavirus, Erythroparvovirus, Erbovirus, Fijivirus, Felixounalikevirus, Etapapillomavirus, Furovirus, Flavivirus, F116likevirus, Ferlavirus, Foveavirus, Gallivirus, Gammabaculovirus, Gammaentomopoxvirus, Gammalipothrixvirus, Gammapartitivirus, Gammaretrovirus, Gallantivirus, Gammatorquevirus, Giardiavirus, Gammacoronavirus, Glossinavirus, Gyrovirus, Gammapapillomavirus, Gammasphaerolipovirus, Globulovirus, Halolikevirus, Hantavirus, Hemivirus, Hempavirus, Hepandensovirus, Hepatovirus, Hapunalikevirus, Hk578likevirus, Hordeivirus, Hepacivirus, Hpunalikevirus, Hunnivirus, Higrevirus, Hostuviroid, Hypovirus, Ichtadenovirus, I3likevirus, Idnoreovirus, Ictalurivirus, Ilarvirus, Iebhlikevirus, Ichnovirus, Influenzavirus B, Iltovirus, Idaeovirus, Iotapapillomavirus, Influenzavirus C, Iflavirus, Iridovirus, Iotatorquevirus, Influenzavirus A, Inovirus, Ipomovirus, Isavirus, Iteradensovirus, Jerseylikevirus, Kappapapillomavirus, Kappatorquevirus, Kobuvirus, Kunsagivirus, Labyrnavirus, Lambdapapillomavirus, Lagovirus, Lentivirus, Lambdatorquevirus, L5likevirus, Lolavirus, Leporipoxvirus, Lambdalikevirus, Lymphocryptovirus, Luteovirus, Leishmaniavirus, Lymphocystivirus, Levivirus, Luz24likevirus, Lyssavirus, Machlomovirus, Mamastrovirus, Macanavirus, Marafivirus, Macluravirus, Marnavirus, Mammarenavirus, Macavirus, Mastrevirus, Marburgvirus, Maculavirus, Megrivirus, Marseillevirus, Mandarivirus, Microvirus, Megabirnavirus, Mardivirus, Mischivirus, Metapneumovirus, Mastadenovirus, Morbillivirus, Mimivirus, Megalocytivirus, Mupapillomavirus, Mitovirus, Metavirus, Mycoflexivirus, Mosavirus, Mimoreovirus, Muromegalovirus, Molluscipoxvirus, Mycoreovirus, Mulikevirus, Muscavirus, N15likevirus, Nanovirus, Nepovirus, N4likevirus, Nucleorhabdovirus, Narnavirus, Nairovirus, Norovirus, Nebovirus, Nupapillomavirus, Novirhabdovirus, Nyavirus, Omegalikevirus, Omikronpapillomavirus, Orthobunyavirus, Okavirus, Orthopoxvirus, Omegapapillomavirus, Oleavirus, Oscivirus, Ophiovirus, Omegatetravirus, Orthohepadnavirus, Orbivirus, Orthoreovirus, Orthohepevirus, Ostreavirus, Oryzavirus, Ourmiavirus, Parechovirus, Pbiunalikevirus, Pegivirus, P2likevirus, P22likevirus, Percavirus, Panicovirus, P23likevirus, Petuvirus, Pasivirus, Parapoxvirus, Phi29likevirus, Pbunalikevirus, Passerivirus, Phicd119likevirus, Pelamoviroid, Pecluvirus, Phietalikevirus, Perhabdovirus, Penstyldensovirus, Phijlunalikevirus, Pgonelikevirus, Pestivirus, Phipapillomavirus, Phic3unalikevirus, Phaeovirus, Picobirnavirus, Phie125likevirus, Phicbklikevirus, Plasmavirus, Phyllikevirus, Phieco32likevirus, Poacevirus, Phikmvlikevirus, Phihlikevirus, Polyomavirus, Phlebovirus, Phikzlikevirus, Potexvirus, Pipapillomavirus, Phytoreovirus, Proboscivirus, Plectrovirus, Piscihepevirus, Pseudovirus, Polemovirus, Pneumovirus, Punalikevirus, Pomovirus, Polerovirus, Potyvirus, Pospiviroid, Protoparvovirus, Prasinovirus, Prymnesiovirus, Psimunalikevirus, Psipapillomavirus, Quaranjavirus, Quadrivirus, Raphidovirus, Reylikevirus, Rhopapillomavirus, Reptarenavirus, Roseolovirus, Rhadinovirus, Rubulavirus, Rosadnavirus, Ranavirus, Rotavirus, Respirovirus, Rubivirus, Rudivirus, Rymovirus, Rhizidiovirus, Rosavirus, Salivirus, Sap6likevirus, Schizot4virus, Sadwavirus, Seadornavirus, Salmonivirus, Sequivirus, Sapelovirus, Siadenovirus, Sclerodarnavirus, Sakobuvirus, Sigmavirus, Semotivirus, Salterprovirus, Skunalikevirus, Sfi1unalikevirus, Sapovirus, Soymovirus, Sicinivirus, Scutavirus, Spiromicrovirus, Simplexvirus, Senecavirus, Spumavirus, Sobemovirus, Sfi21dtunalikevirus, Sp6likevirus, Sigmapapillomavirus, Spounalikevirus, Sirevirus, Suipoxvirus, Solendovirus, Spbetalikevirus, Sprivivirus, T4virus, Taupapillomavirus, Tepovirus, Thetapapillomavirus, T5likevirus, Tibrovirus, Tectivirus, Tobravirus, Teschovirus, T7likevirus, Torovirus, Thetatorquevirus, Tenuivirus, Totivirus, Tm4likevirus, Tetraparvovirus, Trichomonasvirus, Tombusvirus, Thogotovirus, Tunalikevirus, Torradovirus, Tobamovirus, Turncurtovirus, Tp2unalikevirus, Topocuvirus, Trichovirus, Tospovirus, Tungrovirus, Tremovirus, Twortlikevirus, Tritimovirus, Tupavirus, Tymovirus, Umbravirus, Upsilonpapillomavirus, Varicellovirus, Velarivirus, Vesiculovirus, Victorivirus, Vitivirus, Varicosavirus, Vesivirus, Viunalikevirus, Waikavirus, Wbetalikevirus, Whispovirus, Xp10likevirus, Xipapillomavirus, Yualikevirus, Yatapoxvirus, Zetapapillomavirus, Zetatorquevirus, and/or Zeavirus. In a certain embodiment, the set of one or more reference genomes encompasses viral genomes from a taxon or taxa of one or more Species of viruses including West Nile Virus, dengue virus, tick-borne encephalitis virus, Japanese encephalitis virus, yellow fever virus, Zika virus, cell fusing agent virus, Palm Creek virus and/or Parramatta River virus. In another embodiment, the set of one or more reference genomes encompasses bacterial genomes from one or more from a taxon or taxa of one or more Genera of bacteria including Heliobacter, Aerobacter, Rhizobium, Agrobacterium, Bacillus, Clostridium, Pseudomonas, Xanthomonas, Nitrobacteriaceae, Nitrobacter, Nitrosomonas, Thiobacillus, Spirillum, Vibrio, Bacteroides, Corynebacterium, Listeria, Escherichia, Klebsiella, Salmonella, Serratia, Shigella, Erwinia, Rickettsia, Chlamydia, Mycoplasma, Actinomyces, Streptomyces, Mycobacterium, Polyangium, Micrococcus, Staphylococcus, Lactobacillus, Diplococcus, Streptococcus, and/or Campylobacter. In yet another embodiment, the set of one or more reference genomes encompasses fungal genomes from one or more from a taxon or taxa of one or more Genera of fungi including Anaeromyces, Caecomyces, Allomyces, Entyloma, Diskagma, Blastocladia, Funneliformis, Entylomella, Coelomomyces, Glomus (fungus), Fusidium, Heptameria, Holmiella, Homostegia, Hyalocrea, Hyalosphaera, Hypholoma, Hypobryon, Hysteropsis, Koordersiella, Karschia, Kirschsteiniothelia, Lembosiopeltis, Kullhemia, Kusanobotrys, Leptodothiorella, Lanatosphaera, Lasiodiplodia, Leveillina, Lepidopterella, Lepidostroma, Lollipopaia, Leptosphaerulina, Leptospora, Macrovalsaria, Lichenostigma, Licopolia, Massariola, Lopholeptosphaeria, Maireella, Microdothella, Macroventuria, Microcyclella, Mycoglaena, Melanodothis, Montagnella, Mycoporopsis, Moniliella, Mycopepon, Myriangium, Mycomicrothelia, Mycothyridium, Mytilostoma, Mycosphaerella, Mytilinidion, Neofusicoccum, Myriostigmella, Neocallimastix, Oomyces, Neopeckia, Orpinomyces, Ostreichnion, Ophiosphaerella, Paropodia, Passeriniella, Passerinula, Pedumispora, Peyronellaea, Phaeoacremonium, Phaeocyrtidula, Phaeoglaena, Phaeopeltosphaeria, Phaeoramularia, Phaeosperma, Phaneromyces, Phialophora, Philonectria, Phragmocapnias, Phragmosperma, Piedraia, Piromyces, Placocrea, Placostromella, Plagiostromella, Plejobolus, Pleostigma, Polychaeton, Pseudocercospora, Pseudocryptosporella, Pseudogymnoascus, Pseudothis, Pycnocarpon, Rhytidhysteron, Rhizophagus (fungus), Rhopographus, Rosellinula, Rhytisma, Robillardiella, Roussoellopsis, Rosenscheldia, Rostafinskia, Sarcopodium, Savulescua, Saksenaeaceae, Scolecobonaria, Scolicotrichum, Schizoparme, Semifissispora, Septoria, Scorias, Sphaceloma, Sphaerellothecium, Spathularia, Stagonosporopsis, Stenella (fungus), Sphaerulina, Stigmina (fungus), Stioclettia, Stigmidium, Sydowia, Tephromela, Stuartella, Teichosporella, Thalloloma, Taeniolella, Thalassoascus, Togninia, Teratosphaeria, Thyrospora, Thyridaria, Yarrowia, Wettsteinina, Valsaria, Ustilaginoidea, Yoshinagella, Wernerella (fungus), and/or Vismya. In a further embodiment, the set of one or more reference genomes encompasses genomes from one or more from a taxon or taxa of pathogenic microorganisms that are resistant to a particular anti-pathogen treatment. In yet a further embodiment, the anti-pathogen therapy is selected from an antibiotic treatment, antiviral treatment, antifungal treatment, or algicide. In yet a further embodiment, the MSA-aligned genomes are partitioned into overlapping 500 nt to 600 nt segments with a 200 nt to 300 nt overlap. In a certain embodiment, the candidate primer sequences are 13 bp to 15 bp in length. In another embodiment, the forward or reverse candidate primer sequences are selected from 40 nt to 60 nt regions at the ends of each segment. In yet another embodiment, a method disclosed herein further comprises the step of: (ix) chemically synthesizing a set of spiked primers that corresponds with the set of spiked primer sequences. In a further embodiment, the set of spiked primers are synthesized using an automated oligonucleotide synthesizer.

In a particular embodiment, the disclosure also provides a method of detecting a first taxon or taxa of pathogenic microorganisms in a sample, the method comprising: applying a sequencing assay to the sample to obtain sequence reads, the sequencing assay including a set of spiked primers designed by a method disclosed herein and random primers; and analyzing the sequence reads to determine whether the first taxon of pathogenic microorganisms and/or one or more other taxa of pathogenic microorganisms are present in the sample. In another embodiment, the sample is from a subject. In yet another embodiment, the subject is a human. In a further the sample is selected from whole blood, serum, plasma, urine, tissue sample, biopsy sample, isolated DNA and isolated RNA. In yet a further embodiment, the sample is a serum or urine sample. In a certain embodiment, the sample is obtained from an environmental site believed to be infected or contaminated by a taxon or taxa of pathogenic microorganisms. In another embodiment, the environmental site is a hospital room, hospice room, sewage, or contaminated water. In yet another embodiment, the sample is from a vector that is known to transmit pathogens. In a further embodiment, the vector is a mosquito, sandfly, tick, triatomine bug, tsetse fly, flea, black fly, aquatic snail, or lice. In yet a further embodiment, the sequencing assay comprises or utilizes polymerase chain reaction (PCR). In a certain embodiment, the PCR is quantitative PCR (qPCR), reverse-transcription polymerase chain reaction (RT-PCR), or reverse transcription quantitative polymerase chain reaction (RT-qPCR). In another embodiment, the sequencing assay comprises reverse transcription of a sample containing RNA using any of the primers set forth in SEQ ID NOs: 1-96, and 399-7324. In yet another embodiment, the sequencing assay provides greater than 10 sequencing reads and fewer than 100,000 sequencing reads per amplified target nucleic acid. In a further embodiment, wherein at least one, two, three, four, or more of the sequence regions targeted by the spiked primers were identified in the set of one or more reference sequences corresponding to the taxon or taxa of pathogenic microorganisms. In yet a further embodiment, the taxon or taxa of pathogenic microorganisms is present in the sample at a volume of less than 1,000 genome copies per mL. In a certain embodiment, the taxon or taxa of pathogenic microorganisms is present in the sample at a volume of less than 100 genome copies per mL. In another embodiment, the sample comprises a different taxon or taxa of pathogenic microorganisms at a volume of between 10,000-100,000 genome copies per mL. In yet another embodiment, at least one of the set of spiked primers of the sequencing assay comprises a nucleotide sequence selected from SEQ ID NOs:1-96 or 399-7324. In a further embodiment, the set of spiked primers of the sequencing assay comprises primers having nucleotide sequences of SEQ ID NOs:1-96. In yet a further embodiment, the set of spiked primers of the sequencing assay comprise primers having nucleotide sequences of SEQ ID NOs: 399-1562. In a certain embodiment, the set of spiked primers of the sequencing assay comprise primers having nucleotide sequences of SEQ ID NOs: 1563-3553. In another embodiment, the set of spiked primers of the sequencing assay comprise primers having nucleotide sequences of SEQ ID NOs: 3554-7324. In yet another embodiment, the spiked primers further comprise an adaptor sequence. In a further embodiment, the adapter sequence is positioned 5′ of the spiked primer sequences and comprises the sequence of SEQ ID NO:97. In yet a further embodiment, the random primers are random hexamers, random septamers, random octamers, and/or random nonamers. In a certain embodiment, the random primers are random hexamers and/or random nonamers. In another embodiment, the ratio of spiked primers to random primers in the sequencing assay is 2:1, 3:1, 4:1, 5:1, 6:1, 7:1, 8:1, 9:1, 10:1, or greater than 10:1. In yet another embodiment, the ratio of spiked primers to random primers in the sequencing assay is about 5:1. In a further embodiment, the sequencing assay further comprises a probe that is used to determine the amount of amplified product produced in the sequencing assay.

In a particular embodiment, the disclosure further provides for a kit comprising a set of spiked primers that comprises primers having nucleotide sequences of SEQ ID NOs:1-96, SEQ ID NOs: 399-1562, SEQ ID NOs: 1563-3553, and/or SEQ ID NOs: 3554-7324. In another embodiment, the primers further comprise an adapter sequence. In yet another embodiment, the adapter sequence is positioned 5′ of the primer sequences and comprises the sequence of SEQ ID NO:97. In a further embodiment, the kit further comprises random hexamer and/or random nonamer primers. In yet a further embodiment, the kit further comprises one or more probes having sequences selected from SEQ ID NOs:98-398.

These and other embodiments are described in more detail below.

### BRIEF DESCRIPTION OF THE DRAWINGS

FIG. 1A-C presents Zika virus sampling and sequencing in Central America and Mexico. (A) Map of Central America and Mexico. Circles indicate Zika virus sampling locations of genomes sequences generated in this study and publicly available genome sequences. (B) Temporal and geographic distribution of Zika virus RT-qPCR positive samples identified in this study. (C) Representation of the genomic sequences of 61 Zika virus genomes generated by the studies disclosed herein.

FIG. 2A-B displays the spiked primer approach for ZIKV enrichment from metagenomic libraries. (A) Flow chart of the design algorithm. Short, 13-nt primers are designed from an arbitrary set of viral reference genomes by consecutive steps of multiple sequence alignment, partitioning of the consensus sequence, and forward and reverse primer selection within 50-nt windows. (B) Diagram of exemplary metagenomic library preparation protocol. Various combinations of spiked and/or random primers, with and without adapter sequences for single-primer amplification, were tested. The protocol corresponding to conventional multiplex RT-PCR is shown for purposes of comparison. Additional bead-based enrichment using bait capture probes can be included prior to sequencing after cDNA library amplification.

FIG. 3A-B displays geographic and temporal distribution of Zika virus cases in Central America and Mexico. (A) Zika virus cases (confirmed or suspected) as measured over time plotted against climatic vector data. Each panel corresponds to a country within Central America or Mexico region. In each panel, the bar plots show available notified ZIKV case data until May 2017 (plots adapted from PAHO). In each panel, dashed lines indicate the estimated climatic vector suitability score averaged across the country. Arrows indicate the earliest confirmation of Zika virus autochthonous cases. (B) Maximum likelihood phylogeny and temporal signal of the Zika virus Asian genotype lineage. The phylogeny was estimated using PhyML based on complete and partial (>1500 nt) coding genome sequences. Statistical supports for nodes were assessed using a bootstrap approach (100 replicates). Only bootstrap supports >50 at internal nodes are displayed. Symbols at phylogenetic tips denote sampling locations of the sequences. Circles and triangles denote sequences publicly available and sequences generated in the study, respectively. The regression plot shows the correlation between the sampling date of each sequence and the genetic distance of that sequence from the root of the phylogeny. Circles denote tips of the phylogeny with its corresponding sampling location.

FIG. 4A-C demonstrates the epidemic behavior of Zika virus in Central America and Mexico. (A) Phylogeography of Zika virus in the Americas. Maximum clade credibility phylogeny estimated from complete and partial Zika virus genomes of the Asian genotype using a Bayesian molecular clock phylogeographic approach. For visual clarity, Asian and Pacific lineages are not displayed and two clades corresponding to exports to South America (34 taxa) and Caribbean (68 taxa) are collapsed and indicated respectively by the two squares. Violin shapes indicate posterior distributions of estimated dates of nodes A and B. Different gray shadings indicate the most probable ancestral lineage locations. Circles at internal nodes denote posterior probabilities >0.75. For selected nodes, numbers show the posterior probabilities of ancestral locations or clade posterior probabilities. (B) Earliest inferred dates of Zika virus spread to and within Central America and Mexico. Each box-and-whisker plot corresponds to the earliest movement between a pair of locations with well-supported virus lineage migration. Colors within box-and-whisker plots indicated pairs of countries shown in (A) and letters indicated federal states of Mexico (C: Chiapas, O: Oaxaca, G: Guerrero). The dashed line shows the estimated average climatic vector suitability score across Honduras, which is predicted to be the source of introduction and spread of Zika virus in the region of Central and Mexico. (C) Effective reproductive number through time (Rₑ) estimated using a Birth Death Skyline approach from the median posterior estimate of the time to the most recent common ancestor of the Central and Mexico clade to the most recent sample. The solid orange line, darker shading and lighter shading represent respectively the median posterior estimate, 50% and 95% highest posterior densities.

FIG. 5A-C shows genome coverage plots corresponding to ZIKV and other untargeted viruses. (A) Exemplary data obtained using methods of the present disclosure. Here, Zika virus (ZIKV) primers (referred to herein as “spiked” primers) were compared to random primers (random hexamers (N6) or random nonamers (N9)) in a 5:1 ratio, versus random primers alone. Improvements in ZIKV reads per million (RPM) and genome coverage were observed using reverse spiked primers relative to random primers alone, whereas genomic coverage of untargeted human immunodeficiency virus (HIV-1) or hepatitis C virus (HCV) present in the sample was not compromised by the use of spiked primers. (B) Exemplary data obtained using both forward and reverse spiked primers in the presence of random primers in a 5:5:1 ratio. Improvements in Zika virus RPM and genome coverage were observed using reverse and forward spiked primers relative to random primers alone. (C) exemplary data obtained using Zika virus “spiked” reverse primers and random primers in a 10:1 ratio. Increasing the ratio of spiked primers did not result in further improvements in genome coverage.

FIG. 6 shows the genome coverage of Dengue virus 1 (DENV1) obtained during metagenomic Next-Generation Sequencing of a human sample using the methods disclosed herein.

FIG. 7 shows ancestral node location posterior probabilities for node B in (FIG. 4A), estimated using the complete dataset and ten replicate subsampled datasets.

FIG. 8A-C shows exemplary mNGS sequencing data obtained using various embodiments of the present disclosure. (A) and (B) Plots of the Zika Ct value as compared to deduplicated sequencing reads that were mapped to the Zika virus genome. (C) Plot of percent genome coverage versus read coverage of deduplicated or non-deduplicated mapped Zika virus sequencing reads.

FIG. 9 provides an exemplary computer system useful for performing methods of the disclosure.

FIG. 10A-B presents metagenomic sequencing with spiked primer enrichment (MSSPE) viral primer design and metagenomic sequencing workflow. (A) Algorithm for design of viral spiked primers. A set of viral reference genomes (60 to 3,571) were aligned using MAFFT multiple sequence alignment software (Katoh and Standley, 2014), followed by partitioning of each genome into 300-500 nucleotide (nt) overlapping segments. Forward and reverse 13 nt primers (“kmers”) were selected and filtered according to specific criteria (rounded rectangular box). Using this algorithm, primers were designed for 14 RNA viruses. Spiked primer panels for arboviruses (ArboV SP; n=4), hemorrhagic fever viruses (HFV SP; n=6), and all virus (A11V SP; n=13, excluding HCV) were also constructed. (B) Metagenomic sequencing workflow. MSSPE primers are added (“spiked”) to the reaction mix during the reverse transcription step of cDNA synthesis, without adding to the overall turnaround time for the library preparation and sequencing analysis protocols. The MSSPE workflow is compatible with subsequent enrichment using tiling multiplex PCR and/or capture probes (dotted lines). Metagenomic sequence data is analyzed for pathogen identification using SURPI software.

FIG. 11A-H provides spiked primer enrichment of viral sequences using MSSPE. Shown in A-C are XY plots of the fold enrichment achieved for contrived samples containing ZIKV, DENV, EBOV, and/or MS2 bacteriophage (MS2) at defined titers and using random hexamer (RH) primers only or at spiked primer (SP) concentrations ranging from 1 M to 40 M or 80 M. (A) Enrichment of ZIKV and DENV using an arbovirus spiked primer (ArboV SP) panel, (B) Enrichment of EBOV using a hemorrhagic fever virus spiked primer (HFV SP) panel, (C) Enrichment of ZIKV, DENV, and EBOV using an all virus spiked primer (A11V SP) panel. Shown in D-G are box-and-whisker plots of the fold enrichment achieved for contrived samples containing ZIKV, DENV, and/or EBOV at titers ranging from 10 to 1,000 copies (cp)/mL. The asterisks denote virus/concentration combinations that were not tested. (D) Enrichment of ZIKV, DENV, and EBOV using virus-specific spiked primers (SP) at 4 μM concentration. (E) Enrichment of ZIKV and DENV using the ArboV SP panel at 10 μM concentration. (F) Enrichment of EBOV using the HFV SP panel at 20 μM concentration. (G) Enrichment of ZIKV, DENV, and EBOV using the A11V SP panel at 10 μM concentration. (H) Fold enrichment across all experimental replicates is plotted as a bar-and-whisker graph by detected virus or viruses and SP panel used.

FIG. 12A-E presents improvements in viral genome coverage using MSSPE. The fold coverage (y-axis) is plotted as a function of nucleotide position (x-axis). For each graph, the number of reads is normalized to the total number of viral reads obtained with no enrichment. (A) Genome coverage of the ZIKV MRC766 (Uganda) strain at 1,000 copies (cp)/mL concentration with no enrichment (top) or MSSPE enrichment using ZIKV spiked primers (ZIKV SP) (second), an arbovirus spiked primer (ArboV SP) panel (third), or an all virus spiked primer (A11V SP) panel (bottom). (B) Genome coverage of an HIV-1 Group M, CRF01 strain at 1,000 cp/mL concentration with no enrichment (left) or using HIV-1 spiked primers (HIV-1 SP) (right). (C) Genome coverage of an HCV genotype 4 strain at 10,000 cp/mL concentration with no enrichment (left) or using HCV spiked primers (HCV SP). (D) Genome coverage of a Powassan virus (POWV) strain identified in cerebrospinal fluid (CSF) from an infected patient with tickborne meningoencephalitis with no enrichment (left) or using the ArboV SP panel. (E) Genome coverage of a strain from a patient from Mexico with acute ZIKV infection during the 2013-2016 outbreak (ZIKV/Homo sapiens/MEX/2016/mex30) at ˜2,000 cp/mL concentration with no enrichment (top) or enrichment using MSSPE (second), tiling multiplex PCR (third), capture probes (fourth, using random primers alone), or MSSPE followed by capture probes (bottom). The red bars below the coverage plots show nucleotide regions with coverage of >10×, at a threshold to minimize impact from potential cross-contamination, with the overall corresponding genome coverage given in brackets.

### DETAILED DESCRIPTION

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Spring Harbor Lab Press (Cold Spring Harbor, N.Y. 1989), both of which are incorporated herein by reference. All patents, patent applications, and publications mentioned herein are incorporated herein by reference in their entireties for all purposes.

The term “a”, “an” or “the” is intended to mean “one or more”, e.g., a pathogen refers to one or more pathogenic microorganisms unless otherwise made clear from the context of the text.

The term “comprise,” and variations thereof such as “comprises” and “comprising,” when preceding the recitation of a step or an element, are intended to mean that the addition of further steps or elements is optional and not excluded.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Any methods and reagents similar or equivalent to those described herein can be used in the practice of the disclosed methods and compositions.

The disclosure provides MSSPE as a universal target enrichment method that was simple, low-cost, fast (incurring no extra turnaround time), and deployable on benchtop or portable sequencers. The MSSPE method was able to enrich reads from ZIKV, EBOV, and DENV ˜55× (median 6.3×) in low-titer clinical samples (10-10,000 copies/mL), improving detection sensitivity to 10 copies/mL and increasing genome coverage by 20-50% (average 40.3±16.3%). Notably, broad metagenomic sensitivity for pathogen detection was preserved, with enrichment of reads from emerging viruses that had not been specifically targeted a priori by the spiked primer design, including St. Louis encephalitis (SLEV), Usutu virus (USUV), and POWV, and little to no decrease in reads from off-target viruses (e.g., MS2 bacteriophage). The MSSPE method was also synergistic when combined with other enrichment strategies, further increasing the yield of viral reads for detection and gains in genome coverage. Enrichment was possible across a wide range of potential targets, from single viruses to expanded panels (arbovirus and hemorrhagic fevers) to inclusion of all spiked primers (n=4,792 to date). Taken together, the results demonstrate the potential utility of the MSSPE method for simultaneous viral diagnosis and genome surveillance of patients with unknown febrile infections in laboratory or field settings.

To enrich viruses from unknown clinical samples, the disclosure provides spiked primer panels to detect viruses associated with, e.g., arbovirus infection or hemorrhagic fever. The use of customized spiked primer panels allows potential targeting of the full range of viral pathogenic microorganisms associated with a geographic region (e.g., hemorrhagic fever viruses for testing in the DRC and arboviruses for testing in Brazil). Regional public health surveillance data can be leveraged in the design of updated spiked primer panels targeting actively circulating pathogens. This is especially important in facilitating the rapid development of diagnostic tests for a viral pathogen that is newly introduced to a geographic region (e.g., EBOV in West Africa, ZIKV in Brazil, 2009 pandemic H1N1 influenza in Mexico).

In addition to viral detection, the MSSPE method can be used for pathogen discovery and viral genome sequencing. As broad-spectrum metagenomic sensitivity for off-target pathogenic microorganisms is retained (or even occasionally enhanced, i.e. HIV with ZIKV primers), detection of novel, rare, unexpected, and/or co-infecting viruses in clinical samples is possible with MSSPE. Of note, the detection of USUV reads at lower sequencing depth required the inclusion of ArboV spiked primers, and spiked primer enrichment resulted in 18-43% increases in genome coverage for 3 emerging flaviviruses (USUV, SLEV, POWV). On average, the use of virus-specific primers improved genome recovery for 5 different viruses by 46% (±14.4%), or 36.5% (±16.8%) with the use of expanded panels (ArboV, HFV, and Ally). The minimum threshold that has been proposed for completion of a standard draft viral genome is >50%; the percentage of samples fulfilling this minimum coverage requirement increased from 15.8% (6 of 38) using mNGS alone to 81.6% (31 of 38) using MSSPE primers (Chi-squared test, p<0.0001).

The MSSPE method described herein is also effective in genome sequencing of recombinant HIV viruses, both circulating and unknown recombinant forms, which can also exhibit sequence divergence of up to 35% in the env gene, as well as multiple HCV genotypes. Interestingly, enrichment using MSSPE was occasionally noted for unrelated, off-target viruses, as in the case of HIV enrichment using ZIKV primers, or USUV enrichment using HIV primers. In the former case, enrichment was secondary to less binding of RH primers to background targets, including human host and contaminant bacterial and plant sequences. In the latter case, enrichment was likely secondary to 13 nt HIV-1 spiked primers exhibiting high homology to the USUV genome sequence. These results provide a proof-of-concept that clinical samples originally earmarked for viral genomic surveillance can be simultaneously screened using MSSPE for viral pathogen discovery efforts.

In addition to robust target enrichment, advantages of the MSSPE method include (i) a simple and convenient protocol, which does not incur extra time nor require additional reagents beyond the spiked primer mixes, (ii) compatibility with multiple library preparation protocols (e.g., transposon or adapter ligation-based, since the target enrichment occurs during the reverse transcription step), (iii) lack of apparent cross-contamination, (iv) low cost, and (v) synergy with other enrichment methods. With extremely low-titer samples (<10 copies/mL or Ct>40), especially on low-throughput, error prone platforms such as the MinION nanopore sequencer, combining MSSPE with a complementary tiled multiplex PCR or capture probe enrichment approach may be useful, as is shown herein and done previously for studying the genomic epidemiology of ZIKV in CAM. The MSSPE approach can also be extended for enrichment of DNA targets, such as identification of nonviral (bacterial, fungal, or parasitic) pathogenic microorganisms and antimicrobial resistance genes.

As used herein, the term “pathogen” refers to a virus, bacterium, protozoa, prion, archaea, fungus, algae, parasite, or other microbe (helminth) that causes or induces disease or illness in a subject. The term includes both the disease-causing organism per se (e.g., its genome) and toxins produced by the pathogen (e.g., Shiga toxins) present in a sample of the subject. Detection of a pathogen as set forth in the methods disclosed herein includes detection of a portion of the genome of the pathogen or a nucleic acid molecule that is complementary or substantially complementary (i.e., at least 90% complementary) to a portion of the genome of the pathogen.

With respect to the term “particular taxon of pathogens”, the term refers to classification or taxonomy of pathogens. Accordingly, a “particular taxon of pathogens” can include pathogenic microorganisms classified at various levels of taxonomic rank, e.g., by Realm (Riboviria), Domain/SubRealm (e.g., Bacteria, Arachaea), by Kingdom (e.g., Protista, Fungi, etc.), by Phylum (e.g., Vira, Chlamydiae, etc.), by Class (e.g., Chlamydiales, Parachlamydiales, etc.), by Order (e.g., caudovirales, herpesvirales, ligamenvirales, mononegavirales, etc.), by Family (e.g., Reoviridae, Caliciviridae, Flaviviridae, Orthomyxoviridae, Picornaviridae, Togaviridae, Paramyxoviridae, Bunyaviridae, Rhabdoviridae, Filoviridae, Coronaviridae, Astroviridae, Bornaviridae, Arteriviridae, Hepeviridae, Retroviridae, etc.), or by Genus (e.g., Hepacivirus, flavivirus, pegivirus, pestivirus, etc.). Thus, “a particular taxon of pathogens” refers to a group of related species that share significant properties, but may differ in host range and virulence. An exemplary taxonomic classification system of viruses suitable for use with the disclosure is the international committee on Taxonomy of viruses (ICTV) which organizes viruses based on the structure and composition of viruses. For example, the ICTV database, freely available at [https://]talk.ictvonline.org/ictv-reports/ictv_online_report/ (note the “https” has been bracketed to remove active hyperlinks) classifies viruses as either ssDNA viruses, ssDNA/dsRNA viruses, dsDNA viruses, dsRNA viruses, reverse transcribing DNA and RNA viruses, negative sense RNA viruses and positive sense RNA viruses. For purposes of this disclosure, “a particular taxon of viruses” will refer to a Family or Genus taxonomic level of related viruses. Typically, viral genus names end in the suffix -virus. Viral genera contemplated for use with the disclosure include any of the viral genera or viral species provided in List 1 and List 2, respectively.

List 1: Viral Genera:

Ahjdlikevirus, Alfamovirus, Allexivirus, Allolevivirus, Alphabaculovirus, Alphacarmotetravirus, Alphacoronavirus, Alphaentomopoxvirus, Alphafusellovirus, Alphaguttavirus, Alphalipothrixvirus, Alphamesonivirus, Alphanecrovirus, Alphanodavirus, Alphanudivirus, Alphapapillomavirud, Alphapartitivirus, Alphapermutotetravirus, Alpharetrovirus, Alphasphaerolipovirus, Alphaspiravirus, Alphatorquevirus, Alphaturrivirus, Alphavirus, Amalgavirus, Ambidensovirus Amdoparvovirus, Ampelovirus, Ampullavirus, Andromedalikevirus, Anulavirus, Aparavirus, Aphthovirus, Apscaviroid, Aquabirnavirus, Aquamavirus, Aquaparamyxovirus, Aquareovirus, Arterivirus, Ascovirus, Asfivirus, Atadenovirus, Aureusvirus, Aurivirus Avastrovirus, Avenavirus, Aveparvovirus, Aviadenovirus, Avibirnavirus, Avihepadnavirus, Avihepatovirus, Avipoxvirus, Avisivirus, Avsunviroid, Avulavirus, Bacillarnavirus, Babuvirus, Bacilladnavirus, Barnavirus, Badnavirus, Bafinivirus, Bcep22likevirus, Barnyardlikevirus, Batrachovirus, Bdellomicrovirus, Bcep78likevirus, Bcepmulikevirus, Benyvirus, Becurtovirus, Begomovirus, Betaentomopoxvirus, Betabaculovirus, Betacoronavirus, Betalipothrixvirus, Betafusellovirus, Betaguttavirus, Betanudivirus, Betanecrovirus, Betanodavirus, Betaretrovirus, Betapapillomavirus, Betapartitivirus, Betatorquevirus, Betasphaerolipovirus, Betatetravirus, Bignuzlikevirus, Bicaudavirus, Bidensovirus, Bornavirus, Blosnavirus, Bocaparvovirus, Bracovirus, Botrexvirus, Bppunalikevirus, Bromovirus, Brambyvirus, Brevidensovirus, Bronlikevirus, Cafeteriavirus, Bymovirus, Cardiovirus, C2likevirus, C5likevirus, Carmovirus, Capillovirus, Capripoxvirus, Cervidpoxvirus, Cardoreovirus, Carlavirus, Che9clikevirus, Caulimovirus, Cavemovirus, Chipapillomavirus, Charlielikevirus, Che8likevirus, Chlorovirus, Cheravirus, Chilikevirus, Circovirus, Chlamydiamicrovirus, Chloriridovirus, Clavavirus, Chrysovirus, Cilevirus, Coccolithovirus, Citrivirus, Cjwunalikevirus, Comovirus, Closterovirus, Cocadviroid, Corticovirus, Coleviroid, Coltivirus, Cp8unalikevirus, Copiparvovirus, Corndoglikevirus, Crocodylidpoxvirus, Cosavirus, Cp220likevirus, Cuevavirus, Crinivirus, Cripavirus, Cyprinivirus, Cryspovirus, Cucumovirus, Cytorhabdovirus, Curtovirus, Cypovirus, Cystovirus, Cytomegalovirus, D3likevirus, Deltalipothrixvirus, Deltabaculovirus, D3112likevirus, Deltaretrovirus, Deltapapillomavirus, Deltacoronavirus, Dependoparvovirus, Deltatorquevirus, Deltapartitivirus, Dinodnavirus, Dianthovirus, Deltavirus, Dyodeltapapillomavirus, Dinornavirus, Dicipivirus, Dyoiotapapillomavirus, Dyoepsilonpapillomavirus, Dinovernavirus, Dyomupapillomavirus, Dyokappapapillomavirus, Dyoetapapillomavirus, Dyopipapillomavirus, Dyonupapillomavirus, Dyolambdapapillomavirus, Dyothetapapillomavirus Dyorhopapillomavirus, Dyoomikronpapillomavirus, Dyopipapillomavirus, Dyosigmapapillomavirus, Dyozetapapillomavirus, Emaravirus, Enterovirus, Ebolavirus, Epsilon15likevirus, Enamovirus, Elaviroid, Epsilontorquevirus, Entomobirnavirus, Endornavirus, Errantivirus, Epsilonpapillomavirus, Ephemerovirus, Etatorquevirus, Eragrovirus, Epsilonretrovirus, Fabavirus, Erythroparvovirus, Erbovirus, Fijivirus, Felixounalikevirus, Etapapillomavirus, Furovirus, Flavivirus, F116likevirus, Ferlavirus, Foveavirus, Gallivirus, Gammabaculovirus, Gammaentomopoxvirus, Gammalipothrixvirus, Gammapartitivirus, Gammaretrovirus, Gallantivirus, Gammatorquevirus, Giardiavirus, Gammacoronavirus, Glossinavirus, Gyrovirus, Gammapapillomavirus, Gammasphaerolipovirus, Globulovirus, Halolikevirus, Hantavirus, Hemivirus, Hempavirus, Hepandensovirus, Hepatovirus, Hapunalikevirus, Hk578likevirus, Hordeivirus, Hepacivirus, Hpunalikevirus, Hunnivirus, Higrevirus, Hostuviroid, Hypovirus, Ichtadenovirus, I3likevirus, Idnoreovirus, Ictalurivirus, Ilarvirus, Iebhlikevirus, Ichnovirus, Influenzavirus B, Iltovirus, Idaeovirus, Iotapapillomavirus, Influenzavirus C, Iflavirus, Iridovirus, Iotatorquevirus, Influenzavirus A, Inovirus, Ipomovirus, Isavirus, Iteradensovirus, Jerseylikevirus, Kappapapillomavirus, Kappatorquevirus, Kobuvirus, Kunsagivirus, Labyrnavirus, Lambdapapillomavirus, Lagovirus, Lentivirus, Lambdatorquevirus, L5likevirus, Lolavirus, Leporipoxvirus, Lambdalikevirus, Lymphocryptovirus, Luteovirus, Leishmaniavirus, Lymphocystivirus, Levivirus, Luz24likevirus, Lyssavirus, Machlomovirus, Mamastrovirus, Macanavirus, Marafivirus, Macluravirus, Marnavirus, Mammarenavirus, Macavirus, Mastrevirus, Marburgvirus, Maculavirus, Megrivirus, Marseillevirus, Mandarivirus, Microvirus, Megabirnavirus, Mardivirus, Mischivirus, Metapneumovirus, Mastadenovirus, Morbillivirus, Mimivirus, Megalocytivirus, Mupapillomavirus, Mitovirus, Metavirus, Mycollexivirus, Mosavirus, Mimoreovirus, Muromegalovirus, Molluscipoxvirus, Mycoreovirus, Mulikevirus, Muscavirus, N15likevirus, Nanovirus, Nepovirus, N4likevirus, Nucleorhabdovirus, Narnavirus, Nairovirus, Norovirus, Nebovirus, Nupapillomavirus, Novirhabdovirus, Nyavirus, Omegalikevirus, Omikronpapillomavirus, Orthobunyavirus, Okavirus, Orthopoxvirus, Omegapapillomavirus, Oleavirus, Oscivirus, Ophiovirus, Omegatetravirus, Orthohepadnavirus, Orbivirus, Orthoreovirus, Orthohepevirus, Ostreavirus, Oryzavirus, Ourmiavirus, Parechovirus, Pbiunalikevirus, Pegivirus, P2likevirus, P22likevirus, Percavirus, Panicovirus, P23likevirus, Petuvirus, Pasivirus, Parapoxvirus, Phi29likevirus, Pbunalikevirus, Passerivirus, Phicd119likevirus, Pelamoviroid, Pecluvirus, Phietalikevirus, Perhabdovirus, Penstyldensovirus, Phijlunalikevirus, Pgonelikevirus, Pestivirus, Phipapillomavirus, Phic3unalikevirus, Phaeovirus, Picobirnavirus, Phie125likevirus, Phicbklikevirus, Plasmavirus, Phyllikevirus, Phieco32likevirus, Poacevirus, Phikmvlikevirus, Phihlikevirus, Polyomavirus, Phlebovirus, Phikzlikevirus, Potexvirus, Pipapillomavirus, Phytoreovirus, Proboscivirus, Plectrovirus, Piscihepevirus, Pseudovirus, Polemovirus, Pneumovirus, Punalikevirus, Pomovirus, Polerovirus, Potyvirus, Pospiviroid, Protoparvovirus, Prasinovirus, Prymnesiovirus, Psimunalikevirus, Psipapillomavirus, Quaranjavirus, Quadrivirus, Raphidovirus, Reylikevirus, Rhopapillomavirus, Reptarenavirus, Roseolovirus, Rhadinovirus, Rubulavirus, Rosadnavirus, Ranavirus, Rotavirus, Respirovirus, Rubivirus, Rudivirus, Rymovirus, Rhizidiovirus, Rosavirus, Salivirus, Sap6likevirus, Schizot4virus, Sadwavirus, Seadornavirus, Salmonivirus, Sequivirus, Sapelovirus, Siadenovirus, Sclerodarnavirus, Sakobuvirus, Sigmavirus, Semotivirus, Salterprovirus, Skunalikevirus, Sfi1unalikevirus, Sapovirus, Soymovirus, Sicinivirus, Scutavirus, Spiromicrovirus, Simplexvirus, Senecavirus, Spumavirus, Sobemovirus, Sfi21dtunalikevirus, Sp6likevirus, Sigmapapillomavirus, Spounalikevirus, Sirevirus, Suipoxvirus, Solendovirus, Spbetalikevirus, Sprivivirus, T4virus, Taupapillomavirus, Tepovirus, Thetapapillomavirus, T5likevirus, Tibrovirus, Tectivirus, Tobravirus, Teschovirus, T7likevirus, Torovirus, Thetatorquevirus, Tenuivirus, Totivirus, Tm4likevirus, Tetraparvovirus, Trichomonasvirus, Tombusvirus, Thogotovirus, Tunalikevirus, Torradovirus, Tobamovirus, Turncurtovirus, Tp2unalikevirus, Topocuvirus, Trichovirus, Tospovirus, Tungrovirus, Tremovirus, Twortlikevirus, Tritimovirus, Tupavirus, Tymovirus, Umbravirus, Upsilonpapillomavirus, Varicellovirus, Velarivirus, Vesiculovirus, Victorivirus, Vitivirus, Varicosavirus, Vesivirus, Viunalikevirus, Waikavirus, Wbetalikevirus, Whispovirus, Xp10likevirus, Xipapillomavirus, Yualikevirus, Yatapoxvirus, Zetapapillomavirus, Zetatorquevirus, and Zeavirus.

List 2: Viral Species:

West Nile Virus, dengue virus, tick-borne encephalitis virus, Japanese encephalitis virus, yellow fever virus, Zika virus, cell fusing agent virus, Palm Creek virus and/or Parramatta River virus. However, it should be understood that any other classification or taxonomic ranking of viruses is contemplated by the present disclosure.

Bacteria and fungi are also routinely classified or ranked based on different taxa corresponding to genus, family, and species identification. For example, fungal taxon contemplated by the disclosure include any of the fungal taxon provided in List 3 or List 4. It will be apparent to one of ordinary skill in the art that List 3 and List 4 are not exhaustive and is provided as an exemplary list.

List 3: Fungal Genera:

Anaeromyces, Caecomyces, Allomyces, Entyloma, Diskagma, Blastocladia, Funneliformis, Entylomella, Coelomomyces, Glomus (fungus), Fusidium, Heptameria, Holmiella, Homostegia, Hyalocrea, Hyalosphaera, Hypholoma, Hypobryon, Hysteropsis, Koordersiella, Karschia, Kirschsteiniothelia, Lembosiopeltis, Kullhemia, Kusanobotrys, Leptodothiorella, Lanatosphaera, Lasiodiplodia, Leveillina, Lepidopterella, Lepidostroma, Lollipopaia, Leptosphaerulina, Leptospora, Macrovalsaria, Lichenostigma, Licopolia, Massariola, Lopholeptosphaeria, Maireella, Microdothella, Macroventuria, Microcyclella, Mycoglaena, Melanodothis, Montagnella, Mycoporopsis, Moniliella, Mycopepon, Myriangium, Mycomicrothelia, Mycothyridium, Mytilostoma, Mycosphaerella, Mytilinidion, Neofusicoccum, Myriostigmella, Neocallimastix, Oomyces, Neopeckia, Orpinomyces, Ostreichnion, Ophiosphaerella, Paropodia, Passeriniella, Passerinula, Pedumispora, Peyronellaea, Phaeoacremonium, Phaeocyrtidula, Phaeoglaena, Phaeopeltosphaeria, Phaeoramularia, Phaeosperma, Phaneromyces, Phialophora, Philonectria, Phragmocapnias, Phragmosperma, Piedraia, Piromyces, Placocrea, Placostromella, Plagiostromella, Plejobolus, Pleostigma, Polychaeton, Pseudocercospora, Pseudocryptosporella, Pseudogymnoascus, Pseudothis, Pycnocarpon, Rhytidhysteron, Rhizophagus (fungus), Rhopographus, Rosellinula, Rhytisma, Robillardiella, Roussoëllopsis, Rosenscheldia, Rostafinskia, Sarcopodium, Savulescua, Saksenaeaceae, Scolecobonaria, Scolicotrichum, Schizoparme, Semifissispora, Septoria, Scorias, Sphaceloma, Sphaerellothecium, Spathularia, Stagonosporopsis, Stenella (fungus), Sphaerulina, Stigmina (fungus), Stioclettia, Stigmidium, Sydowia, Tephromela, Stuartella, Teichosporella, Thalloloma, Taeniolella, Thalassoascus, Togninia, Teratosphaeria, Thyrospora, Thyridaria, Yarrowia, Wettsteinina, Valsaria, Ustilaginoidea, Yoshinagella, Wernerella (fungus), and Vismya.

List 4: Fungi Species:

Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

Additionally, bacterial taxon contemplated by the disclosure include any of the bacterial taxon provided in List 5 or List 6. It will be apparent to one of ordinary skill in the art that List 5 and List 6 are not exhaustive and is provided as an exemplary list.

List 5: Bacterial Genera:

Heliobacter, Aerobacter, Rhizobium, Agrobacterium, Bacillus, Clostridium, Pseudomonas, Xanthomonas, Nitrobacteriaceae, Nitrobacter, Nitrosomonas, Thiobacillus, Spirillum, Vibrio, Bacteroides, Corynebacterium, Listeria, Escherichia, Klebsiella, Salmonella, Serratia, Shigella, Erwinia, Rickettsia, Chlamydia, Mycoplasma, Actinomyces, Streptomyces, Mycobacterium, Polyangium, Micrococcus, Staphylococcus, Lactobacillus, Diplococcus, Streptococcus, and Campylobacter.

List 6: Bacterial Species:

Actinomyces israelii, Bacillus anthraces, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

With respect to the term “different taxon of pathogens”, the term is distinct from the “particular taxon of pathogens”. Here, the different taxon of pathogenic microorganisms does not overlap with the particular taxon of pathogens. For example, if a particular taxon of pathogenic microorganisms includes the family of Flavivirus, the different taxon of pathogenic microorganisms does not include Flavivirus but can include another family of viruses, such as Alphaviruses, bacterial, fungal, archaea, algal, protozoan, and/or parasitic pathogens. If the particular taxon of pathogenic microorganisms and different taxon of pathogenic microorganisms are from the same domain (e.g., bacterial domain), the two taxa identified by the method are distinct.

As used herein, the term “sample” refers to a sample collected from a subject including, but not limited to, human and non-human animal subjects, that may be affected by or are suspected of infection by a pathogen (e.g., an infectious virus, bacterium, protozoa, prion, fungi, algae, parasite or other microbe). The term also includes samples collected from the environment including, but not limited to, surface samples, water samples, soil samples and the like. A sample includes but is not limited to, a cell, cell lysate, isolated DNA, isolated RNA, tissue section, tissue biopsy, liquid biopsy, blood, or other biological fluid (e.g., cerebrospinal fluid) obtained from a subject. A sample includes blood samples (e.g., whole peripheral blood, serum or plasma), tissue samples (e.g., fresh, frozen or Fixed Formalin Paraffin Embedded (FFPE) samples, biopsy samples (e.g., fine needle aspirates (FNAs)), excretions and secretions such as, saliva, sputum, urine, stool, plasma/serum, breast milk, sperm, semen, vaginal secretions, sweat, mucus, bile, and oral and genital mucosal swabs. The sample can include a clinical sample (e.g., a patient sample) for the purpose of diagnosis, detection, epidemiology, treatment, disease monitoring, and the like. In some instances, the sample comprises isolated RNA and/or DNA from a mammal (e.g., pig, cow, goat, sheep, rodent, rat, mouse, dog, cat, non-human primate or human). A tissue sample typically includes one or more cells obtained from a tissue of the subject or cells derived from a tissue obtained from the subject (e.g., cells in tissue culture). It will be apparent to one of ordinary skill in the art that a tissue sample can include cells obtained from a somatic tissue (e.g., liver, kidney, spleen, gall bladder, stomach, bladder, uterus, intestines, pancreas, colon, lung, heart, brain, muscle, bone, pharynx and larynx).

As used herein, the term “subject” refers to any member of the class animals, including, without limitation, humans and other primates, including non-human primates such as rhesus macaques, chimpanzees and other monkey and ape species; farm animals, such as cattle, sheep, pigs, goats and horses; domestic mammals, such as dogs and cats; laboratory animals, including rabbits, mice, rats and guinea pigs; birds and other reptiles, including domestic, wild, and game birds, such as chickens, turkeys, geese, ducks, lizards, alligators, and snakes; amphibians, including frogs, toads, salamanders, and newts; fish, such as salmon, and tilapia; and insects. The term does not denote a particular age or gender. Thus, adult, young, and newborn subjects are intended to be included as well as male and female subjects. In most instances, the subject is a host to the pathogen and the pathogen may rely on its ability to infect the host, for example the production of toxins, to enter cells and tissues within the host, and acquire host nutrients to maintain infectiousness. The term includes subjects who are experiencing or have experienced illness or disease associated with a particular taxon of pathogenic microorganisms or subjects who are infected (or suspected of being infected) with a particular taxon of pathogen but are not experiencing or demonstrating symptoms of illness or disease associated with the pathogen.

As used herein, a “target” refers to a molecule of interest to be detected in a sample. In some embodiments, the target is a nucleic acid molecule. In a one embodiment, the target is a target DNA, target RNA or target nucleic acid from a pathogen. In some embodiments, the target is a polynucleotide, such as dsDNA or ssDNA; RNA, such as ssRNA or dsRNA, or a DNA-RNA hybrid. In some embodiments, two or more target molecules are detected in a single sample. In some embodiments, the two or more target molecules may be related to each other (e.g., nucleic acids from the same taxon, genus or species of pathogens). In another embodiment, a first target molecule is from a first taxon of pathogenic microorganisms and a second target molecule is from a second taxon of pathogens. In some embodiments, the target nucleic can be from the host subject and not a pathogen.

In some instances, a target sequence or target nucleic acid molecule refers to a region, subsequence, or complete nucleic acid molecule which is to be amplified (e.g., RNA to cDNA, or amplification of DNA) or detected using the method, kits and compositions disclosed herein. Accordingly, amplification of one or more target sequences can include detection of one or more pathogenic microorganisms in a single sample from a subject, such as but not limited to, the detection and/or identification of a co-infection in the sample from the subject. For example, a clinical sample from a subject (e.g., a serum or urine sample from a human subject) can be evaluated for the presence (or absence) of an amplified target sequence present in the genome of a virus or bacterium. Identification of two target sequences from distinct taxa from different domains (e.g., bacterial and viral domains) would be indicative that the subject is infected by both pathogenic microorganisms (e.g., a viral pathogen and a bacterial pathogen). Identification of the target sequence in the sample can be useful for the modulation of the form, dosage, or regime of treatment for the subject affected by the pathogen.

As used herein, the terms “treatment” and “treating” and the like, refer to methods or compositions for amelioration of disease or illness including any objective or subjective parameter such as abatement; remission; diminishing of symptoms or delaying the onset of symptoms; slowing in the rate of degeneration or decline; making the final point of degeneration less debilitating; and/or improving a subject's physical or mental well-being.

As used herein, the term “amplifying” refers to the process of synthesizing nucleic acid molecules that are complementary to one (or both strands) of a template nucleic acid molecule (e.g., nucleic acid molecules from the Zika virus genome). Amplifying a nucleic acid molecule typically includes denaturing the template nucleic acid, particularly if the template nucleic acid is double-stranded, annealing primers to the template nucleic acid at a temperature that is below the melting temperatures of the primers, and enzymatically elongating from the primers to generate an amplification product. Generally, synthesis initiates at the 3′ end of a primer and proceeds in a 5′ to 3′ direction along the template nucleic acid strand. Amplification typically requires the presence of deoxyribonucleoside triphosphates, a polymerase enzyme (e.g., DNA or RNA polymerase or T7 for in vitro transcription in TMA) and an appropriate buffer and/or co-factors for optimal activity of the polymerase enzyme (e.g., MgCl₂ and/or KCl).

As used herein, the term “primer” refers to oligomeric compounds, primarily to oligonucleotides containing naturally occurring nucleotides such as adenine, guanine, cytosine, thymine and/or uracil, but may also include modified oligonucleotides (e.g., modified nucleotides, nucleosides, synthetic nucleotides having modified base moieties and/or modified sugar moieties (See, Protocols for Oligonucleotide Conjugates, Methods in Molecular Biology, Vol 26, (Sudhir Agrawal, Ed., Humana Press, Totowa, N.J., (1994)); and Oligonucleotides and Analogues, A Practical Approach (Fritz Eckstein, Ed., IRL Press, Oxford University Press, Oxford) that are able to prime DNA synthesis by an enzyme, typically in a template-dependent manner, i.e., the 3′ end of the primer provides a free 3′-OH group to which further nucleotides are attached by the enzyme (e.g., DNA polymerase or reverse transcriptase) establishing a 3′ to 5′ phosphodiester linkage whereby deoxynucleoside triphosphates are used and pyrophosphate is released. Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066. A review of synthesis methods is provided in Goodchild, 1990, Bioconjugate Chemistry 1(3):165-187.

A primer is typically a single-stranded deoxyribonucleic acid. The appropriate length of a primer depends on the intended use of the primer but typically ranges from 6 to 50 nucleotides. Short primer molecules (e.g., having a length within a range of 11-17 nucleotides) generally require cooler temperatures to form sufficiently stable hybrid complexes with a template nucleic acid. The design of suitable primers for the amplification of a given target nucleic acid sequence is well known in the art and publicly available software such as, but not limited to, Primer3, NetPrimer, can be used to input a target sequence of interest to obtain optimized primer(s) to reduce off-target or secondary structure considerations.

As used herein, “hybridization”, “hybridizing”, “anneal” and “annealing”, and the like, refer to a process of combining two complementary (or substantially complementary (i.e., at least 90%) single-stranded DNA or RNA molecules so as to form a single, double-stranded molecule (DNA/DNA, DNA/RNA, RNA/RNA) through conventional hydrogen base pairing. Hybridization stringency is typically determined by the hybridization temperature and salt concentration of the hybridization buffer; e.g., high temperature and low salt provide high stringency hybridization conditions. Examples of salt concentration ranges and temperature ranges for different hybridization conditions are as follows: high stringency, approximately 0.01 M to approximately 0.05 M salt, hybridization temperature 5° C. to 10° C. below Tm; moderate stringency, approximately 0.16 M to approximately 0.33 M salt, hybridization temperature 20° C. to 29° C. below Tm; and low stringency, approximately 0.33 M to approximately 0.82 M salt, hybridization temperature 40° C. to 48° C. below Tm of duplex nucleic acids is calculated by standard methods well-known in the art (see, e.g., Maniatis, T., et al., Molecular Cloning: A Laboratory Manual, Cold Spring Harbor Laboratory Press: New York (1982); Casey, J., et al., Nucleic Acids Research 4:1539-1552 (1977); Bodkin, D. K., et al., Journal of Virological Methods 10(1):45-52 (1985); Wallace, R. B., et al., Nucleic Acids Research 9(4):879-894 (1981)). Algorithm prediction tools to estimate Tm are also publicly available (see, e.g., [http://] [tmcalculator.neb.com]). High stringency conditions for hybridization typically refer to conditions under which a nucleic acid molecule having complementarity (or substantial complementarity, e.g., greater than 90%, 95%, 98%, 99% complementarity) to a target sequence predominantly hybridizes with the target sequence and does not hybridize to non-target or off-target sequences.

In some embodiments, hybridizing refers to the annealing of a primer to a complementary (or substantially complementary (i.e., greater than 90% complementary)) RNA or DNA sequence obtained from a pathogen. In another embodiment, hybridizing can include annealing at least one probe to an amplification product (e.g., cDNA molecule) derived from a pathogen. Hybridization conditions typically include a temperature below the melting temperature of the primers or probes to reduced non-specific hybridization of the primers/probes. Accordingly, in some embodiments of the disclosure, hybridization conditions are of moderate stringency or high stringency.

As used herein, the term “thermostable polymerase” refers to a polymerase enzyme that is heat stable, i.e., the enzyme catalyzes the formation of a primer extension product complementary to a template nucleic acid, and is not irreversibly denatured when subjected to elevated temperatures for the time needed to effect denaturation of double-stranded template nucleic acids (e.g., between 95° C.-99° C.). Thermostable polymerases have been isolated from Thermus flavus, T. ruber, T. thermophilus, T. aquaticus, T. lacteus, T. rubens, Bacillus stearothermophilus, and Methanothermus fervidus. Additionally, polymerases that are not thermostable can be employed in the PCR assays disclosed herein, for example by replenishing the polymerase between synthesis/extension and denaturation steps as it becomes denatured. Any polymerase or thermostable polymerase known in the art is suitable for use in the method disclosed herein.

As used herein, the term “complement thereof” or “complementary” refers to a nucleic acid molecule that is optionally the same length as a target molecule of interest and possesses a structural (e.g., nucleotide) composition that is complementary (i.e., capable of conventional hydrogen base pairing) with the target molecule of interest, unless otherwise specified. Substantial complementarity refers to a nucleic acid molecule that is optionally the same length as the target molecule of interest but is greater than 90% complementary and less than 100% complementary to the target molecule of interest.

As used herein, the terms “extension”, “extend” or “elongation” when used with respect to nucleic acid molecules refers to a biological process by which additional nucleotides (or nucleotide analogs) are incorporated into nucleic acid molecules. For example, a nucleic acid can be extended by a nucleotide incorporating enzyme, such as a polymerase or reverse transcriptase that typically adds sequentially, a nucleotide to the 3′ terminal end of the nucleic acid molecule (e.g., the freely available 3′-OH group).

As used herein, the terms “identical” or “percent identity” in the context of two or more nucleic acid sequences, refers to two or more sequences that are the same or have a specified percentage of nucleotides that are the same (i.e., identical), when compared and aligned for maximum correspondence, e.g., as measured using one of the sequence comparison algorithms or by visual inspection. An exemplary algorithm that is suitable for determining percent sequence identity and sequence similarity is the BLAST program, which are described in Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:113-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656.

Other exemplary multiple sequence alignment computer programs include MAFFT ([https://] [mafft.cbrc.jp/alignment/software/]), MUSCLE ([https://] [www.ebi.ac.uk/Tools/msa/muscle/]), and CLUSTALW ([https://] [www.ebi.ac.uk/Tools/msa/clustalw2/]). Percent identity between two nucleic acid sequences is generally calculated using standard default parameters of the various methods or computer programs. A high degree of sequence identity, as used herein, between two nucleic acid molecules is typically at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, or any range of percent identity that includes or is between any two of the foregoing percentages (e.g., between 90% identity and 100% identity, between 95% identity and 98% identity, etc.). A moderate degree of sequence identity, as used herein, between two nucleic acid molecules is typically at least 80% identity, at least 82% identity, at least 83% identity, at least 84% identity, at least 85% identity, at least 86% identity, at least 87% identity, at least 88% identity, at least 89% identity, or any range of percent identity that includes or is between any two of the foregoing percentages (e.g., between 80% identity and 90% identity, between 85% identity and 89% identity, etc.). A low degree of sequence identity, as used herein, between two nucleic acid molecules is typically at least 50% identity, at least 55% identity, at least 60% identity, at least 65% identity, at least 70% identity, at least 75% identity, at least 79% identity, or any range of percent identity that includes or is between any two of the foregoing percentages (e.g., between 50% identity and 70% identity, 55% identity and 75% identity). For example, a sample from a subject, (e.g., suspected of being infected with Zika virus) can have a high degree of sequence identity to a reference taxon of pathogenic microorganisms (e.g., Flavivirus) and a low degree of sequence identity to bacterial pathogenic microorganisms (e.g., Streptococcus, Clostridium, Salmonella and Mycobacterium).

As used herein, the terms “nucleic acid”, “polynucleotide” and “oligonucleotide” refer to a polymeric form of nucleotides. The nucleotides may be deoxyribonucleotides (DNA), ribonucleotides (RNA), analogs thereof, or combinations thereof, and may be of any length. Polynucleotides may perform any function and may have any secondary and tertiary structures (e.g., hairpins, stem loop structures). Oligonucleotides refer to polymeric form of nucleotides typically having much shorter lengths than polynucleotides (e.g., <50 nt). The terms encompass known analogs of natural nucleotides and nucleotides that are modified in the base, sugar and/or phosphate moieties. Preferably, analogs of a particular nucleotide have the same base-pairing specificity (e.g., an analog of A base pairs with T). An oligonucleotide may comprise one modified nucleotide or multiple modified nucleotides. Examples of modified nucleotides include fluorinated nucleotides, methylated nucleotides, and nucleotide analogs. The nucleotide structure may be modified before or after a polymer is assembled. The terms also encompass nucleic acids comprising modified backbone residues or linkages that are synthetic, naturally occurring, and non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA) and morpholino structures.

As used herein, a “modified nucleotide” or “nucleotide analog” in the context of an oligonucleotide, primer or probe, refers to incorporation of a non-naturally occurring nucleotide (e.g., a nucleotide other than A, G, T, C or U) within the oligonucleotide, primer or probe, and whereby incorporation of the modified nucleotide or nucleotide analog does not hinder or prevent nucleic acid extension or elongation under suitable amplification conditions. Examples of nucleic acid modifications are described in, e.g., U.S. Pat. No. 6,001,611. Other modified nucleotide substitutions may alter the stability of the oligonucleotide (e.g., modulate its Tm), or provide other desirable features (e.g., nuclease resistance).

As used herein, a “reagent” refers broadly to any agent used in a reaction, other than the analyte (e.g., nucleic acid molecule being analyzed). Illustrative reagents for a nucleic acid amplification reaction or sequencing assay include, but are not limited to, buffer, metal ions, polymerase, reverse transcriptase, primers, probes, template nucleic acid, nucleotides, labels, dyes, nucleases, adapters, oligo-coated beads, microparticles or droplets, and the like. Generally, reagents for enzymatic reactions include, for example, substrates, cofactors, buffers, metal ions, inhibitors, and/or activators.

The disclosure also provides embodiments directed to dehosting a sample prior to the identification of a taxon or taxa of pathogenic microorganisms in a sample. Such dehosting techniques and compositions relate to the selective cleavage of non-microbial nucleic acids in a sample containing both pathogen-based nucleic acids and non-pathogen-based nucleic acids (e.g., nucleic acids from a subject), so that the sample becomes greatly enriched with microbial nucleic acids. Examples of dehosting methods include those described in Feehery et al., PLoS ONE 8:e76096 (2013); Sachse et al., Journal of Clinical Microbiology 47:1050-1057 (2009); Barnes et al., PLoS ONE 9(10):e109061 (2014); Leichty et al., Genetics 198(2):473-81 (2014)); Hasan et al., J Clin Microbiol 54(4):919-27 (2016); and Liu et al., PLoS ONE 11(1):e0146064 (2016). Additionally, commercial kits for carrying out dehosting are also available, including the NEBNext Microbiome DNA Enrichment™ Kit, the Molzym MolYsis Basic™ kit, and MICROBEEnrich™ Kit.

In some embodiments, the dehosting methods and compositions disclosed herein takes advantage of properties associated with non-pathogen-based nucleic acids, including methylation at CpG residues, and associations with DNA-binding proteins, such as histones. For example, in a particular embodiment the dehosting methods and compositions can utilizes a nucleic acid binding protein that selectively binds with non-pathogen-based nucleic acids (e.g., histones, restriction enzymes). In a further embodiment, the dehosting methods and compositions can comprise a recombinant protein that selectively binds with non-pathogen-based nucleic acids, and which also selectively degrades non-pathogen-based nucleic acids, i.e., the recombinant protein comprises both a nonmicrobial nucleic acid binding domain and a nuclease domain. In a particular embodiment, the nucleic acid binding protein is a histone. Histones are found in the nuclei of eukaryotic cells, and in certain Archaea, namely Thermoproteales and Euryarchaea, but not in bacteria or viruses. In a further embodiment, histone bound non-pathogen-based nucleic acids can then be removed from the sample by use of a substrate which comprises an affinity agent that selectively binds to a histone protein, i.e., a histone-binding domain. Examples of affinity agents that can bind to a histone protein include, but are not limited to, chromodomain, Tudor, Malignant Brain Tumor (MBT), plant homeodomain (PHD), bromodomain, SANT, YEATS, Proline-Tryptophan-Tryptophan-Proline (PWWP), Bromo Adjacent Homology (BAH), Ankryin repeat, WD40 repeat, ATRX-DNMT3A-DNMT3L (ADD), or zn-CW. In another embodiment, the histone-binding domain can include a domain which specifically binds to a histone from a protein such as HAT1, CBP/P300, PCAF/GCNS, TIP60, HB01 (ScESA1, SpMST1), ScSAS3, ScSAS2 (SpMST2), ScRTT109, SirT2 (ScSir2), SUV39H1, SUV39H2, G9a, ESET/SETDB1, EuHMTase/GLP, CLL8, SpClr4, MLL1, MLL2, MLL3, MLL4, MLL5, SET1A, SET1B, ASH1, Sc/Sp SET1, SET2 (Sc/Sp SET2), NSD1, SYMD2, DOT1, Sc/Sp DOT1, Pr-SET 7/8, SUV4 20H1, SUV420H2, SpSet 9, EZH2, RIZ1, LSD1/BHC110, JHDM1a, JHDM1b, JHDM2a, JHDM2b, JMJD2A/JHDM3A, JMJD2B, JMJD2C/GASC1, JMJD2D, CARM1, PRMT4, PRMT5, Haspin, MSK1, MSK2, CMI, Mstl, Bmi/RinglA, RNF20/RNF40, or ScFPR4, or a histone-binding fragment thereof.

In additional embodiment, the disclosure also provides for a nucleic acid binding protein or nucleic acid binding domain that selectively binds to DNA that comprises a methylated CpG. CG dinucleotide motifs (“CpG sites” or “CG sites”) are found in regions of DNA where a cytosine nucleotide is followed by a guanine nucleotide in the linear sequence of bases along its 5′ to 3′ direction. CpG islands (or CG islands) are regions with a high frequency of CpG sites. CpG is shorthand for 5′-C-phosphate-G-3′, that is, cytosine and guanine separated by one phosphate. Cytosines in CpG dinucleotides can be methylated to form 5-methylcytosine. Cytosine methylation occurs throughout the human genome at many CpG sites. Cytosine methylation at CG sites also occurs throughout the genomes of other eukaryotes. In mammals, for example, 70% to 80% of CpG cytosines may be methylated. In pathogenic microorganisms of interest, such as bacteria and viruses, this CpG methylation does not occur or is significantly lower than the CpG methylation in the human genome. Thus, dehosting can be achieved by selectively cleaving CpG methylated DNA.

In some embodiments, the disclosure provides for a dehosting method which comprises a nucleic acid binding protein or binding domain which binds to CpG islands or CpG sites. In another embodiment, the binding domain comprises a protein or fragment thereof that binds to methylated CpG islands. In yet another embodiment, the nucleic acid binding protein binding domain comprises a methyl-CpG-binding domain (MBD). An example of an MBD is a polypeptide of about 70 residues that folds into an alpha/beta sandwich structure comprising a layer of twisted beta sheet, backed by another layer formed by the alpha1 helix and a hairpin loop at the C terminus. These layers are both amphipathic, with the alpha1 helix and the beta sheet lying parallel and the hydrophobic faces tightly packed against each other. The beta sheet is composed of two long inner strands (beta2 and beta3) sandwiched by two shorter outer strands (beta1 and beta4). In a further embodiment, the nucleic acid binding protein or binding domain comprises a protein selected from the group consisting of MECP2, MBD1, MBD2, and MBD4, or a fragment thereof. In yet a further embodiment, the nucleic acid binding protein or binding domain comprises MBD2. In a certain embodiment, the nucleic acid binding protein or binding domain comprises a fragment of MBD2. In another embodiment, the nucleic acid binding protein or binding domain comprises MBD5, MBD6, SETDB1, SETDB2, TIP5/BAZ2A, or BAZ2B, or a fragment thereof. In yet another embodiment, the nucleic acid binding protein or binding domain comprises a CpG methylation or demethylation protein, or a fragment thereof. In a further embodiment, CpG bound nonmicrobial nucleic acids can then be removed from the sample by use of a substrate which comprises an affinity agent that selectively binds to a nucleic acid binding protein or binding domain which binds to CpG islands or CpG sites. Examples of affinity agents include antibodies or antibody fragments that selectively bind to a nucleic acid binding protein or binding domain which binds to CpG islands or CpG sites. Affinity agents comprising antibodies or antibody fragments can be bound to a substrate or alternatively may itself be bound by a second antibody which is bound to a substrate, thereby providing a means to separate and remove the nonmicrobial nucleic acids from a sample.

In another embodiment the disclosure provides for dehosting method that uses a nuclease, or a recombinant protein which comprises a nuclease domain, whereby the nuclease cleaves non-pathogen-based nucleic acids into fragments. In the latter case, the recombinant protein may also comprise a nucleic acid protein binding domain having activity for nucleic acid binding proteins (e.g., histones, methyl-CpG-binding proteins). The nuclease or nuclease can include, but are not limited to, a non-specific nuclease, an endonuclease, non-specific endonuclease, non-specific exonuclease, a homing endonuclease, and restriction endonuclease. In another embodiment, the nuclease domain is derived from any nuclease where the nuclease or nuclease domain does not itself have its own unique target. In yet another embodiment, the nuclease domain has activity when fused to other proteins. Examples of non-specific nucleases include FokI and I-TevI. In some embodiments, the nuclease domain is FokI or a fragment thereof. In a further embodiment, the nuclease domain is I-TevI or a fragment thereof. In yet a further embodiment, the FokI or I-TevI or fragment thereof is unmutated and/or wild-type. Further examples of nucleases include but are not limited to, Deoxyribonuclease I (DNase I), RecBCD endonuclease, T7 endonuclease, T4 endonuclease IV, Bal 31 endonuclease, endonucleaseI (endo I), Micrococcal nuclease, Endonuclease II (endo VI, exo III), Neurospora endonuclease, S1-nuclease, P1-nuclease, Mung bean nuclease I, Ustilago nuclease (Dnase I), AP endonuclease, and Endo R.

As used herein, “Polymerase Chain Reaction (PCR)” refers to a process in which one or more nucleic acid molecules are amplified typically through the use of one or more primers under suitable amplification conditions. PCR is described in U.S. Pat. Nos. 4,683,195; 4,683,202; and 4,965,188; Saiki et al., 1985, Science 230:1350-1354; Mullis et al., 1986, Cold Springs Harbor Symp. Quant. Biol. 51:263-273; and Mullis and Faloona, 1987, Methods Enzymol. 155:335-350. The development and application of PCR are described extensively in the literature. For example, a range of PCR-related topics are discussed in PCR Technology—principles and applications for DNA amplification, 1989, (ed. H. A. Erlich) Stockton Press, New York; PCR Protocols: A guide to methods and applications, 1990, (ed. M. A. Innis et al.) Academic Press, San Diego; and PCR Strategies, 1995, (ed. M. A. Innis et al.) Academic Press, San Diego. Commercial vendors, such as ThermoFisher Scientific (Waltham, Conn.) market PCR reagents and publish PCR protocols.

PCR typically employs two oligonucleotide primers, commonly referred to in the art as a primer pair (a forward and reverse primer) that hybridize to a template nucleic acid (e.g., DNA or RNA molecule). Primers useful in some embodiments of the disclosure include oligonucleotides capable of acting as points of initiation of nucleic acid synthesis of a pathogen's genome or expressed polynucleotides (e.g., Zika virus nucleic acid sequences, e.g. one or more of the genome sequences provided in GenBank Accession numbers MF434516-MF434522 and MF801377-MF801426). Primers for PCR are typically single-stranded for maximum efficiency during amplification. Additionally, primers are often denatured, i.e., treated to promote linear, single-stranded primers in the amplification reaction. One method of denaturing primers is by heating (e.g., heating at 95° C. for 3-5 minutes). Although SEQ ID NOs: 1-96, 399-1562, 1563-3553, and 3554-7324 include both forward and reverse primers, a single primer from a particular table can be used in the methods of the disclosure so long as there is a second, e.g., universal primer or other primer oligonucleotide present.

If the template nucleic acid to be amplified is double-stranded, it is often needed to separate the two strands before it can be used as a template in PCR. Strand separation can be accomplished by any suitable denaturing methods known in the art including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions needed for denaturing template nucleic acids will depend, e.g., on the buffer salt concentration and the length and nucleotide composition of the nucleic acids being denatured, but typically ranges from about 90° C. to about 100° C. for a time depending on features of the reaction, such as but not limited to, melting temperature and nucleic acid length.

If the double-stranded template nucleic acid is denatured by heat, the reaction mixture is often allowed to cool to a temperature that promotes annealing of each primer to its target sequence. The temperature for annealing is usually from about 35° C. to about 65° C. (e.g., about 40° C. to about 60° C., about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase or reverse transcriptase is promoted or optimized, i.e., a temperature sufficient for nucleic acid extension to occur from the annealed primer to generate amplification products complementary to the template nucleic acid. The temperature should be sufficient to synthesize an extension/amplification product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; or about 60° C.). Extension times can be from about 10 sec to about 5 min (e.g., about 30 see to about 4 min; about 1 min to about 3 min; about 1 min 30 sec to about 2 min).

Since its inception, various amplification techniques have been described as variants or derivatives of PCR including, but not limited to, Ligase Chain Reaction (LCR, Wu and Wallace, 1989, Genomics 4:560-569 and Barany, 1991, Proc. Natl. Acad. Sci. USA 88:189-193); Polymerase Ligase Chain Reaction (Barany, 1991, PCR Methods and Applic. 1:5-16); Gap-LCR (PCT Patent Publication No. WO 90/01069); Repair Chain Reaction (European Patent Publication No. 439,182 A2), 3SR (Kwoh et al., 1989, Proc. Natl. Acad. Sci. USA 86:1173-1177; Guatelli et al., 1990, Proc. Natl. Acad. Sci. USA 87:1874-1878; PCT Patent Publication No. WO 92/0880A), NASBA (U.S. Pat. No. 5,130,238), Nested-Patch PCR (Varley and Mitra, (2008) Genome Research, 18:1844-50), asymmetric PCR (Wooddell & Burgess, (1996) Genome Research, 6:886-892), anchored PCR (Loh, (1991) Methods, 2, 1:11-19) inverse PCR (Ochman et al., (1988) Genetics, 120 (3):621-23), real-time quantitative PCR (Real Time-PCR) or quantitative PCR (qPCR) (Watson et al., (2004). Molecular Biology of the Gene (Fifth ed.). San Francisco: Benjamin Cummings), transcription based amplification system (TAS), strand displacement amplification (SDA), rolling circle amplification (RCA), hyper-branched RCA (HRCA) and Rapid Amplification of cDNA ends (RACE) (Lagarde et al., (2016), Nat. Comm., 7:1233. Additionally, digital PCR is a technique that that allows quantitative measurement of the number of target molecules in a sample. The basic premise is to divide a large sample into a number of smaller subvolumes (partitioned volumes), whereby the subvolumes contain on average a low number or single copy of target. By counting the number of successful amplification reactions in the subvolumes, one can deduce the starting copy number of the target molecule in the starting volume (U.S. Pat. No. 8,722,334).

Methods to reduce non-specific hybridization and amplification of off-target sequences have been improved through the application of “hot-start” techniques. A hot-start method typically involves an initial high (e.g., 95° C.-100° C.) incubation temperature step, after which one or more important reagents for amplification are added to the reaction mixture (e.g., MgCl₂ or deoxyribonucleotides (dNTPs)). By raising the reaction mixture temperature prior to the introduction of at least one amplification reagent a reduction in self-forming secondary structures, reduction in non-specific cross-linking, and a reduction in primer dimers can be achieved. Another method of reducing the formation of non-specific amplification products relies on heat-reversible inhibition of DNA polymerase by DNA polymerase-specific antibodies, as described in U.S. Pat. No. 5,338,671. The antibodies are incubated with a DNA polymerase in a buffer at room temperature prior to the assembly of the reaction mixture in order to allow formation of the antibody-DNA polymerase complex. Antibody inhibition of the DNA polymerase activity is inactivated by a high temperature incubation step prior to amplification.

Each cycle of PCR typically comprises three steps: denaturation, annealing, and synthesis; the method frequently involves about 15 to about 30 cycles and is routinely automated using a thermocycler. The steps of denaturation, annealing, and synthesis can be repeated as often as needed to produce the desired quantity of amplification products (e.g., corresponding to a required amount of target molecules). Often, the limiting factors in the amplification reaction are the amounts of primers, thermostable enzyme(s), and nucleoside triphosphates present in the reaction. The cycling steps (i.e., denaturation, annealing, and extension) are typically repeated at least once. The number of cycling steps will depend on the nature of the sample and/or the frequency of the target molecules in the sample. If the target molecule (e.g., Zika virus genome copies or other pathogen genome) is present in low numbers in a complex mixture of nucleic acids (e.g., a blood sample from a host), more cycling steps may be required to amplify the target molecule to a point where the amount of amplified product is sufficient for detection by the method.

PCR allows for rapid and specific diagnosis of infectious diseases, including those caused by bacteria or viruses. PCR also permits identification of non-cultivatable or slow-growing microorganisms such as mycobacteria, anaerobic bacteria, viruses from tissue culture assays or animal models. Multiplex PCR (a set of primer that allow amplification of at least two targets (e.g., amplification of at least 2 different genes or sub-regions thereof) provides additional flexibility to detect multiple target pathogenic microorganisms in a single assay or reaction. Other applications of PCR include detection of infectious pathogenic microorganisms and the discrimination of non-pathogenic from pathogenic strains (Salis A., (2009). Applications in Clinical Microbiology. Real-Time PCR: Current Technology and Applications). Amplification products from PCR reactions can be identified via gel electrophoresis although typically most assays utilize real-time PCR, where the amplification product of the PCR reaction is monitored in each cycle of amplification (i.e., in real-time) through the use of a double-stranded fluorescent dye or labeled probe. For example, PCR in veterinary applications can be used to detect bacterial pathogenic microorganisms including, but not limited to, Brachyspiraspp, Chlamydophila abortus, Chlamydophila psittaci, Coxiella burnetii, avian Coxiella-like organism, Lawsonia intracellularis, Mycobacterium avium subsp paratuberculosis, different species of Mycoplasma, and Streptococcus equi subsp equi. Identification of pathogenic microorganisms across mammalian species is useful when addressing zoonotic or potentially zoonotic infections.

Nucleic acid amplification of the target molecule can be carried out using any suitable amplification method, such as, but not limited to, PCR and related methods. In particular embodiments, amplification of a portion of a gene or genomic region from a pathogen present in a sample can be performed by real-time amplification, such as real-time PCR or reverse transcription PCR (RT-PCR). DNA sequencing can also be carried out using any of the various DNA sequencing methods and sequencing platforms available in the art, such as, but not limited to Illumina Inc., Oxford Nanopore Technologies, Inc., Ion Torrent, Helicos Biosciences Corp., Fluidigm, Nimblegen, Roche Sequencing, and the like. Exemplary DNA sequencing methods are described in the Examples section.

As used herein, a “sequencing assay” refers to a method for determining the order of nucleotides in at least a part of a nucleic acid molecule. A well-known method of sequencing is the “chain termination” method first described by Sanger et al., PNAS (USA) 74(12): 5463-5467 (1977) and detailed in SEQUENAS™ 2.0 product literature (Amersham Life Sciences, Cleveland) and in European Patent EP-B1-655506. In essence, DNA to be sequenced is obtained (e.g., isolated from a cell or sample), rendered single stranded (denatured), and placed into four vessels. Each vessel contains components to amplify the DNA, which include a template-dependent DNA polymerase, a primer complementary to the initiation site of sequencing of the DNA to be sequenced and deoxyribonucleotide triphosphates for each of the bases A, C, G and T, in a buffer conducive for hybridization between the primer and the DNA to be sequenced and chain extension of the hybridized primer. In addition, each of the vessels contains a small quantity of one type of dideoxynucleotide triphosphate, e.g. dideoxyadenosine triphosphate (“ddA”), dideoxyguanosine triphosphate (“ddG”), dideoxycytosine triphosphate (“ddC”), dideoxythymidine triphosphate (“ddT”). In each vessel, the target DNA is denatured and hybridized with a primer. The primers are extended to form a primer extension product that is complementary to the target DNA (i.e., the template nucleic acid). When a dideoxynucleotide is incorporated into the extending polymer, the polymer is prevented from further extension (blocked). Accordingly, in each vessel, a set of extended polymers of specific lengths are formed which are indicative of the positions of the nucleotide corresponding to the dideoxynucleotide in that vessel. The extended primer products are evaluated, for example using gel electrophoresis, to determine the sequence of the new polymeric strands.

More recently, the Sanger technique has been surpassed by Next-Generation Sequencing (NGS) platforms. The NGS platforms include automated, massively parallel, high-throughput sequencing methods (see, for example, Illumina iSeq, HiSeq, MiSeq, & NextSeq, Ion Torrent PGM and Proton, Roche 454 Life Sciences, Applied Biosystems SOLiD, Oxford Nanopore Technologies MinION, GridION, and PromethION instruments, and other DNA sequencing platforms). Some of the NGS methods include labels for detection of target molecules (e.g., one, two, three, four, or all nucleotide types corresponding to incorporation of A, G, T, or C, are labeled). In other embodiments, one, two, three, or all nucleotide types are label-free (See, ion semiconductor sequencing, such as the Ion Torrent and DNAe sequencing platforms) such that polymerization or nucleotide incorporation is measured by hydrogen ion release, pyrophosphate release, or a combination thereof. Other examples of NGS techniques contemplated for use with the disclosure include metagenomic NGS, which typically includes “shotgun” based amplification of one or more regions of a target nucleic acid molecule, such as but not limited to bacterial or viral genomes. Typically, metagenomic sequencing involves analysis of genetic information obtained from a sample that contains a plurality of microorganisms, including uncultured organisms. Generally, metagenomic sampling involves sample collection, isolation of nucleic acid molecules of interest, DNA sequencing of the nucleic acid molecules of interest to obtain sequencing reads, alignment of the sequencing reads to a reference genome, and identification of nucleic acid molecules having a sequence similarity above a certain threshold to one or more microorganisms.

In one embodiment, NGS methods of particular interest include a library preparation and/or a sequencing library. For example, a sample can contain an RNA target of interest (e.g., a viral genome from the Zika virus). The sample may be treated with a DNA destroying reagent (e.g., DNase) to isolate RNA molecules of interest. The RNA molecules can be amplified using primers and any amplification method in the art (e.g., reverse transcriptase) to form cDNA molecules and optionally, first- and second-strand DNA synthesis based on the cDNA molecules to increase the amount of DNA molecules in the reaction, thereby forming a library preparation. In some instances, the library preparation can be further amplified using the same or preferentially, different primers to generate increased amounts of the amplified DNA molecules from the library preparation, thereby forming a sequencing library. The sequencing library (or the library preparation may be used with any appropriate sequencing platform and corresponding sequencing assay (e.g., input DNA applied to the sequencing platform, such as Illumina HiSeq).

Metagenomic next-generation sequencing (mNGS) is a promising candidate approach for broad-spectrum pathogen identification in clinical samples as nearly all potential pathogenic microorganisms—viruses, bacteria, fungi, and parasites—can be detected on the basis of uniquely identifying DNA and/or RNA shotgun sequences. This method has been successfully applied for clinical diagnosis of infectious diseases, outbreak surveillance by whole-genome viral sequencing, and pathogen discovery. Thus, mNGS can be a particularly useful diagnostic tool for addressing unknown outbreaks, as it does not require a priori targeting of pathogenic microorganisms that may suddenly emerge in a new geographic region, such as EBOV in West Africa. However, current issues related to cost, sequencing depth, and background contamination limit the accuracy of mNGS-based diagnostics relative to specific PCR testing. In particular, the lower throughput and resulting lower sensitivity of portable nanopore sequencers creates a barrier for routine deployment of the platform for diagnostic and surveillance purposes, especially for infections typically present in clinical samples at very low titers, such as ZIKV.

In a particular embodiment, the disclosure provides for the use of transposome-based sequencing methods to identify a taxon or taxa of pathogenic microorganisms in a sample. Such transposome-based sequencing methods are described in US2014/0162897; US2015/0368638; US2018/0245069; US2018/0023119; WO20122103545; WO20150160895; WO2016130704; WO2019028047; U.S. Pat. No. 9,574,226; EP3161152. The number of steps required to transform a target nucleic acid such as DNA into adaptor-modified templates ready for next generation sequencing can be minimized by the use of transposase-mediated fragmentation and tagging. This process, referred to herein as “tagmentation,” often involves modification of a target nucleic acid by a transposome complex comprising a transposase enzyme complexed with a transposon pair comprising a single-stranded adaptor sequence and a double-stranded transposon end sequence region, along with optional additional sequences designed for a particular purpose. Tagmentation results in the simultaneous fragmentation of the target nucleic acid and ligation of the adaptors to the 5′ ends of both strands of duplex nucleic acid fragments. Where the transposome complexes are support-bound, the resulting fragments are bound to the solid support following the tagmentation reaction (either directly in the case of the 5′ linked transposome complexes, or via hybridization in the case of the 3′ linked transposome complexes). In particular, by using transposase and a transposon end compositions described herein one can generate libraries of di-tagged linear ssDNA fragments or tagged circular ssDNA fragments (and amplification products thereof) from target microbial DNA (including double-stranded cDNA prepared from microbial RNA) for genomic, subgenomic, transcriptomic, or metagenomic analysis or analysis of microbial RNA expression (e.g., for use in making labeled target for microarray analysis; e.g., for analysis of copy number variation, for detection and analysis of single nucleotide polymorphisms, and for finding genes from environmental samples such as soil or water sources).

Described herein are methods, compositions, and kits for detecting the presence (or absence) of a particular taxon of pathogens, such as but not limited to, a bacterium or virus, in a sample. These methods are useful in the areas of diagnosis of pathogenic infections, epidemiology, and disease surveillance, among others.

The disclosure generally relates to primers and/or probes for use in a sequencing assay to obtain sequencing reads, alignment of a first portion of the sequencing reads against a first reference genome and alignment of a second portion of the sequencing reads against a second reference genome for a second pathogen, and determining if the first pathogen and/or the second pathogen are present in the sample based on the alignment of the sequencing reads from the sequencing assay. In some embodiments, the disclosure relates to the production of sequencing reads by reverse transcription polymerase chain reaction (RT-PCR) or quantitative reverse transcription polymerase chain reaction (RT-qPCR). In yet another embodiment, the disclosure relates to the detection of a low titer pathogen in the sample amongst an excess of host DNA and/or co-infection by another pathogen. In some instances, the method allows for the detection of a low titer pathogen (e.g., a low titer viral pathogen such as Zika virus) amongst a sample containing another pathogen also present in a low titer.

The disclosure generally relates to methods for detecting a taxon of pathogenic microorganisms in a sample, wherein the sample may also contain host DNA and/or one or more additional and different taxon of pathogens. In one embodiment, the disclosure generally relates to a method of detecting a particular taxon of pathogenic microorganisms in a sample, comprising, (a) obtaining a sample from (from the environment or a subject) to be screened for a particular taxon of pathogens; (b) applying a sequencing assay to the sample to obtain sequence reads, the sequencing assay including primers having lengths that are within a range of 11 bp to 17 bp, wherein at least a portion of the primers were identified in a species of the particular taxon of pathogens; (c) aligning a first portion of the sequencing reads to a first reference genome for the particular taxon of pathogens; (d) aligning a second portion of the sequencing reads to a second reference genome corresponding to a different taxon of pathogens; and (e) determining whether the particular taxon and/or the different taxon of pathogenic microorganisms is present in the sample based on the alignment of the first and second portion of the sequencing reads.

The sample analyzed by the methods provided herein can be any sample including, but not limited to, any type of clinical sample or any type of environmental sample. In some embodiments, the sample contains a cell, tissue, or a bodily fluid. In some embodiments, the sample is a liquid or fluid sample. In some embodiments, the sample contains a body fluid such as whole blood, plasma, serum, urine, stool, saliva, lymph, spinal fluid, synovial fluid, nasal swab, respiratory secretions, vaginal fluid, amniotic fluid, or semen. In some embodiments, the sample comprises cells or tissue. In some embodiments, cells, cell fragments, or exosomes are removed from the sample, such as by centrifugation or filtration. In some embodiments, the sample is a biological sample. In some embodiments, the sample may be an unprocessed sample (e.g., whole blood) or a processed sample (e.g., serum, plasma) that contains cell-free or cell-associated nucleic acids. In some embodiments, the sample is enriched for certain types of nucleic acids, e.g., DNA, RNA, cell-free DNA, cell-free RNA, cell-free circulating DNA, cell-free circulating RNA, etc. In one embodiment, the sample is processed to isolate nucleic acids or to separate nucleic acids from other cellular components or nucleic acids within the sample (e.g., DNA or RNA isolation). In some embodiments, the sample is enriched for pathogen-specific nucleic acids. In some embodiments, the sample is enriched for pathogen-specific nucleic acids that are present in retroviruses. In another embodiment, the sample comprises RNA or DNA from a subject infected with, or suspected of harboring an infectious pathogen.

In a preferred embodiment, the sample comprises target nucleic acids. The target nucleic acids refer to nucleic acids to be analyzed in the sample. In some embodiments, the target nucleic acids are cell-free nucleic acids. For example, the target nucleic acids may be cell-free DNA, cell-free RNA (e.g., cell-free mRNA, cell-free miRNA, cell-free siRNA), or any combination thereof. In certain cases, the cell-free nucleic acids are pathogen nucleic acids, e.g., nucleic acids from pathogenic microorganisms such as viruses, bacteria, fungi, algae, and eukaryotic parasites. In some embodiments, different types of nucleic acids are present in the sample at the same time (e.g., host DNA or RNA and pathogen DNA or RNA).

In some embodiments, the sample is from a human subject, especially a human patient. In some embodiments, the sample may also be from any other type of subject including any plant, mammal, non-human mammal, non-human primate, domesticated animal (e.g., laboratory animals, household pets, or livestock), or non-domesticated animal (e.g., wildlife). In some embodiments, the subject is a dog, cat, rodent, mouse, hamster, cow, bird, chicken, pig, horse, goat, sheep, rabbit, or monkey. In some embodiments, the sample is from an environment (e.g., a water source, soil, food source, household or office or hospital items) and the like.

In one embodiment, the sample contains a certain amount, titer or concentration of target nucleic acids. Target nucleic acids within a sample may include double-stranded (ds) nucleic acids, single stranded (ss) nucleic acids, DNA, RNA, cDNA, dsDNA, ssDNA, circulating nucleic acids, circulating cell-free nucleic acids, circulating DNA, circulating RNA, genomic DNA, exosomes, cell-free pathogen nucleic acids, circulating pathogen nucleic acids, or any combination thereof. As used herein, the term “cell-free” refers to the condition of the nucleic acids as they appeared in the subject before the sample was obtained from the subject. For example, circulating cell-free nucleic acids includes cell-free nucleic acids circulating in the bloodstream of the subject. In contrast, nucleic acids that are extracted from a solid tissue, such as a biopsy, are generally not considered to be “cell-free”.

The sample may be obtained by any means known in the art. For example, the sample may be obtained by syringe (such as a FNA), blood draw, or direct placement into a vessel (such as urine, semen, feces, sputum, etc.), by swab, aspiration and the like. In some embodiments, obtaining the sample can include one or more processes that refine, purify and/or isolate the sample from its original composition, such as, but not limited to, nucleic acid extraction kits (e.g., PureLink Viral RNA/DNA purification kit, ThermoFisher Scientific, Catalog No.: 12280050).

In one embodiment, the subject is a host organism (e.g., a human) infected with a pathogen, at risk of infection by a pathogen, or suspected of having a pathogenic infection. In some embodiments, the subject is suspected of having a particular infection, e.g., suspected of exposure to the Zika virus, bacterial pathogen etc. In other embodiments, the subject is suspected of having an infection of unknown origin. In some embodiments, a host is infected with more than one pathogen (e.g., a bacterial infection and co-infection with a virus, fungi or parasite). In some embodiments, a subject has been diagnosed with, or is at risk for developing symptoms associated with viral, bacterial or fungal infection. In some embodiments, the subject is healthy and the methods disclosed herein are used to confirm the absence of a pathogen in the subject. In some embodiments, the subject is susceptible or is at risk of a pathogenic infection (e.g., an immunocompromised patient, elderly patient, newborn infant, is situated or has recently visited a locale known to possess infected subjects). In one example, the subject from whom the sample is obtained includes a mammalian host. In a specific embodiment, the subject includes a human host.

In some embodiments, the methods (and associated compositions and kits) disclosed herein are useful for detecting the presence of a first taxon of pathogenic microorganisms present in a sample. In another embodiment, the methods (and associated compositions and kits) disclosed herein are useful for detecting the absence of a particular taxon of pathogenic microorganisms present in a sample. The methods allow for the detection of one or more pathogenic microorganisms in a sample. In one embodiment, the method includes detection of 2, 3, 4, 5, 6, 7, 8, 9, 10 or more, pathogenic microorganisms from a single sample. In another embodiment, the method includes detection of at least two different taxa of pathogenic microorganisms from a single sample, e.g., a sample from a human subject. In some embodiments, the method includes determining whether a first taxon of pathogenic microorganisms is present in the sample (for example, based on alignment of one or more amplified nucleic acids obtained during the sequencing assay against a reference genome of the first taxon of pathogens). In another embodiment, the method includes determining whether a first taxon of pathogen is absent from the sample (for example, based on alignment of one or more amplified nucleic acids obtained during the sequencing assay against a reference genome of the first taxon of pathogens).

The methods provided herein (and associated kits and compositions) can be used to detect a plurality of pathogenic microorganisms present in a single sample. In one embodiment, the method includes detecting at least one viral taxon in the sample. In another embodiment, the method includes detecting at least one viral taxon and one bacterial or fungal taxon in the sample (i.e., a co-infection). In yet another embodiment, the method includes detecting at least one viral taxon and one algae, prion, or parasitic infection in the sample (i.e., a co-infection). In one embodiment, the method includes detecting at least one bacterial pathogen in a sample. In any of the embodiments, the method can detect pathogenic microorganisms that are resistant to a particular therapy (e.g., an antibiotic treatment, antiviral treatment, antifungal treatment, algicide, etc.).

In one embodiment, the method also provides for the detection of one of more viral genera. An exemplary list of viral genera is provided in List 1. It will be apparent to one of ordinary skill in the art that the viral genera provided in List 1 should not be construed as exhaustive. In another embodiment, the method provides for the detection of one of more fungal genera. An exemplary list of fungal genera is provided in List 2. It will be apparent to one of ordinary skill in the art that the fungal genera provided in List 2 is not to be construed as exhaustive. In yet another embodiment, the method provides for the detection of one of more bacterial genera. An exemplary list of bacterial genera is provided in List 3. It will be apparent to one of ordinary skill in the art, that the bacterial genera provided in List 3 is not to be construed as exhaustive. It will be readily apparent that new bacterial, fungal and viral genera can be identified, e.g., based on sequence alignment of the new pathogen as compared against one or more known/existing pathogen taxa.

In one embodiment, the particular taxon of pathogenic microorganisms in a sample from a subject is a viral taxon. In some embodiments, the viral taxon includes viral genera and can include but is not limited to any one of the genera provided in List 1. In one embodiment, the viral genus is Flavivirus. Flavivirus is a genus of the family Flaviviridae which includes West Nile Virus (WNV), dengue virus, tick-borne encephalitis virus, Japanese encephalitis virus, yellow fever virus, Zika virus, insect-specific flaviviruses (ISFs) such as cell fusing agent virus (CFAV) Palm Creek virus (PCV) and Parramatta River virus (PaRV). Flaviviruses share significant common features such as size (40-65 nm), symmetry (enveloped, icosahedral nucleocapsid), and nucleic acids (positive sense single strand RNA of about 10,000 bases). Most Flaviviruses are transmitted by vectors such as a mosquitoes or ticks. Zika virus, yellow fever and dengue virus are frequently transmitted via mosquitoes and are capable of replicating in a host and transmitting viral material to other subjects even if the viral titer in the host is low. Other transmission routes for Flavivirus infection include blood transfusion, child birth, pregnancy, sexual contact, handling of infected animal carcasses or byproducts.

In another embodiment, the particular taxon of pathogenic microorganisms in a sample from a subject is the viral taxon. In some embodiments, the viral taxon includes the viral genera Alphavirus. Alphavirus is a genus of the family Togaviridae which includes Chikungunya virus (CHIKV), Barmah Forest virus, Mayaro virus, Ross River virus, Semliki Forest virus, Sindbis virus, Una virus, Eastern Equine encephalitis virus, Tonate virus, Western Equine encephalitis virus, O'nyong'nyong virus and Venezuelan equine encephalitis virus. Alphaviruses share significant common features such as size (˜40 nm diameter nucleocapsid), symmetry (enveloped, isometric nucleocapsid), and nucleic acids (positive sense, single strand RNA genome of about 11,000 bases). Alphaviruses are mainly transmitted by mosquitoes.

Viral titer, viral load or viral burden are used herein interchangeably and refer to a numerical expression of the quantity of a virus in a given volume. Viral titer frequently refers to the measurement of the lowest concentration of a virus that can successfully infect cells. To determine viral titer, typically, serial dilutions of a viral sample (containing a known amount of the virus) are prepared. For example, Zika virus can be prepared using petri dishes containing Vero cells (immortal cell line from kidneys of African Green monkeys) and a small amount of Zika virus added to the Vero cells (Contreras and Arumugaswami, (2016), J. Vis. Exp., (114), e54767). After several days, the Vero cells are evaluated to determine which, if any, of the serial dilutions experienced cell death and which, if any, of the serial dilutions continued to undergo cell replication. An exemplary technique for determining viral titer is the plaque assay (Dulbecco and Vogt, (1953) Cold Spring Harbor Symp. Quant. Biol., 18:273-79). Generally, the plaque assay includes preparing monolayers of cells incubated with a preparation of virus to allow adsorption of the virus into the cells. The cells are a covered with a nutrient layer such as agar to form a gel. When the infected cells release new progeny viral particles, the gel restricts the spread of viral particles to neighboring uninfected cells, which is visible as a circle or plaque in the petri dish. The viral titer from plaque assays is expressed as plaque forming units per ml (PFU/ml). Viral load, viral titer, etc., can be expressed as the number of viral particles or infectious particles per ml (e.g., viral genome copies per ml). For example, the quantity of virus per ml can be calculated by estimating the live amount of virus in a body fluid (e.g., RNA copies per ml of blood plasma). Tracking viral load is useful to monitor therapy e.g., treatment of chronic viral infections, patients who are immunocompromised or are recovering from organ/bone marrow transplantation.

In some embodiments, one or more other taxa of pathogen are identified that are distinct from the first taxon of pathogenic microorganisms against which the sample is screened. The sample can be screened for both pathogen taxa, although the first taxon of pathogenic microorganisms is typically present in the sample at a lower titer than the one or more other taxa of pathogens. In one embodiment, the one or more other taxa of pathogenic microorganisms includes a bacterial, fungal, algal, protozoan, and/or microscopic parasite. In one example, the one or more other taxa of pathogenic microorganisms is selected from any of the genera provided in List 2 and List 3.

The genome of an RNA virus, such as the Zika virus as well as other Flaviruses and retroviruses are comprised of ribonucleic acids (RNA). Accordingly, in order to perform PCR on the template nucleic acid, the RNA must first be transcribed into complementary DNA (cDNA) via the action of an RNA specific enzyme, reverse transcriptase prior to the sequencing assay. Reverse transcriptase uses the RNA template nucleic acid and a primer complementary to the 3′ end of the viral RNA to initiate synthesis of the first strand of complementary DNA (cDNA). This process is known as reverse transcription. The resulting synthesized nucleic acid molecule is cDNA, which by itself can be used directly as a template nucleic acid for amplification, such as PCR, for example using probes that are specific for at least a portion (e.g., between 10-50 nucleotides) of the cDNA sequence.

As disclosed herein, viral RNA obtained from a sample from a subject can be used in PCR assays to detect viral infection, among other pathogenic microorganisms or host DNA. The template nucleic acid (e.g., RNA or cDNA) need not be purified; it may be a minor fraction of a complex mixture (e.g., a clinical sample such as whole blood, biopsy, tissue sample or plasma). In some instances, the viral template nucleic acid can be present in low titer amounts, such as less than 100 infectious viral particles per mL. If needed or preferred, viral nucleic acid molecules may be extracted from a biological sample by routine techniques such as those described in Diagnostic Molecular Microbiology: Principles and Applications (Persing et al. (eds), 1993, American Society for Microbiology, Washington D.C.).

The methods (and associated kits and compositions) provided herein can be used to detect a taxon of pathogenic microorganisms in a sample from a subject (e.g., target nucleic acids) via a sequencing assay such as, multiplex RT-qPCR. The target nucleic acids can include, but are not limited to, whole or partial genomes, genetic loci, genes, exons, or introns. In one embodiment, the methods provided herein detect pathogenic target nucleic acids from a biological sample obtained from a subject. In some cases, the pathogenic target nucleic acids are present in complex clinical sample (e.g., an unprocessed sample such as whole blood or processed sample such as serum) containing nucleic acids from the subject (i.e., the host) and the pathogen. In some embodiments, the pathogenic target nucleic acids are associated with an infectious disease, such as Human Immunodeficiency Virus (HIV), Zika Virus, Hepatitis B, or Hepatitis C. In some embodiments the methods (and associated kits and compositions) are useful for the detection of pathogen nucleic acids transmitted via mosquitoes, such as Aedes aegypti and Aedes albopictus. In some embodiments, the pathogen target nucleic acids are viral nucleic acids. In another embodiment, the pathogen target nucleic acids are bacterial nucleic acids. In yet another embodiment, the target nucleic acids are viral nucleic acids present in a human sample. In a further embodiment, the target nucleic acids are Flavivirus nucleic acids present in a human sample such as urine or serum.

In some embodiments, the pathogen nucleic acids are present in a tissue sample, such as a tissue sample from a site of infection. In other embodiments, the pathogen nucleic acids have migrated from the site of infection; for example, it may be obtained from a sample containing circulating cell-free nucleic acids (e.g., circulating cf-DNA or cf-RNA).

In some embodiments, the target nucleic acids may make up a very small portion of the entire sample under evaluation, e.g., less than 1%, less than 0.5%, less than 0.1%, less than 0.01%, less than 0.001%, less than 0.0001%, less than 0.00001%, less than 0.000001%, or less than 0.0000001% of the total nucleic acids in the sample. In another embodiment, the target nucleic acids may make up from about 0.00001% to about 0.5% of the total nucleic acids in a sample. Often, the total nucleic acids in a sample may vary. For example, total cell-free nucleic acids (e.g., DNA or RNA) may be in a range of 1-100 ng/ml, e.g., (about 1, 5, 10, 20, 30, 40, 50, 80, 100 ng/ml). In some cases, the total concentration of cell-free nucleic acids in a sample is outside of this range (e.g., less than 1 ng/ml; in other cases, the total concentration is greater than 100 ng/ml). In another embodiment, total DNA in a sample (e.g., genomic, mitochondrial and pathogenic DNA extracted and purified from 100 μl of whole blood) may be in excess of 3 μg (see, Qiagen Dneasy Blood and Tissue purification kit, Catalog No. 69504). In some embodiments, the sample may contain a low viral titer of pathogen target nucleic acids which would still be elevated as compared to a non-infected, healthy sample. For example, pathogen target nucleic acids may make up less than 0.001% of total nucleic acids in an infected sample.

The length of target nucleic acids can vary. In some cases, target nucleic acids may be about or at least about 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000 or more nucleotides (or base pairs) in length, or a range of lengths between or including any two of the forgoing values (e.g., from about 30 to about 600 base pairs or nucleotides in length, from about 30 to about 250 base pairs or nucleotides in length, etc.). In some embodiments, the target nucleic acids are relatively short, e.g., less than 600 base pairs (or nucleotides) in length. In yet another embodiment, the target nucleic acids may be between 30 and 150 base pairs or nucleotides in length.

In some embodiments, the target nucleic acids include but are not limited to double-stranded (ds) nucleic acids, single stranded (ss) nucleic acids, DNA, RNA, cDNA, dsDNA, ssDNA, circulating nucleic acids, circulating cell-free nucleic acids, circulating DNA, circulating RNA, cell-free nucleic acids, cell-free DNA, cell-free RNA, circulating cell-free DNA, cell-free dsDNA, cell-free ssDNA, circulating cell-free RNA, genomic DNA, cell-free pathogen nucleic acids, circulating pathogen nucleic acids, circular DNA, circular RNA, circular single-stranded DNA, circular double-stranded DNA, or any combination thereof. The target nucleic acids are preferably nucleic acids derived from pathogenic microorganisms including but not limited to viruses, bacteria, fungi, parasites and other infectious microbes, including eukaryotic parasites. In some embodiments, target nucleic acids may be from the subject (e.g., host) as opposed to, or in addition to, target nucleic acids from a taxon of pathogens.

A sequencing library can be generated from a sample using the methods, compositions and kits provided herein or any suitable methods known in the art. Various commercial kits exist for the preparation of samples for NGS (e.g., Ion Ampliseq Library Kit 2.0, ThermoFisher Scientific, Catalog No.: 4475345). A sequencing library preferably comprises a plurality of target nucleic acids (e.g., a multiplex) that is compatible with any of the sequencing systems disclosed herein or known in the art. In some embodiments, a sequencing library generated from a sample from a subject is prepared for use on an Illumina sequencing platform (e.g., HiSeq or MiSeq). Optionally, target nucleic acids prepared for use in the sequencing library may comprise one or more adapters appended to one, or both, ends of the target nucleic acid molecules to aid in downstream analysis or classification. Optionally, the target nucleic acid molecules of the sequencing library may contain a barcode to distinguish one set of target nucleic acid molecules from a first sample from target nucleic acid molecules prepared from a second (e.g., a different sample from a different source or a sample collected at a different time from the same source (e.g., before and after infection) sample.

Steps for preparing a library preparation may include one or more of: obtaining (e.g., isolating or extracting) target nucleic acids from a sample, fragmenting the target nucleic acids, amplify the target nucleic acid using one or more primers thereby forming a library preparation, and storing the library preparation for later use. The library preparation steps outlined above are applicable to both DNA and RNA based libraries. Typically to amplify RNA, the target RNA is incubated with a DNA destroying reagent (e.g., DNase) to obtain an RNA sample. Steps for preparing a sequencing preparation may include one or more of: amplify the target nucleic acid molecules of the library preparation, attaching adapters to the amplified library preparation, and sequencing the amplified library preparation on a sequencing platform.

The methods (and associated compositions and kits) disclosed herein provide improved identification and/or quantification of target nucleic acid molecules in a sample from a subject, e.g., by RT-qPCR and/or NGS, particularly when the target nucleic acid molecules are present in low abundance in the sample (e.g., low viral titer) or when multiple pathogenic microorganisms are present. Additionally, the methods provided herein can be used to increase the yield of the target, particularly when the starting sample has relatively low amounts of the target.

Any detection method may be used which is suitable for the sequencing assay employed. In some embodiments, the sequencing assay can employ a label in the detection method. The term “label” as used herein refers to a composition detectable by spectroscopic, photochemical, biochemical, immunochemical, chemical, or other physical means. For example, useful labels include fluorescent dyes, luminescent agents, radioisotopes (e.g., ³²P, ³H), electron-dense reagents, enzymes, biotin, digoxigenin, or haptens and proteins, or other entities which can be made detectable, e.g., by incorporating a radiolabel into an oligonucleotide, peptide, or antibody specifically reactive with a target molecule. Exemplary detection methods include radioactive detection (e.g., ³²P), optical absorbance detection, e.g., UV-visible absorbance detection, optical emission detection, e.g., fluorescence or chemiluminescence. For example, labeled amplification products from a PCR, such as cDNA or DNA, can be detected using a sequencing platform by scanning all or portions of each labeled amplification product simultaneously or serially, depending on the sequencing platform and method used. For radioactive signals (e.g., ³²P), a phosphorimager device can be used (Johnston et al., 1990; Drmanac et al., 1992; 1993). In another embodiment, target molecules (e.g., cDNA molecules) can be label-free and their production detected by release of hydrogen ions during incorporation of each nucleotide during DNA synthesis (i.e., polymerization of DNA) (See, Ion Torrent sequencing platforms such as Personal Genome Machine and Proton sequencers, Life Technologies Corp., Carlsbad, Calif. and e.g., U.S. Pat. Nos. 9,139,874; 9,309,557 and 9,657,281). In another embodiment, the sequencing assay can include nanopore sequencing such as, but not limited to, sequencing methods disclosed in U.S. Pat. Nos. 8,852,864; 8,968,540; 9,121,059; 9,279,153; and 9,542,527.

In some embodiments, a signal from any of the detection methods utilized can be measured and/or analyzed manually or by appropriate computational methods to formulate results. The results can be measured to provide qualitative or quantitative results, depending on the needs of the user. Reaction conditions can include appropriate controls for verifying the integrity of amplification and/or sequencing assay, and for providing standard curves for quantitation, if desired (e.g., RT-qPCR). In some embodiments, a computational method comprises a computer system.

In some embodiments, the sequencing assay comprises a polymerase chain reaction (PCR). In one embodiment the sequencing assay comprises quantitative PCR (qPCR), reverse-transcription polymerase chain reaction (RT-PCR), or reverse transcription quantitative polymerase chain reaction (RT-qPCR).

In some embodiments, data obtained from the sequencing assay is in form of nucleotide sequences representing sequence reads obtained from the sample. In one embodiment, the sequencing assay comprises at least one reverse primer selected from any of the reverse primers in SEQ ID NOs: 1-96, 399-1562, 1563-3553, and 3554-7324. In another embodiment, the sequencing assay comprises at least one forward primer selected from any of the forward primers in SEQ ID NOs: 1-96, 399-1562, 1563-3553, and 3554-7324. In some embodiments, where the sample is suspected of containing an RNA virus, at least one of the primers in the sequencing assay comprises a primer that is complementary to either (i) the suspected RNA virus or (ii) another RNA virus. In another embodiment, where the sample is suspected of containing a bacterium, at least one of the primers in the sequencing assay comprises a primer that is complementary to the suspected bacterium or another bacterial genus. In some embodiments, the sequencing assay comprises reverse transcription of one or more target RNA molecules present in the sample using any of the primers set forth in SEQ ID NOs: 1-96, 399-1562, 1563-3553, and 3554-7324. In one embodiment, the sequencing assay further comprises a probe to determine the amount of amplified product produced in the sequencing assay by the primers. In one embodiment, the probe can include any one or more of the probes sequences provided herein as SEQ ID NO: 98-398. In one embodiment the amount of amplified product produced in the sequencing assay can be measured, determined or quantified by qPCR.

In some embodiments, the sequencing assay produces between 10,000 and 100 million raw sequencing reads. In some embodiments, the sequencing reads can be refined to remove bad quality or low-quality sequencing reads. In some embodiments, the sequencing assay provides greater than 10 sequencing reads and fewer than 100,000 sequencing reads per amplified target nucleic acid. In another embodiment, the sequencing reads can be deduplicated to remove duplicate reads from the raw sequencing assay data.

In some embodiments, a first portion of the sequencing reads are aligned against a first reference genome (e.g., for a particular taxon of pathogens). In another embodiment, a second portion of the sequencing reads are aligned against a second reference genome (i.e., a different taxon of pathogens). As used herein, “a first portion of the sequencing reads” generally refers to (i) a numerical value (e.g., at least 10 sequencing reads) or a percentage (e.g., at least 1%) of the total sequencing reads, or (ii) a nucleotide length within one or more of the sequencing reads that aligns with the first reference genome. In one embodiment, a first portion of the sequencing reads refers to at least 1%, 3%, 5%, 10%, 20%, 25%, or more, of the sequencing reads from the sequencing assay aligning against the first reference genome. In another embodiment, a first portion of the sequencing reads refers to an alignment of 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt, or a range that includes or is between any two of the foregoing nt values, within one or more of the sequencing reads aligned with the first reference genome. In another embodiment, aligning the first portion of sequencing reads comprises aligning between 10 and 50 contiguous nucleotides from one or more of the sequencing reads with the first reference genome. The first reference genome can comprise one or more viral, bacterial, fungal, algal, protozoan or parasitic genomes (or partial genomes thereof). In a preferred embodiment, the first reference genome is a viral genome. In one embodiment, the first reference genome comprises a consensus sequence for the taxon of pathogenic microorganisms (e.g., Flavivirus or Alphavirus). In some embodiments, the first reference genome comprises an arbitrary set of genomes for a single pathogen taxon (e.g., genomes from different species and/or individual strains within a particular taxon of pathogens) selected from one or more complete or partial genomes available in the art (e.g., GenBank Accession Numbers).

In some embodiments, a second portion of the sequencing reads are aligned against a second reference genome (e.g., for a different taxon of pathogens). As used herein, “a second portion of the sequencing reads” generally refers to (i) a numerical value (e.g., at least 10 sequencing reads) or a percentage (e.g., at least 1%) of the total sequencing reads, or (ii) a nucleotide length within one or more of the sequencing reads that aligns with the second reference genome. In one embodiment, a second portion of the sequencing reads refers to at least 1%, 3%, 5%, 10%, 20%, 25%, or more, of the sequencing reads from the sequencing assay aligning against the second reference genome. In another embodiment, a second portion of the sequencing reads refer to an alignment of 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt, or a range that includes or is between any two of the foregoing nt values, within one or more of the sequencing reads against the second reference genome. In another embodiment, aligning the second portion of sequencing reads comprises aligning between 10 and 50 contiguous nucleotides from one or more of the sequencing reads with the second reference genome. The second reference genome can comprise one or more known viral, bacterial, fungal, algal, protozoan or parasitic genomes (or partial genomes thereof). In a preferred embodiment, the second reference genome is a bacterial or fungal genome. In one embodiment, the second reference genome comprises a consensus sequence for the different taxon of pathogenic microorganisms (e.g., T2 bacteriophage). In another embodiment, the second reference genome comprises a consensus sequence obtained from between 10 and 200 genes present in the genome of the different taxon of pathogens. In some embodiments, the second reference genome comprises an arbitrary set of genomes (e.g., genomes from different species and/or individual strains within the different taxon of pathogens) for the different taxon of pathogenic microorganisms selected from one or more complete or partial genomes available in the art (e.g., GenBank Accession Numbers).

In one embodiment, the first reference genome is a complete or partial viral genome and the second reference genome is a complete or partial bacterial genome. In a preferred embodiment, a first portion of the sequencing reads are aligned against a first reference genome for a particular taxon of pathogenic microorganisms and a second portion of the sequencing reads are aligned against a second reference genome for a different taxon of pathogens, and based on the alignment of the first and second portion of the sequencing reads it is determined whether the particular taxon of pathogenic microorganisms is present or absent in the sample.

Any suitable method, calculation, or threshold may be used to determine whether the alignment of the first portion of the sequencing reads corresponds to the first reference genome. In one embodiment, the particular taxon of pathogenic microorganisms may be determined as present in the sample if at least 1%, 2%, 5%, 10% or more, of a first portion of the sequencing reads aligns with the first reference genome. Conversely, any suitable method, calculation or threshold may be used to determine whether a lack of alignment between the first portion of the sequencing reads and the first reference genome corresponds to a lack of the taxon of pathogenic microorganisms in the sample. For example, it may be determined that the target is absent from the sample, where greater than 95%, 96%, 97%, 98%, 99% or more of the sequencing reads do not align with the first reference genome.

Any suitable method, calculation or threshold may be used to determine whether the alignment of the second portion of the sequencing reads corresponds to the second reference genome. In one embodiment, the different taxon of pathogenic microorganisms may be determined as present in the sample if at least 1%, 2%, 5%, 10% or more, of a second portion of the sequencing reads align with the second reference genome. Conversely, any suitable method, calculation or threshold may be used to determine whether a lack of alignment between the second portion of the sequencing reads and the second reference genome corresponds to the different taxon of pathogenic microorganisms in the sample. For example, it may be determined that the different taxon of pathogenic microorganisms is absent from the sample, where greater than 95%, 96%, 97%, 98%, 99% or more of the sequencing reads do not align with the second reference genome.

The methods, compositions and kits disclosed herein contain primers that are useful for detection of pathogenic microorganisms in a sample. In some embodiments, the primers are suitable for the detection of a plurality of pathogenic microorganisms in a single sample. For example, two, three, four, five, or more primers or primer pairs, may be used in a single sequencing assay to determine whether a taxon of pathogenic microorganisms is present in the sample. In another embodiment, the primers or primer pairs may be used in a single sequencing assay to determine whether a plurality of pathogen taxa are present in a single sample. In some instances, each primer (or primer pair) is specific for an individual pathogen (e.g., species-specific or taxon-specific). In another embodiment, each primer (or primer pair) can be quasi-random sharing partial complementarity along the primer length to one or more species and/or individual strains from the particular taxon of pathogenic microorganisms or different taxon of pathogens. In another embodiment, the primers (or primer pairs) may be used to distinguish between different pathogenic microorganisms (e.g., distinguish bacterial pathogenic microorganisms from viral, fungal, algae or parasitic pathogens; or distinguish between taxa within a single taxonomic classification (e.g., bacterial domain or viral domain)). In one embodiment, at least one, two, three, four, or more of the primers in the sequencing assay are identified in a species of the particular taxon of pathogens. In another embodiment, at least one of the primers in the sequencing assay is identified in a species of the different taxon of pathogens. In some embodiments at least a portion of the primers in the sequencing assay are identified in a species of the particular taxon of pathogenic microorganisms (e.g., at least one of the primers or at least 5% of the primers) and is therefore indicative of that species (and the larger taxon) being present in the sample.

In contrast to traditional PCR methods, the primers disclosed herein are shorter in nucleotide length, in a range of between 11 to 17 nucleotides in length. Traditional PCR includes primers that are longer, for example between 18 and 30 nucleotides in length, and preferably between 20 and 24 nucleotides in length. Additionally, traditional PCR primers require that the primers are target-specific for the target(s) of interest to prevent random or mis-priming amplification products. Random primers have been used for amplification of nucleic acids molecules in general within a reaction mixture. The rationale for random primer use is that with enough random primers in the PCR method (e.g., hexamers or nonamers) all of the nucleic acid molecules in the reaction mixture have an equal likelihood of being amplified during the PCR process. Targeted primers (e.g., target specific primers) introduce bias in the PCR method because the targeted primers are selective for particular nucleic acid sequences within the sample and preferentially amplify the target nucleic acid sequences over a background of other nucleic acid molecules in the sample. In one aspect, the methods, compositions and kits disclosed herein comprise primers having a length of between 11 and 17 nucleotides. In some embodiments, a set (or panel) of primers or primer pairs for use with the disclosed methods, kits and compositions comprises at least two primers (one primer pair) and is preferably comprises more than 50, 100, 150, 200, 250, 300, 350, 400, 450, 500, 550, 600, 650, 700, 750, 800, 850, 900, 950, 1,000, 2,000, 3,000, 4,000, 5,000 primer pairs, or a range of primer pairs that includes or is between any two of the foregoing values.

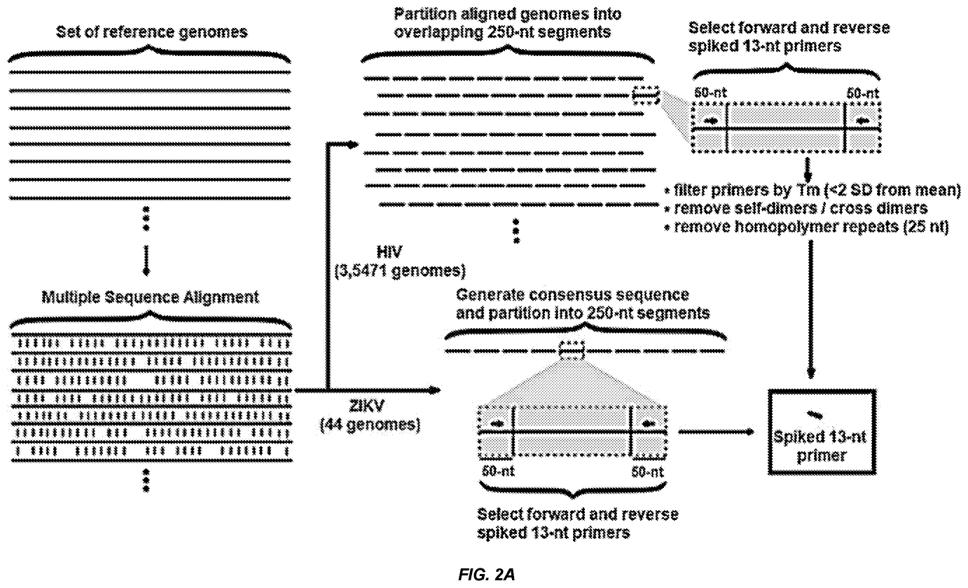

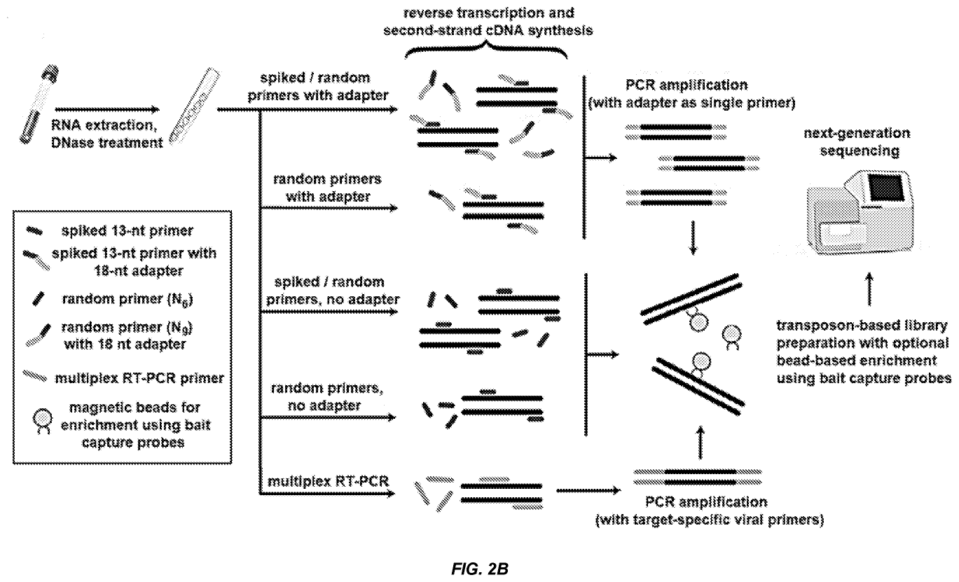

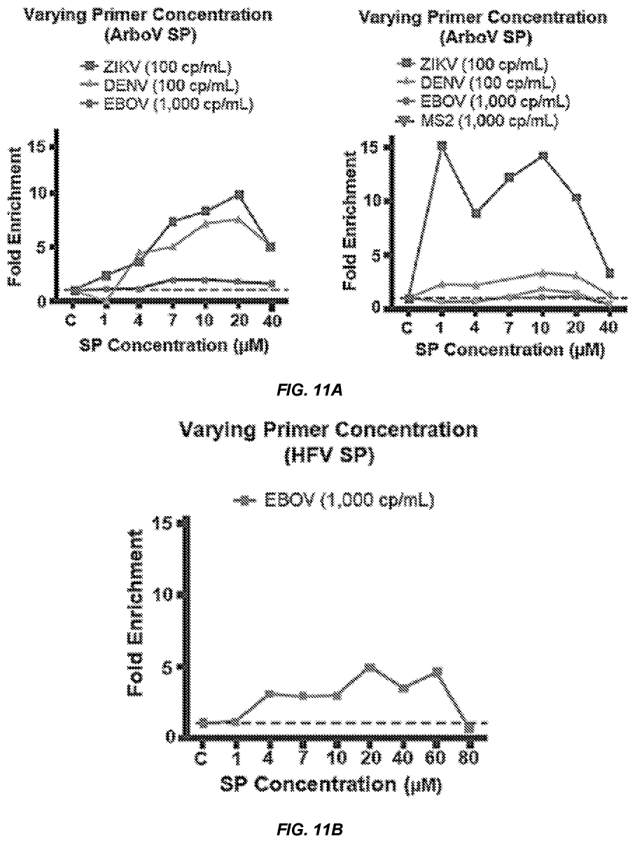

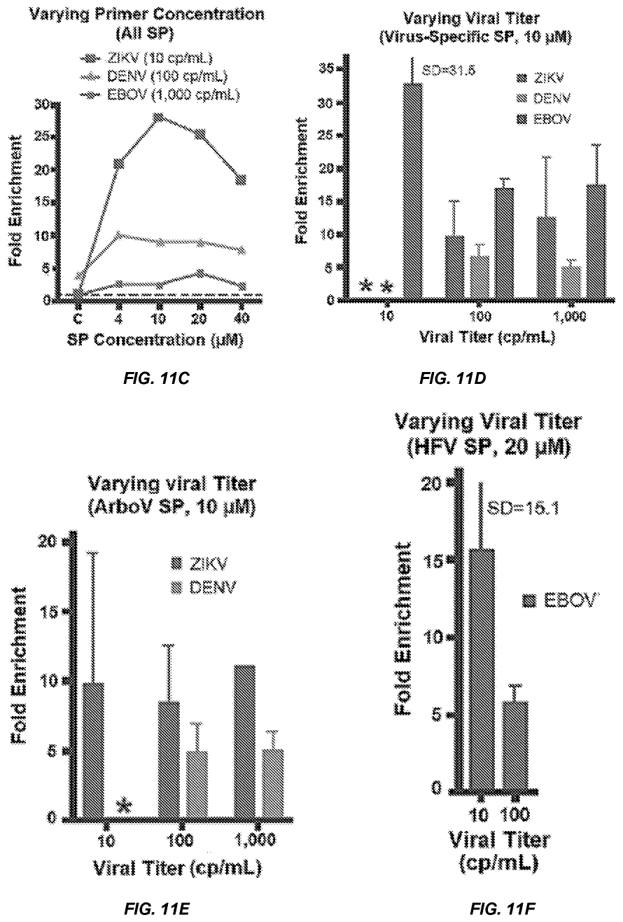

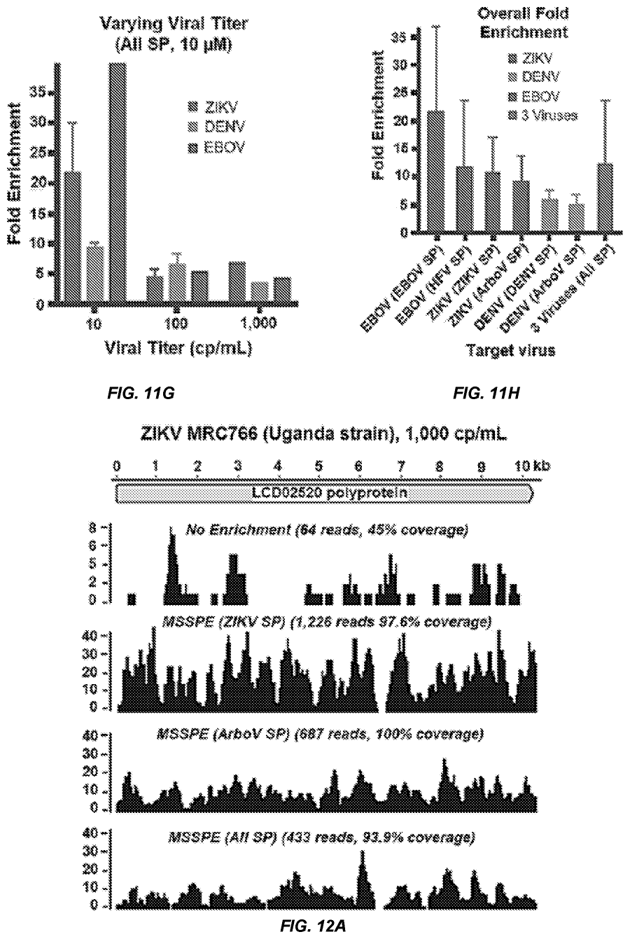

In one aspect, primers for use in the methods, compositions and kits are prepared according to the workflow provided in FIGS. 2A and 11A. In one embodiment, primers having a length of between 11 and 17 nucleotides are designed from a set of reference genomes (e.g., Zika virus genomes) by consecutive steps of multiple sequence alignment to form a consensus sequence, partitioning of the consensus sequence to form nucleotide segments, and selection of a forward and reverse primer (e.g., a primer pair) within a nucleotide window of a specified length present in the 5′ and 3′ terminal ends of the nucleotide segments. It will be apparent that the primers may be prepared by manual alignment, computational alignment, or any combination thereof.

In some embodiments, a computational algorithm can be used to design a set of primers from a set of reference genomes. In one embodiment, the algorithm can perform consecutive steps of multiple sequence alignment of the reference genomes to form a consensus sequence, partitioning of the consensus sequence into nucleotide segments of between 200 and 300 nucleotides, and selection of forward and reverse primers within the terminal ends of the nucleotide segment (e.g., select forward and reverse primers from within a 50 nucleotide window at each end of the nucleotide segment, see. FIGS. 2A and 11A). In another embodiment, the algorithm can perform consecutive steps of multiple sequence alignment of a set of reference genomes to generate a consensus sequence between the set of reference genomes, partitioning the consensus sequence into nucleotide segments of 100 bp, 200 bp, 300 bp, 400 bp, 500 bp, 600 bp, 700 bp, 800 bp, 900 bp, 1000 bp, 1200 bp, 1400 bp, 1500 bp, 2000 bp, or a range of base pairs that includes or is between any two of the foregoing values (e.g., 200-900, 300-800, 400-700, or 500-600 bp), and selection of forward and reverse primers within the terminal ends of each of the nucleotide segments (e.g., select one or more forward and reverse primers from within a 100 nucleotide window at each terminus of the nucleotide segment). In some embodiments, the algorithm can identify a single primer capable of amplifying at least one nucleotide segment across the reference genome. In one embodiment, the algorithm can identify a single primer capable of amplifying every nucleotide segment across the reference genome. In some embodiments, the algorithm can identify a single primer capable of amplifying each of the nucleotide segments across the reference genome, if present in the sample. In another example, the algorithm can design a primer capable of amplifying each species or individual strain of pathogen (e.g., Zika virus) presently known in the art based on alignment of the primer against the nucleotide segments (e.g., 600 base pair nucleotide segments) present in each of the species or individual strains of the pathogen (e.g., based on review of GenBank viral genome data). In another embodiment, the algorithm can design a primer such that it amplifies each species or individual strain of the Zika virus presently known, using a single nucleotide segment present in each of the species or individual strains of Zika virus. In another embodiment, a primer (e.g., primer 1) can be designed to amplify each species or individual strain of Zika virus presently known (e.g., based on review of GenBank genome data) based on predicted amplification of individual nucleotide segments across the Zika virus genome. In one embodiment, a primer panel of two or more primers can be designed, wherein Primer 1 does not amplify the same nucleotide segment as any other primer in the primer pool (e.g., Primer 2). In yet another embodiment, one or more primers within a primer panel can be designed in a redundant fashion, such that the genome of a pathogen of interest (e.g., Zika virus) is divided into nucleotide segments (e.g., between 200-300 bp or 600 bp) as disclosed above; designing a first primer based on the greatest (highest) number of nucleotide segments the primer can amplify across the pathogen's genome (preferably, across all species and individual strains) as compared to any other primer in the primer panel; and combining the first primer with a second primer, wherein the second primer is designed based on the next greatest (highest) number of nucleotide segments that primer 2 can amplify across the pathogen's genome, and so on. In this example, a plurality of primers can be identified that each amplify tens, or hundreds, of nucleotide segments across the pathogen's genome, preferably across all strains and individual species of the pathogen. In one embodiment, the primers are selected such that once a primer is predicted to amplify between 50 and 100 nucleotide segments across the pathogen's genome, those nucleotide segments are removed from the algorithm such that remaining primer designs do not amplify the removed nucleotide segments.

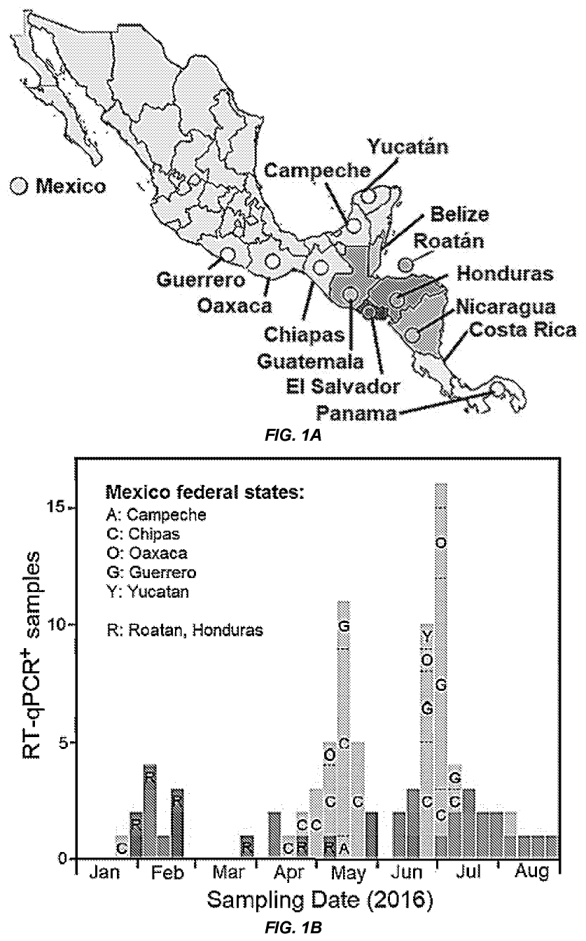

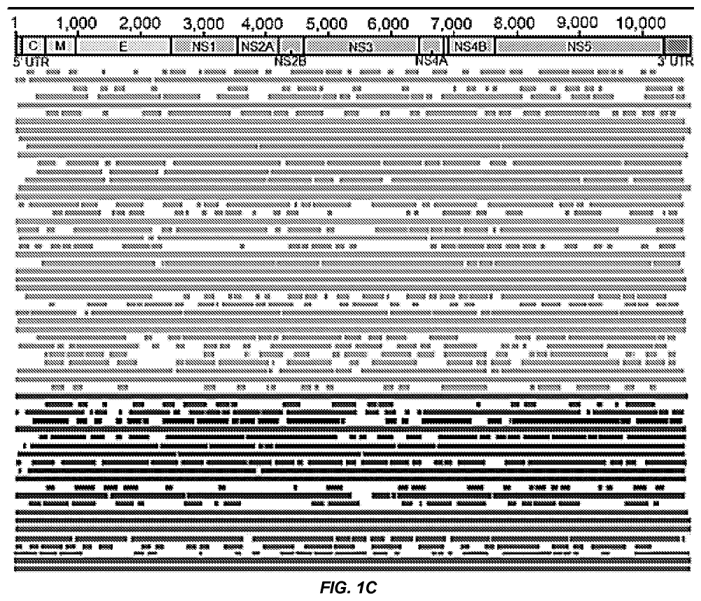

In one embodiment, the reference genomes are a set of arbitrary viral genomes from the same or different genera. In another embodiment, the reference genomes are a set of arbitrary bacterial genomes from the same or different genera. In yet another embodiment, the reference genomes are a set of arbitrary fungal, archaea or parasitic genomes from the same or different genera. It is contemplated that the reference genomes are selected in view of the type of pathogen to be detected. In one embodiment, the reference genomes are selected from genomes readily available in the art (e.g., GenBank), associated with a pathogen of interest (e.g., Zika virus), and optionally, associated with samples obtained from the same or adjacent geographic region (e.g., Central America, South America, North America, Mexico). The reference genomes do not require complete or full genomic coverage. As is evident from FIG. 1C, a consensus sequence for the Zika virus was prepared as outlined above from reference genomes that were incomplete. In some embodiments, it is preferred that greater than 25%, 50%, 75%, or more, of the reference genomes to utilize to generate a consensus sequence, which is partitioned into nucleotide segments, (e.g., of between 200 and 300 nucleotides or about 600 bp in length). In one embodiment, the nucleotide segments are approximately 250 nucleotides in length.

In some embodiments, primers are designed by partitioning the consensus sequence of the pathogen across a specific set of genes found in the genome of the pathogen. The consensus sequence for a particular taxon of pathogenic microorganisms (or different taxon of pathogens) represents highly conserved regions of the pathogen's genome found amongst different species or individual strains of a taxon of pathogens. Accordingly, by selecting highly conserved regions of the pathogen genome as the basis for developing primers, the resulting primers can be targeted to the highly conserved regions of the genome of the pathogen of interest. In one embodiment, when assessing fungi and bacterial pathogens, the primers can be designed based on specific genes, such as between 5 and 500 genes found in the genome of the fungi or bacteria of interest, preferably between 10 and 200 genes, and most preferably between 10 and 50 genes. In another embodiment, partitioning of the consensus sequence of the taxon of pathogenic microorganisms can include generation of nucleotide segments based on gene type or function. For example, genes associated with DNA or RNA polymerase may be located across the genome of the pathogen of interest and the consensus sequence is prepared from these regions of the genome into nucleotide segments; followed by selection of forward and reverse primers from the terminal ends of the nucleotide segments. In another embodiment, the genome of the pathogen of interest is partitioned into nucleotide segments, and the nucleotide segments falling within the specific set of genes of interest (e.g., antibiotic resistance genes) are retained and the remainder of the partitioned consensus sequence is not used to design the forward and reverse primers.

The primers can be designed to be of any length between 10-50 nucleotides, but are typically between 1017 nucleotides in length (e.g., about 13 nt in length). In some embodiments, the primers (e.g., the 11-17 nucleotide length primers) are optionally tagged or ligated to a nucleic acid adapter (See, FIG. 2B). The nucleic acid adapter can comprise between 10 and 50 nucleotides, in some instances between 15 and 30 nucleotides, and typically between 15 and 20 nucleotides. In one embodiment the adapter is an 18-mer. In a specific embodiment, the adapter comprises or consists of SEQ ID NO:97. The adapter can optionally include one or more modified nucleotides/nucleosides or nucleotide analogs. However, the adapter typically retains conventional hydrogen base-pair bonding capabilities. In one embodiment, the nucleic acid adapter ligated or tagged to the primers, as outlined above, is itself used as a primer in a subsequent or downstream amplification process. Optionally, the adapter contains a unique barcode sequence that allows for differentiation of samples in a multiplex assay.

In some embodiments, the method, kits and compositions disclosed herein comprise one or more additional primers distinct from the primers of 11-17 nucleotides in length prepared according to the method described above (e.g., see, FIGS. 2A and 11A). These additional primers are can be random primers that are selected without regard to the pathogen of interest to be detected (e.g., random hexamers (N6) or random nonamers (N9)). In one embodiment, the additional primers are random primers having a length of less than ten nucleotides. The additional primers can optionally include one or more modified nucleotides/nucleosides or nucleotide analogs. However, typically the additional primers retain conventional hydrogen base-pair bonding capabilities. In some embodiments, the primers of the disclosure are designed to hybridize to a target sequence in the sample (e.g., particular taxon of pathogens) and are present in an excess as compared to any additional primers (e.g., in the amplification reaction). For example, the primers can be present in a 2:1, 3:1, 4:1, 5:1, 6:1, 7:1, 8:1, 9:1, 10:1, or greater ratio as compared to the additional primers (e.g., random primers). In one embodiment, the primers of the disclosure are present in a 5:1 ratio (e.g., forward primer ratio 5:reverse primer ratio 5:random primer ratio 1). In another embodiment, the primers of the disclosure are present in a 10:1 ratio (e.g., forward primer ratio 5:reverse primer ratio 5:random primer ratio 1).

In some embodiments, a sample is screened for a particular taxon of pathogenic microorganisms by incubating the sample with a set of primers having lengths that are within a range of 11-17 nucleotides (i.e., 11, 12, 13, 14, 15, 16, or 17 nucleotides) that are optionally ligated or tagged to a nucleic acid adapter under suitable conditions (e.g., hybridization and amplification conditions) such that a plurality of amplified target nucleic acid molecules are generated (e.g., cDNA or DNA molecules). The primers (see, e.g., SEQ ID NOs: 1-96, 399-1562, 1563-3553, and 3554-7324) are combined with PCR reagents under reaction conditions that induce primer extension. For example, primer extension reactions generally include KCl, Tris-HCl, MgCl₂, denatured template nucleic acid, primer, and a polymerase or reverse transcriptase. The PCR usually contains dNTPs, such as dATP, dCTP, dTTP, dGTP, or one or more analogs thereof.

In some embodiments, the method further comprises incubating the sample in the presence of one or more random primers that are optionally ligated or tagged with the same (or different) nucleic acid adapter. In one embodiment, the method comprises generating a complementary DNA (cDNA) sequence to a target nucleic acid molecule (which corresponds to a particular taxon of pathogens) by reverse transcribing the target nucleic acid molecule by hybridizing one or more of the primers to a complementary nucleic acid sequence present in the sample. In one embodiment, the method further comprises amplifying the cDNA molecules using a nucleic acid adapter in a subsequent amplification reaction. In another embodiment, the cDNA molecules can be directly sequenced using any sequencing assay known in the art to obtain sequencing reads.

In one embodiment, a sample is screened for a particular taxon of pathogenic microorganisms by incubating the sample with a set of primers having lengths that are typically within a range of 11-17 nucleotides (i.e., 11, 12, 13, 14, 15, 16 or 17 nucleotides), optionally ligated to a nucleic acid adapter, in the presence of one or more random primers, optionally ligated to the same nucleic acid adapter, thereby allowing the primers to hybridize to a complementary nucleic acid sequence in the sample; extending the primers in a template dependent manner thereby generating cDNA; and optionally amplifying the cDNA to obtain a sequencing library. In some embodiments, the sequencing library can be sequenced using any method available in the art to obtain sequencing reads. In one embodiment, the sequencing reads can be filtered to remove adapter nucleic acid sequences, low-quality and/or low-complexity sequences.

In some embodiments, the methods (and associated kits and compositions) comprise one or more probes. The term “probe” as used herein refers to a molecule (e.g., a protein, nucleic acid, aptamer, etc.) that interacts with or binds to a target. Non-limiting examples of molecules that specifically interact with or specifically bind to a target include nucleic acids (e.g., oligonucleotides or magnetic beads coated with oligonucleotides), proteins (e.g., antibodies, transcription factors, zinc finger proteins, non-antibody protein scaffolds, etc.) and aptamers. Binding typically indicates that the probe binds a majority of the target, assuming an appropriate molar ratio of probe to target. For example, a probe that binds a target molecule typically binds to at least ⅔ of the target molecules in a solution (e.g., 67%, 75%, 80%, 85%, 90%, 91%, 92%, 93%, 94%, 95%, 96%, 97%, 98%, 99%, or 100%). In another embodiment, a probe binds to a target molecule with at least 2-fold greater affinity than non-target molecules, e.g., at least 4-fold, 5-fold, 6-fold, 7-fold, 8-fold, 9-fold, 10-fold, 20-fold, 25-fold, 50-fold, or 100-fold greater affinity. One of skill will recognize that some variability will arise depending on the method and/or threshold of determining binding.

In some embodiments, any one or more probes, e.g., selected from SEQ ID NOs:98-398 can be used. In some embodiments, the probe can comprise one or more moieties that allow for fluorescent detection of the probe when bound to or interacting with the target. In some embodiments, one or more probes can be added to the sequencing assay optionally, after formation of cDNA molecules (e.g., library preparation) to “pull down” targets having a complementary nucleic acid sequence. In one embodiment, the probe is a bait capture probe (see, e.g., Penalba et al., Mol. Ecol. Res., (2014) 14:1000-10; and xGen target capture probes commercially available from Integrated DNA Technologies, Iowa) (see, e.g., FIG. 11B). In some embodiments, the probes allow for selective enrichment of the target molecules from the sample. In some embodiments, the probe can be attached to a magnetic bead and/or biotinylated.

The disclosure also contemplates compositions which are useful in practicing the disclosure. Such compositions may include one or more primers or probes disclosed herein. Optionally, the compositions may further include an adapter.

In one embodiment, the disclosure generally relates to a nucleic acid molecule for detecting a target sequence from a particular taxon of pathogenic microorganisms comprising (a) a primer that is complementary or substantially complementary to the target sequence, wherein the primer is between 11 and 17 nucleotides in length; and (b) a primer set forth in SEQ ID NOs: 1-96, 399-1562, 1563-3553, and 3554-7324. In some embodiments, the composition further comprises an adapter located 5′ of the primer. In one embodiment, the adapter comprises or consists of SEQ ID NO:97.

In some embodiments, a composition comprising a reaction mixture containing at least one of the primers set forth in SEQ ID NOs: 1-96, 399-1562, 1563-3553, and 3554-7324 and a target sequence is also contemplated.

The disclosure also contemplates kits which are useful in practicing the disclosure. Such kits may include one or more primers or probes as disclosed herein. Optionally, the kits may include additional primers, probes, instructions, or vessels for one or more components of the kit. The kit may also include buffers and any other reagents that facilitate the method.

In one embodiment, the disclosure provides a kit for detecting the presence of a pathogen in a sample from a subject based on the presence of a sequencing read derived from the sample. In some embodiments, a first portion of the sequencing read aligns with a first reference genome, which corresponds to a particular taxon of pathogens. In some embodiments, a second portion of the sequencing read aligns with a second reference genome, which corresponds to a different taxon of pathogens.

In one embodiment, the disclosure generally relates to a kit comprising at least one primer set forth in SEQ ID NOs: 1-96, 399-1562, 1563-3553, and 3554-7324. In one embodiment, the kit is based on the presence or absence of a target sequence (or complement thereof) corresponding to a nucleic acid sequence present in the genome of a particular taxon of pathogens. In some embodiments, the target sequence corresponds to a genomic region of at least one species from the particular taxon of pathogens. In one embodiment, the target sequence corresponds to a reverse transcriptase (RT) region of a gene present in the genome of a particular taxon of pathogens.

In some embodiments, presence of the taxon of pathogenic microorganisms is determined by amplifying a region of a gene from the particular taxon of pathogenic microorganisms using gene-specific primers having lengths that are typically within a range of 11-17 nucleotides, and aligning a first portion of the target sequence against a first reference genome, wherein the gene-specific primers are any of the primers set forth in SEQ ID NOs: 1-96, 399-1562, 1563-3553, and 3554-7324. In another embodiment, presence of the particular taxon of pathogenic microorganisms is determined by amplifying a region of a gene from the pathogen using gene-specific primers having lengths that are typically within a range of 11-17 nucleotides, and aligning a second portion of the target sequence against a second reference genome, wherein the gene-specific primers are any of the primers set forth in SEQ ID NOs: 1-96, 399-1562, 1563-3553, and 3554-7324. In yet another embodiment, presence of the particular taxon of pathogenic microorganisms is determined by amplifying a target sequence in the sample using primers having lengths that are typically within a range of 11-17 nucleotides, aligning a first portion of the target sequence against a first reference genome, and aligning a second portion of the target sequence against a second reference genome, wherein the primers are any of the primers set forth in SEQ ID NOs: 1-96, 399-1562, 1563-3553, and 3554-7324.

In one embodiment, the kit further comprises an adapter. In one embodiment, the adapter is positioned 5′ of the primer. In some embodiments, the adapter comprises or consists of SEQ ID NO:97. In one embodiment, the kit further comprises one or more additional primers and/or probes. In one embodiment, the additional primers can comprise a random hexamer or a random nonamer. In one embodiment, the one or more probes can be included. For example, one or more probes can comprise any one or more of the probes selected from SEQ ID NOS:98-398.

In another embodiment, absence of the particular taxon of pathogenic microorganisms can be determined using a set of primers using any of the primers set forth in SEQ ID NOs: 1-96, 399-1562, 1563-3553, and 3554-7324, amplifying nucleic acids in the sample, and determining that none of the amplified nucleic acids align across a region of at least 10 to 50 nucleotides of a reference genome that corresponds to the particular taxon of pathogens.

In some embodiments, each of the primers is provided in a separate container, and the kit further includes an additional container having additional primers that are non-specific to the particular taxon of pathogenic microorganisms or different taxon of pathogenic microorganisms or random primers. In another embodiment, a solution or dry mix of pooled primers is provided in a single container, and the kit further includes additional primers (e.g., in the same or different container) that are non-specific to the particular taxon of pathogenic microorganisms or different taxon of pathogenic microorganisms or random primers.

In various embodiments, the kit includes at least one primer having a nucleotide sequence of comprising or consisting of any one of the primers set forth in SEQ ID NOs: 1-96, 399-1562, 1563-3553, and 3554-7324. In another embodiment, the kit includes at least two primers comprising or consisting of any of the primers set forth in SEQ ID NOs: 1-96, 399-1562, 1563-3553, and 3554-7324. In yet another embodiment, the kit comprises at least one forward primer as set forth in SEQ ID NOs: 1-96, 399-1562, 1563-3553, and 3554-7324 and at least one reverse primer comprising or consisting of a reverse primer set forth in SEQ ID NOs: 1-96, 399-1562, 1563-3553, and 3554-7324.

In one embodiment, the kit further comprises one or more probes, optionally residing in one or more vessels. In some embodiments, the one or more probes can be selected from any of SEQ ID NOS:98-398.

One aspect of the disclosure is oligonucleotides useful, e.g., as primers and/or probes, in the methods described herein. In various embodiments, an oligonucleotide of the disclosure has a nucleotide sequence consisting of sequence set forth in SEQ ID NOs: 1-96, 399-1562, 1563-3553, and 3554-7324.

Any of the computer systems mentioned herein may utilize any suitable number of subsystems. Examples of such subsystems are shown in FIG. 9 in computer apparatus 10. In some embodiments, a computer system includes a single computer apparatus, where the subsystems can be the components of the computer apparatus. In other embodiments, a computer system can include multiple computer apparatuses, each being a subsystem, with internal components.

The subsystems shown in FIG. 9 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art, such as serial port 77. For example, serial port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of instructions from system memory 72 or the storage device(s) 79 (e.g., a fixed disk), as well as the exchange of information between subsystems. The system memory 72 and/or the storage device(s) 79 may embody a computer readable medium. Any of the values mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present invention can be implemented in the form of control logic using hardware (e.g., an application specific integrated circuit or field programmable gate array) and/or using computer software with a generally programmable processor in a modular or integrated manner. As user herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer program product (e.g. a hard drive, a CD, or an entire computer system), and may be present on or within different computer program products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

### EXAMPLES

### Example 1

Aspects of the disclosure are illustrated in the following Examples. Efforts have been made to ensure accuracy with respect to numbers used (e.g., amounts, concentrations, percent changes, and the like) but some experimental errors and deviations should be accounted for. Unless indicated otherwise, temperature is in degrees Celsius and pressure is at or near atmospheric. It should be understood that these Examples are given by way of illustration only and are not intended to limit the scope of what the inventors regard as various aspects of the present disclosure.

From December 2015, serum samples from all suspected ZIKV cases detected through passive surveillance from the 35 Mexican Social Security Institute (IMSS) delegations nationwide (located in 32 Mexican states) were submitted for ZIKV diagnosis to the Central Laboratory of Epidemiology (CLE), IMSS in Mexico City. All cases met the following suspect case definition: a person of any age who present exanthema accompanied by two or more of the following symptoms: fever, headache, conjunctivitis, arthralgia, myalgia, edema, pruritus and retroocular pain plus living in or having travelled to, within two weeks of fever onset, an area endemic for Aedes aegypti or A. albopictus with confirmed cases within the locality. Using red cap tubes (without anticoagulant), 5 mL of peripheral blood were taken by venipuncture of the inside part of the elbow, from which 2 to 3 mL of serum were obtained and sent under refrigeration conditions (2-8° C.) to the Central Laboratory of Epidemiology of IMSS in compliance with International Air Transport Association (IATA) triple packaging standards. All samples were taken during the acute phase of the disease (0-5 days following symptom onset).

ZIKV diagnosis by qRT-PCR was made according to guidelines from the National Institute of Diagnosis and Epidemiological Reference (InDRE) of Mexico. Forward and reverse primers (ZIKV 1086 and ZIKV 1162c, respectively) and Carboxyfluorescein (FAM)-labelled probes (ZIKV 1107-FAM) were used.

Viral RNA was extracted from 200 μL of patient serum using the QiAmp Viral RNA Extraction Kit (Qiagen, Hilden, Germany). The presence of ZIKV RNA was evaluated using QuantiTec Probe RT-PCR kit (Qiagen). Each reaction consisted of 12.5 μL of 2× reverse transcription master mix, 0.5 μl of QuantiTect RT mix, 0.25 μL of each primer (1 μM final concentration), 0.154 of probe (0.15 μM final concentration), 6.35 μL of water and 5 μL of RNA. Using the Applied Biosystems 7500 Fast system (Applied Biosystems, Foster City, USA) reverse transcription was carried out at 50° C. for 30 mins followed by 95° C. for 10 minutes and 45 cycles of 95° C. for 15 seconds and 69° C. for 1 minute. A few ZIKV samples that were borderline positive in Mexico at the time of initial screening were subsequently found to be negative upon repeat testing immediately prior to sequencing.

ZIKV samples from Mexico were collected as part of the national epidemiological surveillance program of the Mexican Institute of Social Security, which is a branch of the Ministry of Health. Samples along with accompanying clinical and epidemiological data were de-identified prior to analysis, and are thus considered exempt from human subject regulations with waiver of informed consent according to 45 CFR 46.101(b) of the United States Department of Health and Human Services. From December 2015, serum and urine samples were obtained and provided by the California Department of Public Health (CDPH) from 31 returning travelers from Mexico and the Central American Isthmus (El Salvador, Guatemala and Honduras). An additional 6 samples from patients in Roatán, Honduras were provided by the Blood Systems Research Institute (BSRI). These samples were extracted from patients matching the above suspect case definition. Viral nucleic acids were extracted using the EZ1 Virus Mini Kit v2.0 (Qiagen), and RNA was reverse transcribed using Superscript III Reverse Transcription Kit (Invitrogen). Nucleic acid extracts were subjected to DNase treatment at 37° C. for 30 minutes using Turbo DNase (Thermo-Fisher Scientific) and Baseline-ZERO DNase (Epicentre), followed by qRT-PCR testing for ZIKV.

RNA integrity was assessed using RNA 6000 Pico kit on the Bioanalyzer (Agilent). A few ZIKV samples that were borderline positive at the CDPH at the time of initial screening were subsequently found to be negative upon repeat qRT-PCR testing immediately prior to sequencing.

ZIKV samples from the CDPH were de-identified prior to analysis and are considered exempt from human subject regulations. The 6 samples from Honduras were collected under protocols approved by the institutional review boards of the University of California, San Francisco, and Universidad Nacional Autonoma de Honduras. Patients were enrolled and blood collected after obtaining informed consent from patients or their surrogates (parental permission for minors).

From February 2016, children enrolled in the Nicaraguan Pediatric Dengue Cohort Study, a community-based prospective study of children 2 to 14 years of age that has been ongoing since August 2004 in Managua, Nicaragua, were screened for Zika virus infection. Participants present to the Health Center Sócrates Flores Vivas at the first sign of illness and are followed daily during the acute phase of illness. Acute and convalescent (˜14-21 days after onset of symptoms) blood samples are drawn for dengue, chikungunya and Zika virus diagnostic testing. The case definition for dengue or Zika virus infection for children presenting with an undifferentiated febrile illness or rash with one or more of the following signs and symptoms: conjunctivitis, arthralgia, myalgia, and/or periarticular edema regardless of fever. All suspected Zika cases were confirmed by qualitative RT-PCR of viral RNA in serum and/or urine using triplex assays that simultaneously screen for DENV and CHIKV infections (ZCD assay, CDC Trioplex [https://] [www.fda.gov/MedicalDevices/Safety/EmergencySituations/ucm161496.htm#zika]) or in some cases the CDC ZIKV monoplex assay in parallel with a DENV-CHIKV multiplex assay.

The Institutional Review Boards of the Nicaraguan Ministry of Health and the University of California, Berkeley approved the study. Parents or legal guardians of all subjects provided written informed consent and subjects ≥6 years old provided assent.

Short 13-mer primers were designed using an in-house developed computational algorithm (FIG. 2A). Briefly, a multiple sequence alignment of the 44 ZIKV reference genomes available in the National Center for Biotechnological Information (NCBI) GenBank at the time of the design (March 2016) was performed using MAFFT software. The consensus sequence was then partitioned into 250-nt segments, followed by selection of forward and reverse 13-nt primers within 50-nt windows at the edges of each segment. Primers were designed according to the following criteria: (i) no degeneracy, (ii) no self-dimers or cross-dimers with hybridization ΔG<−9 kcal/mol, (iii) no homopolymer repeats >5 nt in length, and (iv) ranked by number of segments covered. Additional primers were designed manually at the 3′ end of the consensus sequence using Primer3. The complete 13-mer ZIKV primer set consisted of 45 forward and 51 reverse primers. Different concentrations of forward and reverse ZIKV primers were mixed with random hexamers at the reverse transcription stage to evaluate ZIKV sequence enrichment.

Metagenomic next-generation sequencing (mNGS) libraries were generated using Nextera XT kit (Illumina). Libraries were sequenced as 150 base pair (bp) paired-end runs on a HiSeq 2500 instrument (Illumina). Data was scanned for ZIKV reads using the SURPI (sequence-based ultra-rapid pathogen identification) computational pipeline and direct NCBI BLASTn alignment to ZIKV reference genome KJ776791 at an e-value threshold of 1×10⁻⁸. Consensus ZIKV genomes were assembled using the bwa-mem program, by mapping metagenomic ZIKV reads to reference genome KJ776791.

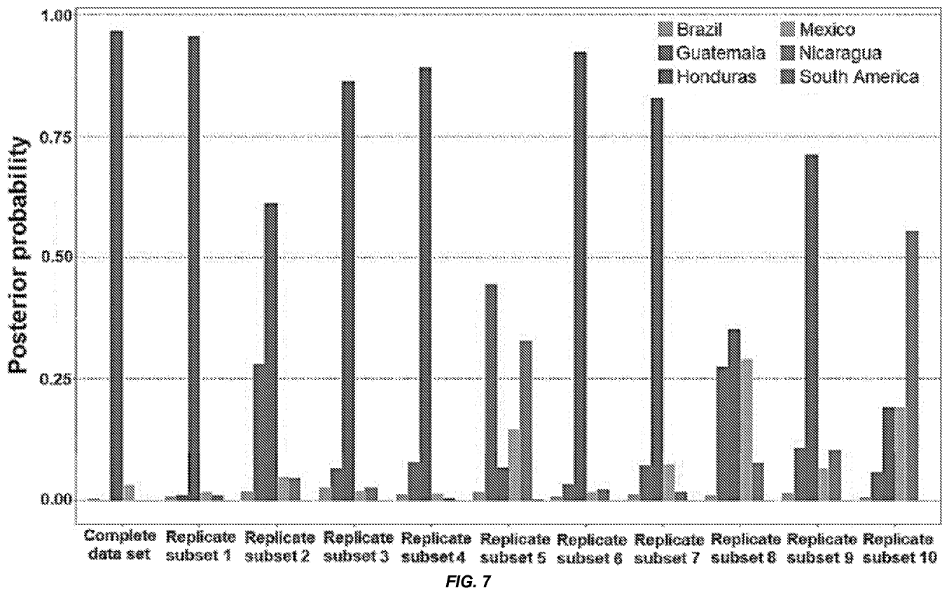

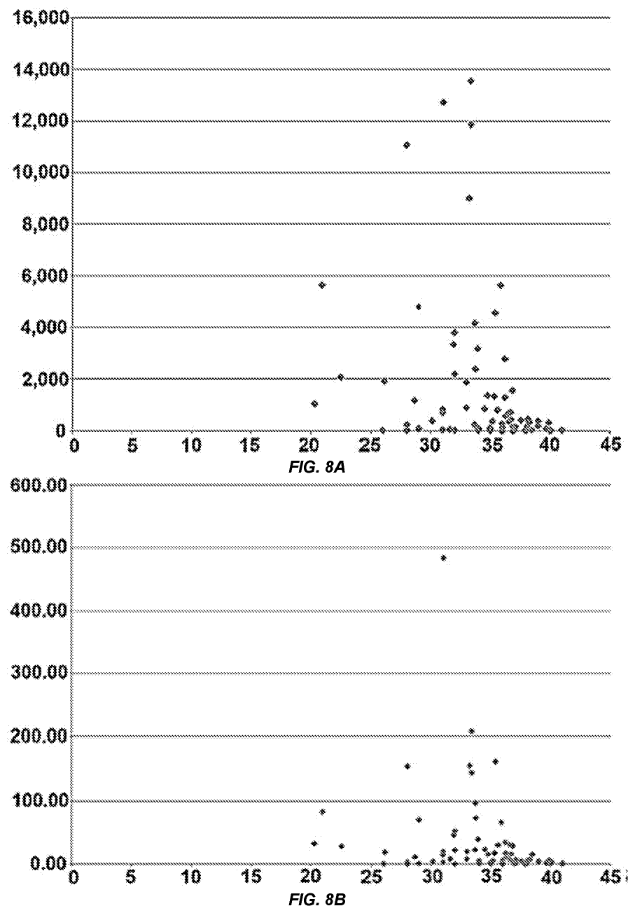

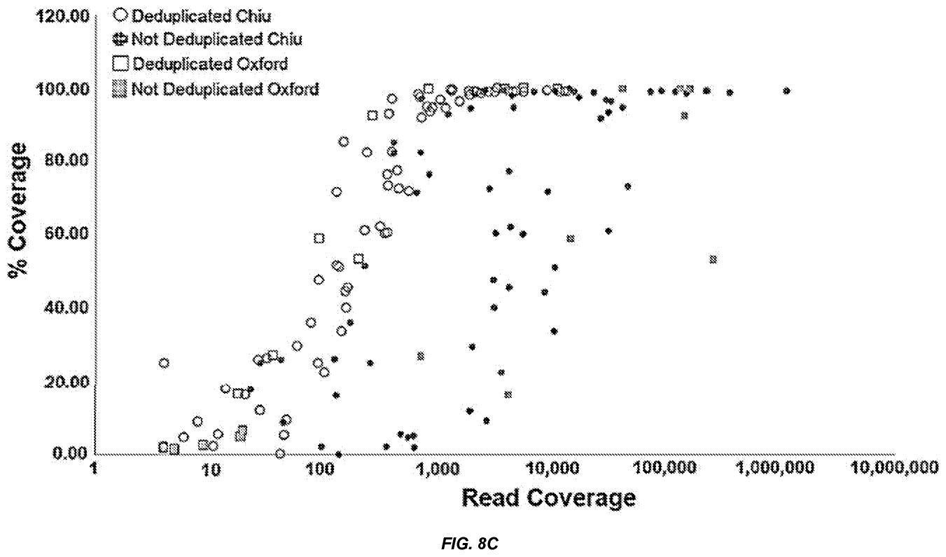

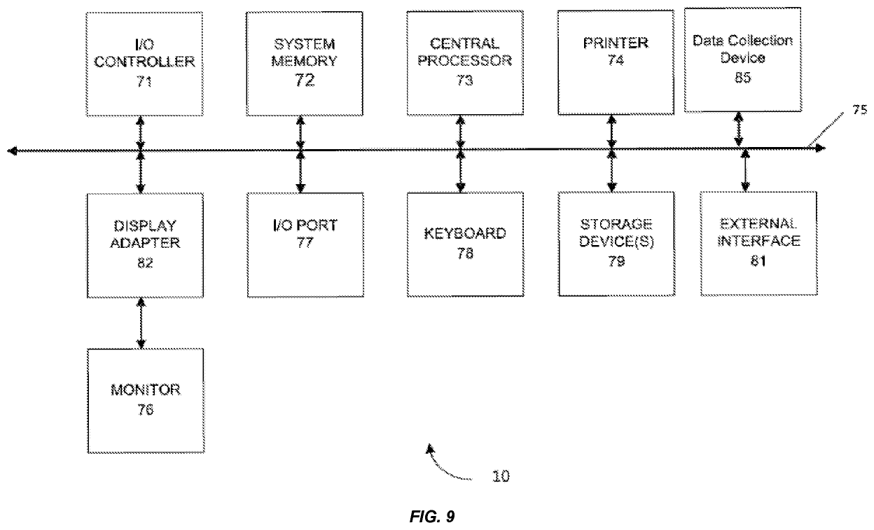

A subset of mNGS libraries were enriched for ZIKV sequences using xGen biotinylated lockdown bait capture probes (Integrated DNA Technologies) designed to tile across all 44 sequenced ZIKV genomes in GenBank as of March 2016 (FIG. 7). Capture probes were curated for redundancy at a 99% nucleotide similarity cutoff using cd-hit. Enrichment was performed on the mNGS libraries in pools of 8 libraries (including ZIKV-negative serum samples as controls) using the xGen lockdown probe protocol and the SeqCap EZ Hybridization and Wash Kit (Roche).

A subset of ZIKV infected serum collected from 14 subjects residing in Nicaragua were sequenced using a separate virus-specific-PCR free method previously described for Hepatitis C virus. Total RNA-seq libraries were prepared using the NEB Ultra Directional library kit with adaptations to the manufacturers protocol described elsewhere. By this method, RNA was heat-fragmented, reverse-transcribed using random hexamers then ligated to adapters that bind the manufacturers barcoded-PCR primers. Equal masses of amplified libraries are pooled for hybridization to a mixture of biotinylated 120mer oligonucleotides derived from 60 mer overlapping windows of the complete genome of the ZIKV strain KJ776791 (Integrated DNA Technologies) and captured with streptavidin-conjugated bead (Nimblegen) then PCR amplified to produce the final library for sequencing. The final library was sequenced using a MiSeq (Illumina) instrument using v3 chemistry producing 150 nt paired ends reads. Reads were mapped on KJ776791 reference genome using bwa-mem program to generate consensus genomes.

Published and available ZIKV coding sequences of the Asian genotype longer than 1500 nucleotides were retrieved from GenBank database as of June 2017. These 298 sequences were aligned together with the new ZIKV sequences generated here using MAFFT. A maximum likelihood (ML) phylogeny was estimated from this alignment using PhyM under a general time reversible nucleotide substitution model, with a gamma distributed among site rate variation and a proportion of invariant sites (GTR+Γ+I), as determined by jModelTest2. Statistical support for nodes of the ML phylogeny was assessed using a bootstrap approach with 100 replicates.

Temporal evolutionary signal in the alignment was evaluated using TempEst, which plots sample collection dates against root-to-tip genetic distances obtained from the ML phylogeny (see above). The plot indicated that the data set contained sufficient temporal signal for molecular clock analysis. Molecular clock phylogenies were estimated using the Bayesian MCMC approach implemented in BEAST v1.8.4. 4 independent runs of 100 million MCMC steps were computed, sampling parameters and trees every 5000 steps. An uncorrelated lognormal relaxed molecular clock model and a Bayesian skyline coalescent model were used; previous studies have demonstrated this combination to be the best fitting model combination for ZIKV in the Americas.

In each run, a SRD06 substitution model (Shapiro et al., 2006) was used, which employs a Hasegawa, Kishino and Yano nucleotide substitution model, a gamma distribution among site rate variation (HKY+Γ) and a codon position partition (positions (1+2) versus position 3). A non-informative continuous-time Markov chain reference prior (Ferreira and Suchard, 2008) was placed on the molecular clock rate for all analyses. The program Tracer v1.6 was used to check MCMC chain convergence and to compute marginal posterior distributions of parameters, after removal of 10% of the chain as burn-in. The program logcombiner was used to combine and subsample posterior tree distributions, after a 10% burn-in, thereby generating an empirical distribution of 1,500 molecular clock trees.

This empirical tree distribution was then used in subsequent phylogeographic analyses to infer ancestral branch locations using the Bayesian asymmetric discrete trait evolution model implemented in BEAST v1.8.4. Lineage movement events were counted among pairs of discrete locations using the robust counting approach. An in-house script was used to identify the earliest estimated ZIKV introductions into new locations from the results of the robust counting method. Viral lineage movement events were statistically supported (with Bayes factors >3) using the BSSVS (Bayesian stochastic search variable selection) approach, as implemented in BEAST version 1.8.4. TreeAnnotator was used to generate a summary maximum clade credibility (MCC) tree from the posterior distribution of trees (after removal of MCMC burn-in of 10%). The MCC phylogeny was drawn using the using ggtree package of the R software platform ([http://] [www.R-project.org/]). Box plots for node ages were generated using the ggplot2 package.

104 sequences comprising the Central American clade were analyzed using the serially sampled birth-death skyline model, implemented in BEAST2. 2 independent runs of 100 million MCMC steps were computed and sampled parameters every 10,000 steps. In each run, an uncorrelated lognormal relaxed clock model and a SRD06 substitution model were used, as in the phylogeographic analyses, above. An informative lognormal prior was placed on the molecular clock rate parameter, with mean equal to the median rate from the phylogeographic analyses and standard deviation set to include its 95% highest posterior densities (HPDs). A Laplace distribution was placed on the date of the MRCA with mean equal to the median estimated date in the phylogeographic analyses and scale parameter set to include its 95% HPDs. A lognormal prior with mean of 0 and standard deviation of 1.25 was placed on the effective reproductive number parameter (Rₑ). A Beta prior with α and β set to 1 and 999, respectively, was placed on the sampling proportion. The rate at which patients recover (becoming non-infectious rate) was fixed to 18.25, which corresponds to a mean infectious period of 20 days (this was based on the estimated mean generation time for ZIKV estimated by. The origin time of the Central American epidemic was bounded to be no older than Mar. 1, 2014. A lognormal prior with mean equal to Mar. 1, 2014 and standard deviation of 1 was also placed on the origin time.

The Rₑ parameter was allowed to change at 9 time points, equally spaced between the TMRCA and the time of the most recent sample. The sampling proportion parameter was assumed to be 0 before the time of the oldest sample and allowed to change at 9 time points, equally spaced between the oldest and most recent samples. The rate at which individuals become non-infectious rate was assumed to be constant through time. To assess the robustness of the estimates of Rₑ with respect to prior assumptions about the sampling proportion the above analyses was repeated with a sampling proportion prior favoring a lower sampling proportion (Beta distribution with α=1, β=9999) and a higher sampling proportion (Beta distribution with α=2, β=99).

The program Tracer v1.6 was used to check MCMC chain convergence and logcombiner was used to combine and subsample posterior distributions, after the removal of 25% of the chains as burn-in. Figures were produced using the R software platform using in-house scripts and the R-package bdskytools (available at [https://] [github.comiaduplessis/bdskytools]).

To predict for seasonal variation in the geographical distribution of the ZIKV vector Aedes aegypti in Central America a monthly A. aegypti suitability maps at a 5 km×5 km spatial resolution was used.

The high-resolution maps were aggregated at the country level. A linear regression model was then used to assess the correlation between monthly A. aegypti predicted climatic suitability and the number of weekly ZIKV notified cases, for each Central America country and for Mexico. This model tests how well vector suitability explains the variation in the number of ZIKV notified cases.

Genome sequences generated in this study are publicly available in GenBank database under the accession numbers: MF434516-MF434522 and MF801377-MF801426. Sequences of the primers and probes used in genome sequencing in this study are also available as SEQ ID NOs presented herein.

Serum and urine samples obtained from patients living in, or who had travelled to, Central America or Mexico and who exhibited symptoms consistent with ZIKV infection were screened for ZIKV by real-time quantitative reverse transcription PCR (qRT-PCR). A total of 95 specimens, sampled between January and August 2016, were qRT-PCR positive (59 from Mexico, 16 from Nicaragua, 9 from Honduras, 8 from Guatemala, 3 from El Salvador; FIGS. 1A, 1B). For 52 Mexico samples, the federal states where samples were collected were known (Campeche, Chiapas, Guerrero, Oaxaca, and Yucatan). Positive samples were collected, on average, 2 days after symptom onset, consistent with previous ZIKV studies in Brazil and Colombia. This period likely reflects the narrow 3-day overlap between ZIKV viremia (which persists for ˜9 days after infection) and the onset of symptoms (at ˜6 days after infection). The median cycle threshold (Ct) value of qRT-PCR positive samples was 36, similar to previous studies, and corresponded to a low RNA titer approaching the PCR detection threshold.

A general method of viral enrichment and genome recovery was developed from clinical samples using metagenomic next-generation sequencing (mNGS) for use in outbreak surveillance. The method is (1) applicable to any targeted virus, regardless of its representation in reference databases (e.g., from 1 to 10,000 genomes), (2) retains broad metagenomic sensitivity for the detection of novel or unexpected pathogens, or co-infections, (3) does not affect overall turnaround times for sample processing, and (4) sufficiently enriches metagenomic libraries to allow robust viral genome recovery from low-titer clinical samples. An automated computational algorithm was developed that takes an arbitrary set of reference genomes and designs a minimal panel of short, 13-nt spiked primers representing these genomes, to be added during the cDNA synthesis step of mNGS library preparation (reverse transcription followed by second-strand synthesis) (FIG. 2A). Following multiple sequence alignment and determination of the consensus sequence, the algorithm generated a set of 45 forward spiked primers and 51 reverse spiked primers for coverage of all 44 ZIKV genomes available in GenBank at the time of design (March 2016).

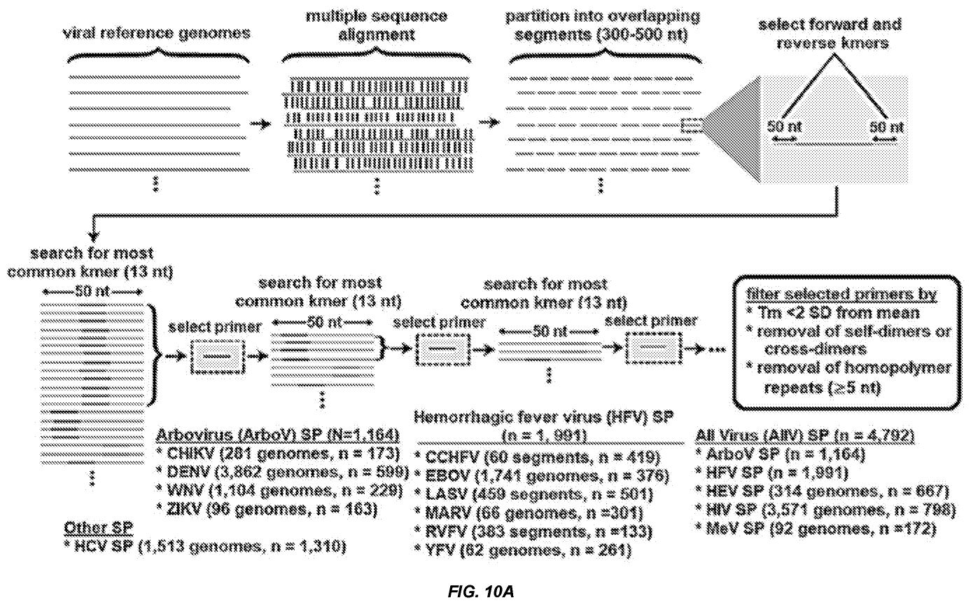

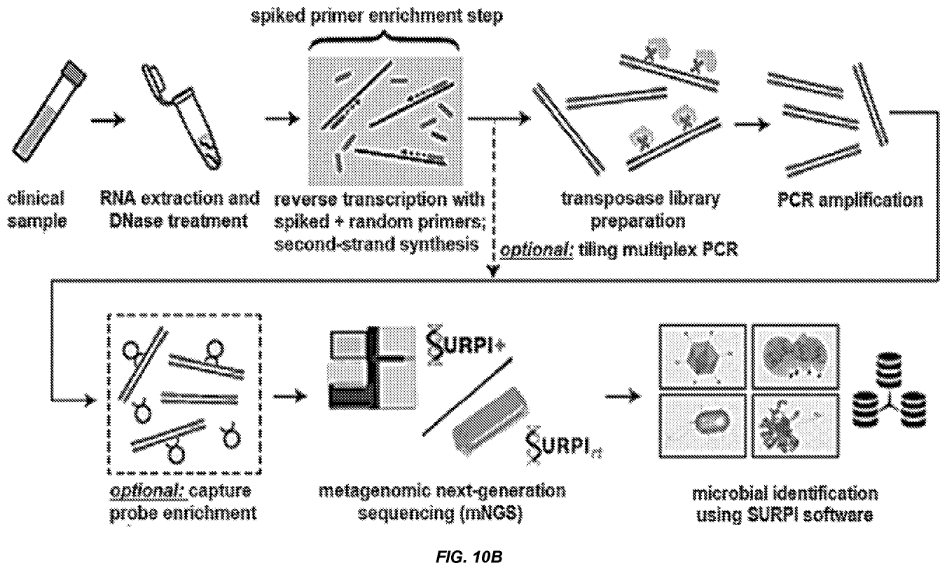

Different mixes of random hexamers and/or ZIKV spiked primers (see, FIG. 10) were tested at various ratios using serum samples containing ZIKV at titers of 100, 1,000, and 10,000 copies/mL. Spiked primers were tested with or without incorporation of an 18-nt adapter; the purpose of the adapter was to facilitate downstream PCR amplification using the adapter sequence as a single primer (See, Luk et al, Utility of Metagenomic Next-Generation Sequencing for Characterization of HIV and Human Pegivirus Diversity. PLoS ONE 10(11): e0141723, 2015, which discloses a primer having a random 9-mer linked to a specific 17-mer used to amplify the randomly primed library) and (FIG. 2B). To assess the impact of the spiked primer strategy on metagenomic detection of other infections or co-infections, human immunodeficiency virus, type 1 (HIV-1), hepatitis C virus (HCV), MS2 bacteriophage, and/or T1 bacteriophage were also added at predefined concentrations to the samples.

Using ZIKV-specific 13-nt spiked primers increased the number of ZIKV reads per million reads (RPM) in nearly all samples, compared to the ZIKV RPM obtained using random primers only; the magnitude of the increase was up to 9.4-fold and 8.9-fold (in metagenomic libraries) using spiked primers with or without an adapter sequence, respectively. Although the degree of enrichment using ZIKV spiked primers containing an adapter sequence was promising at moderate ZIKV titers of 1,000 and 10,000 copies/mL, enrichment was not observed at the lowest titer of 100 copies/mL. In contrast, increases in both RPM (4.9-fold and 8.3-fold increase) and percent genome coverage (1.47-fold and 2.47-fold) were noted at titers of 100 copies/mL using ZIKV spiked primers without an adapter. The greatest impact on genome coverage was seen using 13-nt reverse spiked primers (mixed at a 5:1 ratio of spiked ZIKV-specific to random primers), with 88.6% recovery of the genome at a ZIKV titer of 100 copies/mL, double the coverage obtained using random primers only.

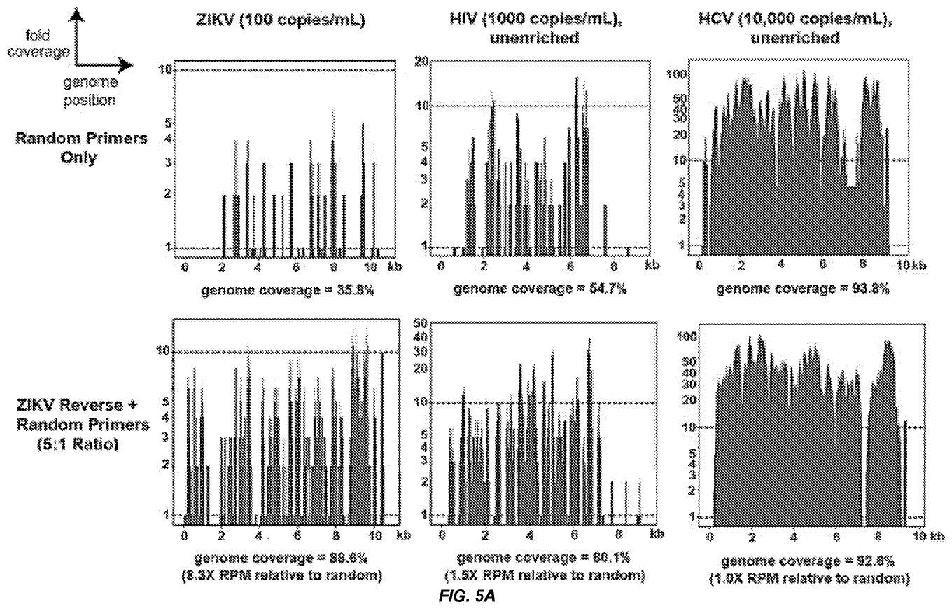

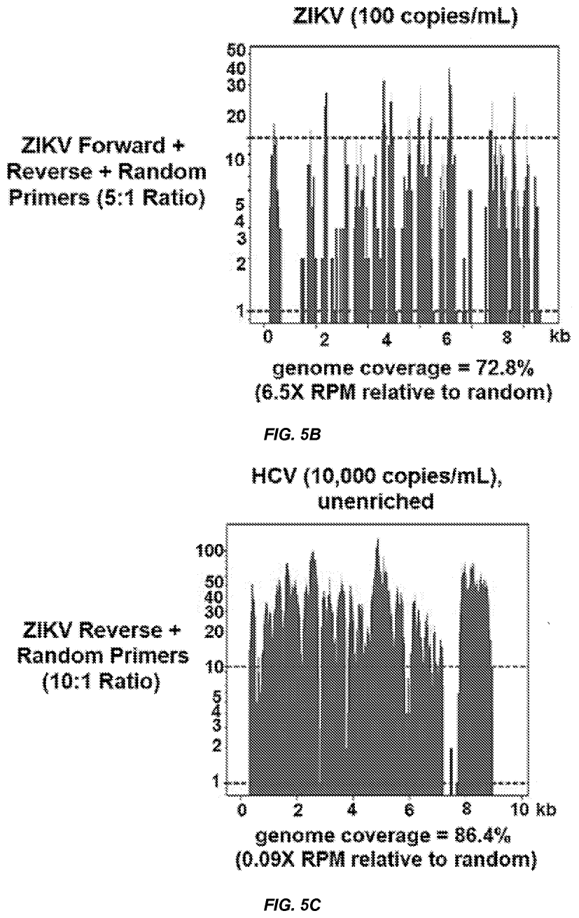

Notably, the addition of ZIKV spiked primers did not decrease the number of reads from untargeted RNA and DNA viruses in the samples, with the exception of MS2 bacteriophage, for which there was up to a 2-fold decrease in read counts. On average, however, an increase in RPM was observed, with more pronounced enrichment seen for HIV and T1 bacteriophage. Importantly, the genome coverage of HIV and HCV, both epidemic bloodborne pathogens, was not adversely affected by the use of ZIKV spiked primers (FIG. 5A). Other primer combinations tested such as, the use of forward in addition to reverse spiked primers (FIG. 5B), and a higher 10:1 concentration of spiked to random primers (FIG. 5C), were not found to improve overall coverage of ZIKV and untargeted viruses.

Using reverse 13-nt ZIKV spiked primers (the combination with the best ZIKV genome recovery as discussed above), reads that matched ZIKV in 71 of 81 samples were identified using mNGS. Coverage of the consensus ZIKV genomes generated from each sample ranged from 2% to 100%, with an average of 64%. Further probe enrichment for ZIKV genome recovery was attempted on 10 samples, whose original genome coverage ranged from 9 to 73%. Probe enrichment succeeded in all cases, with an average gain of 10% [0.1%-22%] genome coverage.

A further 14 Nicaraguan ZIKV samples were processed by a separate laboratory using an alternative mNGS method employing probe capture of metagenomic libraries without the use of spiked primers for reverse transcription. Coverage of the consensus ZIKV genomes generated from the Nicaraguan samples ranged from 1% to 100%, with an average of 47%.

Many sequenced samples had low genome coverage. Coverage was highly variable for samples with Ct values >30 and missing regions appeared to be randomly distributed across the ZIKV genome (FIG. 1C). To ensure a minimum level of information for phylogenetic analysis, only ZIKV sequences with genome coverage >30% were retained, resulting in a final dataset of 61 sequences with an average coverage of 82.6% (FIG. 1C).

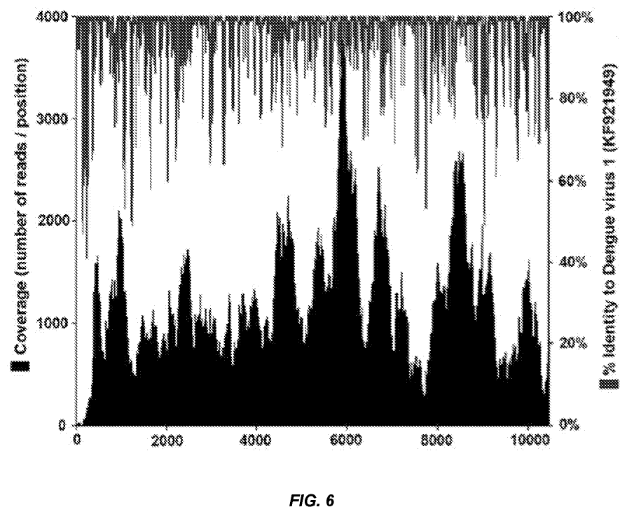

The mNGS approach enables concurrent detection of multiple pathogens. While performing mNGS analysis using ZIKV spiked primers for genome recovery, a dengue virus 1 (DENV1) infection was detected in a traveler returning from Tahiti with suspected ZIKV infection. Full DENV1 genome coverage was obtained directly from mNGS data, and the strain most closely matched isolates from Vietnam (see, FIG. 6). Four reads aligning totorque teno virus (TTV) in the family Anelloviridae were also identified in a ZIKV-infected patient from Mexico. TTV is not thought to be pathogenic to humans (Okamoto H. Curr. Top. Microbiol. Immunol., 331:1-20 (2009)). Reads matching Papillomaviridae were found in 42 of 81 samples and may be due to skin contamination introduced during sample collection. Contaminant viral reads matching known cultured viruses in the lab, such as human pegivirus 2, were also detected (data not shown).

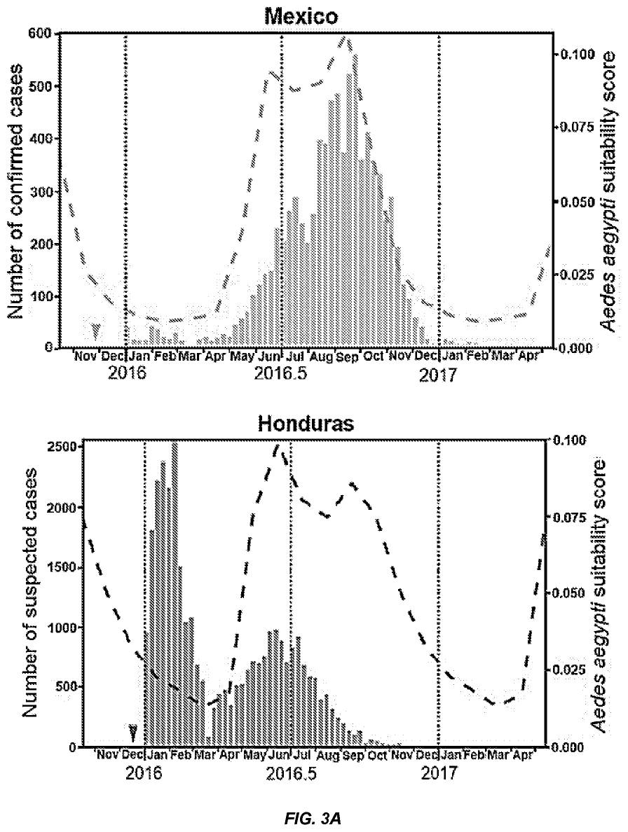

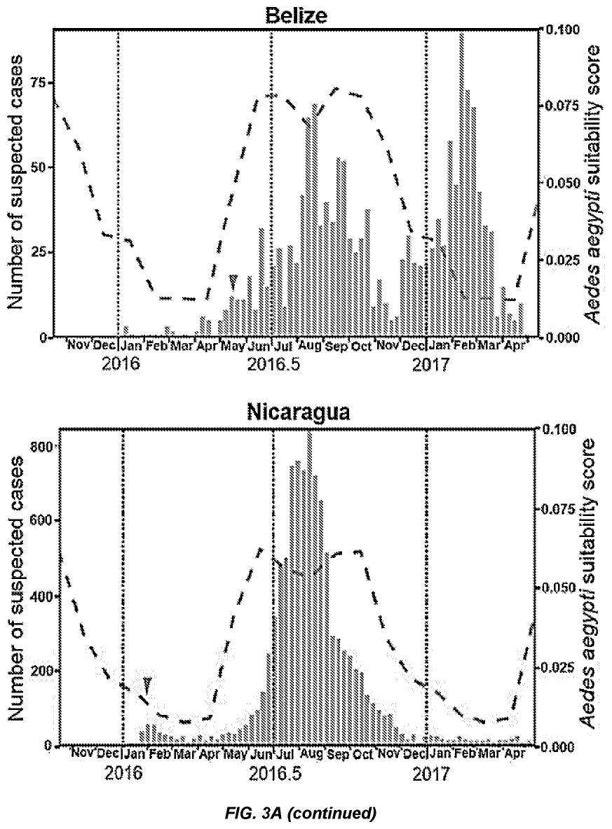

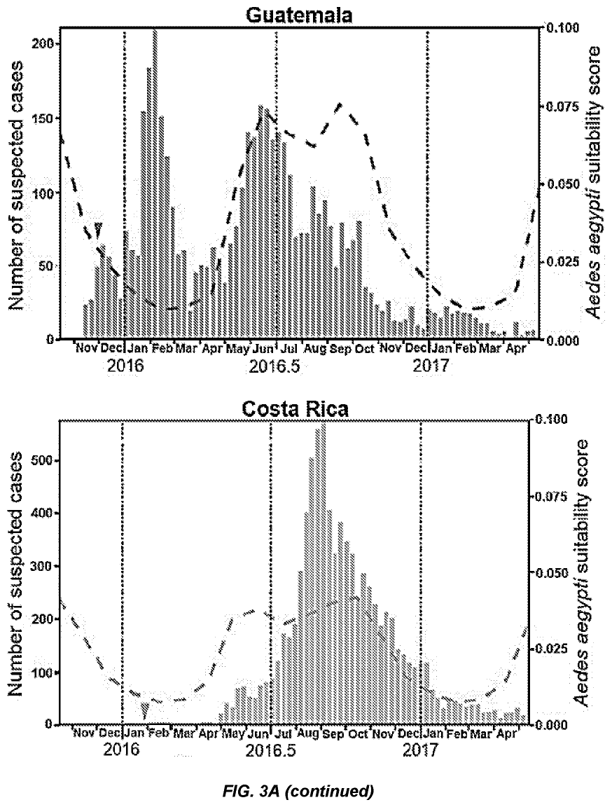

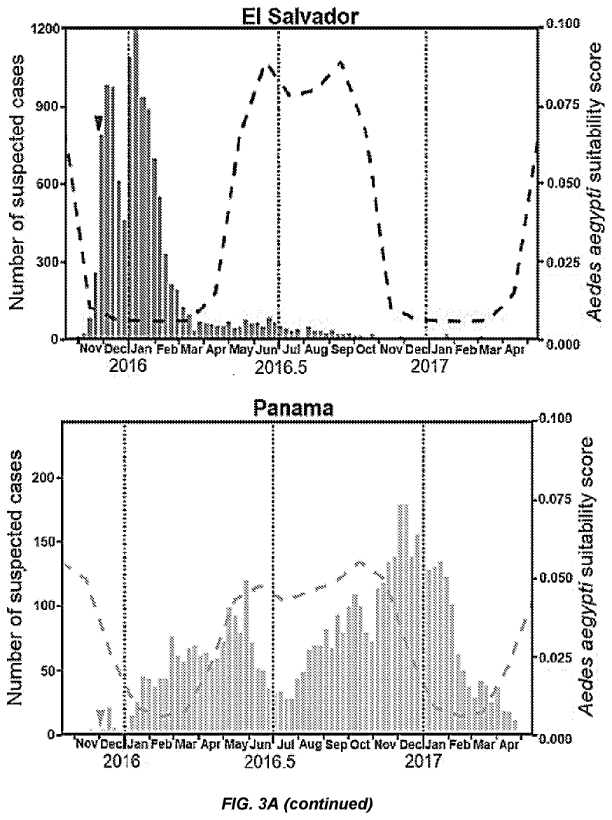

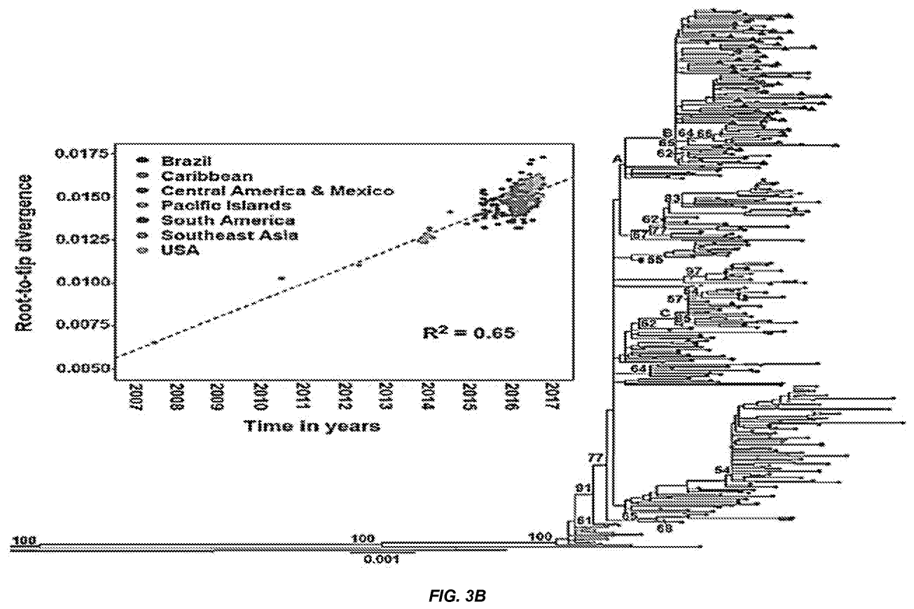

Weekly suspected ZIKV cases from Central American countries and confirmed cases for Mexico from 2015 to 2017 were extracted from Pan American Health Organization (PAHO) epidemiological reports (June 2017; FIG. 3). The date of first detection of ZIKV in each country ranged from November 2015 in El Salvador, to May 2016 in Belize. Countries reported a variety of epidemic trajectories. Costa Rica, Mexico and Nicaragua exhibited one epidemic in mid-2016, while two peaks in transmission were observed in Belize, Honduras and Guatemala. Suspected cases in El Salvador peaked only once, at the beginning of January 2016, while those in Panama showed no clear temporal pattern during 2016. These data should be interpreted cautiously because (i) case reporting will vary among countries, (ii) syndromic surveillance may not be able to distinguish between ZIKV and other viruses, such as DENV, with similar symptoms, and (iii) reporting intensity may vary through time, e.g. during national holidays.

To better understand these temporal patterns, each country was computed in order a measure the environmental suitability for the vector Aedes aegypti through time. The score was derived from monthly temperature, relative humidity, and precipitation data, as previously described. High climatic suitability scores were observed between May and October for most Central American countries (Belize, Guatemala, Honduras, El Salvador, Nicaragua) and Mexico. Honduras was found to have the highest average suitability (FIG. 3). Vector suitability scores in Costa Rica and Panama were typically lower and exhibited less seasonal variation. For Mexico, the suitability score represented only those 11 federal states that correspond to 95% of suspected ZIKV cases (Chiapas, Colima, Guerrero, Hidalgo, Morelos, Nuevo Leon, Oaxaca, Quintana Roo, Tabasco, Veracruz, and Yucatan).

A strong association between estimated vector suitability and weekly suspected ZIKV cases were observed for Mexico, Nicaragua and Costa Rica (R²>0.5; P<0.001; FIG. 3), as previously reported for Brazil. However, Belize, El Salvador, Guatemala, Honduras and Panama did not show such an association (R²<0.3; P>0.01; FIG. 3). Suspected cases peaked twice in Belize, Guatemala and Honduras, once between May and November (corresponding to the annual peak of mosquito suitability) and once between November and March. Unexpectedly, this latter rise in cases corresponded to low predicted vector suitability, and was also observed in El Salvador.

The sequence alignment used for phylogenetic analyses comprised the 61 ZIKV sequences generated here, plus 298 published and available sequences, as of June 2017. a maximum likelihood (ML) phylogeny with bootstrap node support values was first estimated (FIG. 3B). This tree revealed that 102 of the 107 ZIKV sequences from Central America and Mexico fell into a single monophyletic clade (clade B in FIG. 3B; bootstrap score=65%), which also contained two sequences from the USA. This Central America and Mexico clade was most closely related to ZIKV sequences from Brazil (clade A in FIG. 3B). Four ZIKV sequences from Panama and one from Mexico did not fall within clade B and were instead placed within a different clade (clade C in FIG. 3B; bootstrap score=85%). Within clade C, Panama sequences were most closely related to those from Colombia, whereas the Mexico sequence group was related to strains from Martinique. Thus, ZIKV had been introduced to Central America and Mexico (CAM) from other locations on multiple occasions, but most CAM infections descended from just one importation event (clade B).

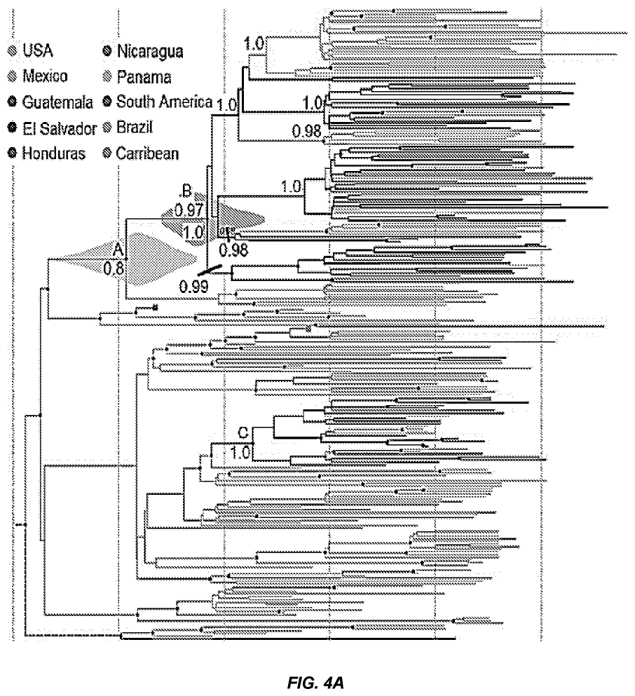

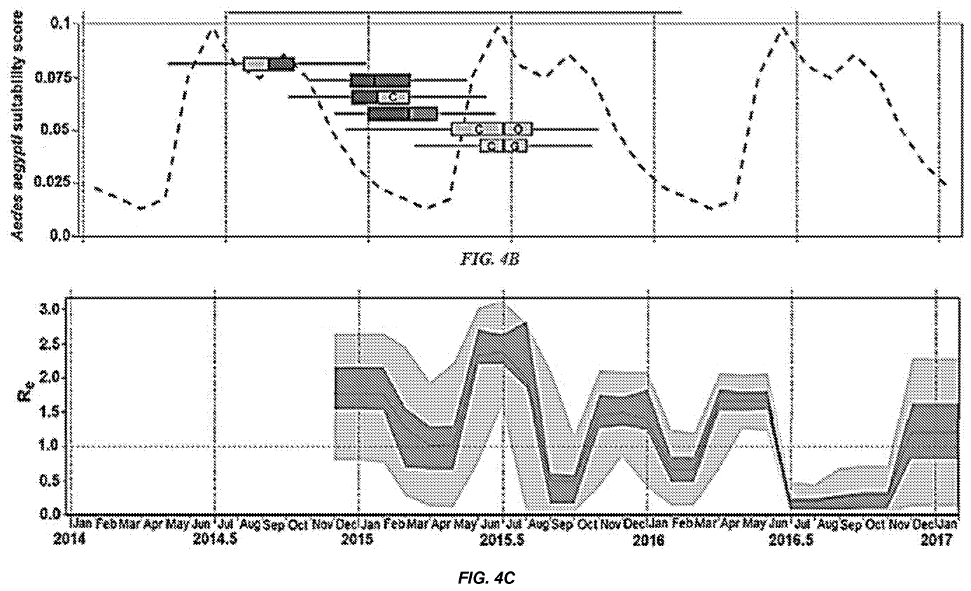

A regression of genetic divergence against sampling time confirmed that the data set was suitable for molecular clock analysis (FIG. 3B; R²=0.65). To reconstruct the dissemination of ZIKV within Central America and Mexico, a well-established Bayesian molecular clock phylogeographic approach was used. The resulting maximum clade credibility tree was largely consistent with previous studies (FIG. 4A) and with the ML phylogeny (FIG. 3B). As before, most sequences from CAM were placed in a single clade (clade B in FIG. 4A; posterior probability=1.0). The date of the most recent common ancestor (MRCA) of clade B was estimated to be December 2014 (FIG. 4A; 95% HPD=September 2014-March 2015), diverging from Brazilian strains around July 2014 (node A in FIG. 4A; 95% HPD=March 2014-November 2014; posterior probability=0.8). Hence, clade B lineage was estimated to be exported from Brazil to Central America between July and December 2014. This timescale was approximately three months earlier than in previous studies, a refinement likely due to the larger number of strains from CAM included in the current analysis. Four ZIKV strains from Panama and Mexico did not result from the clade B introduction and were instead likely introduced from Colombia or the Caribbean during the second half of 2015 (clade C; FIG. 4A).

A discrete trait analysis was used to infer the ancestral location of each phylogeny branch. This indicated that the most likely location of the common ancestor of clade B was Honduras (FIG. 4A; posterior probability=0.97). This result was unlikely to be an artefact of sampling intensity because clade B contained more sequences from Mexico (n=47) than from Honduras (n=31) and because randomly subsampling of the dataset confirmed that this was the most likely scenario (FIG. 7). Despite being smaller and less populous than Mexico, Honduras accounted for >50% of all suspected ZIKV cases in the CAM region (WHO 2017) and exhibited the highest average environmental suitability for ZIKV vectors (FIG. 3). The phylogeographic analysis estimated that ZIKV was introduced to Honduras from Brazil around July-September 2014 (FIG. 4B), coinciding with high environmental suitability for Aedes aegypti mosquitoes across Honduras (FIG. 4B). It was found that subsequent dissemination of ZIKV to Guatemala and Nicaragua and to Southern Mexico likely occurred during early 2015, when vector suitability in Honduras was declining (FIG. 4B). The state-level sampling of viruses from Mexico indicated that ZIKV was most likely first introduced into Mexico (from Honduras) via the southern state of Chiapas. The reconstruction suggested that ZIKV subsequently spread within Mexico, from Chiapas to Oaxaca and Guerrero states, and that this within-country movement occurred in mid-2015 (FIG. 4B).

The Bayesian birth-death skyline model was used to estimate temporal changes in Rₑ, the effective reproductive number of the CAM clade of ZIKV, directly from sequence data (FIG. 4C). For each point in time, Rₑ represented the average number of secondary infections caused by a case (hence Rₑ>1 and Rₑ<1 represented epidemic growth and decline, respectively). Four periods of epidemic growth (estimated Rₑ>1; red dotted line in FIG. 4C) were observed within 2015 and 2016, although only the second and fourth periods were statistically significant (i.e., posterior probability that Rₑ>1 is >=95%). The first period coincided with ZIKV spread from Honduras to other CAM countries. The second growth period, during mid-2015, reached a median Rₑ>2 and coincided with the predicted annual peak of mosquito suitability in Honduras (FIG. 4C). This second period corresponded to a rapid radiation of ZIKV lineages in clades B and C (FIG. 4A) and preceded the first reported cases of ZIKV in Central America and Mexico. The third growth period occurred immediately prior to the rapid increase in reported ZIKA cases in Honduras in early 2016, when the predicted vector suitability was low (FIG. 3). The fourth period corresponded to the epidemic observed during April-July 2016 in all countries except El Salvador and Panama (FIG. 3).

In the study, the genetic diversity and transmission history of ZIKV in Central America and Mexico was discovered, and a “spiked primer” enrichment strategy was developed for low-titer viral genome recovery from clinical samples. 61 complete and partial ZIKV genome sequences were reported, representing infections from returning travelers to the USA and autochthonous infections of residents of Mexico, Nicaragua, Honduras, Guatemala, and El Salvador. Using a combination of phylogenetic, epidemiological, and environmental data, the introduction and spread of ZIKV in Central America and Mexico were revealed. The “spiked primer” enrichment strategy was also demonstrated was suitable for elucidated the capacity for metagenomic detection of pathogenic microorganisms other than ZIKV, as well as co-infections, and may thus constitute a generalizable approach for rapid genomic surveillance of future outbreaks. The enrichment strategy demonstrated herein for pathogen detection could also be used to facilitate whole-genome sequencing of pathogens, antiviral resistance, virulence characterization, and pathogen discovery.

Robust viral genome sequence recovery from low-titer clinical samples is a substantial technical challenge for viral genomic epidemiology. ZIKV is difficult to sequence given the brief period of detectable viremia and relatively lower viral titers in returning travelers, for whom medical care is often delayed. Here, short 13-nt spiked primers and/or oligonucleotide capture probes were used to sufficiently enrich low-titer clinical samples for genome recovery by metagenomic next-generation sequencing. Short 13-nt primers used for reverse transcription have the advantage of being less affected by self- and cross-dimerization, which can lead to preferential PCR amplification that often hinders multiplexed PCR designs. Moreover, while typical multiplex PCR requires co-operative primer pairs with well-matched hybridization properties, with short primers, cDNA strands are primed at lower temperatures and thus should be more tolerant of primer-target mismatches. Oligonucleotides used for bait capture are even more lenient because they require no exact match at their 3′ terminus. Consequently, spiked primer and probe-based enrichment approaches can detect and sequence diverse strains of a target virus, whereas multiplexed PCR is generally lineage-specific.

A metagenomic approach for infectious disease diagnosis and surveillance, such as the spiked primer strategy reported here, could be particularly useful in outbreak settings. Accurate identification of the causative pathogen is important for targeted treatment and containment to prevent transmission. A metagenomic approach that can identify co-infections and other infectious agents is particularly useful if pathogenic microorganisms co-circulate in the outbreak region. For example, nearly 25% of suspected Ebola virus patients in Liberia in 2014-15 were found to be infected with Plasmodium falciparum malaria instead.

Spiked primers for target enrichment as demonstrated herein, did not increase overall sample turnaround time for metagenomic sequencing, unlike bait capture oligonucleotide probes, which are more costly and incur additional hybridization times ranging from 6-24 hours. Finally, spiked primer sets can be pooled for differential diagnosis of febrile illness from pathogenic microorganisms co-circulating within a given area (e.g., an “arboviral” panel consisting of ZIKV, DENV, CHIKV, and West Nile Virus) or to broadly capture viral diversity (e.g., sequencing of diverse HIV-1 strains in Africa).

A perennially warm and humid climate makes many locations in Central America and Mexico (CAM) susceptible to mosquito-borne diseases. The first ZIKV cases in CAM were reported in November 2015, about one year earlier than the inferred date of introduction of ZIKV in the region (FIGS. 4A and B). The phylogenetic analyses show that ZIKV was introduced into CAM multiple times, but only one such introduction has become epidemiologically dominant and spread between countries in the region. This lineage (clade B; FIG. 4A) was inferred as originating from Brazil, where ZIKV transmission is thought to have been established since early 2014. The introduction into Honduras likely occurred in mid-2014, when the country had a notably high predicted environmental suitability for ZIKV vectors (FIG. 3 and FIG. 4B). Thus, ZIKV circulated in CAM for at least a year before being first detected there in November 2015, corroborating previous reports of undetected ZIKV spread in this and other regions in the Americas. The analysis suggests that ZIKV spread from Honduras to other countries in Central America and Mexico between late 2014 and early 2015, when predicted mosquito suitability in Honduras was declining (FIG. 4B). Alternatively, if current data underestimates the genetic diversity of ZIKV in Honduras, then dissemination events from Honduras may have been more numerous and some could be more recent (i.e., during mid-015), when predicted vector suitability was higher. Both scenarios are consistent with the reported presence of ZIKV in Southeastern states of Mexico in January-March 2015.

Time lags between the estimated date of ZIKV introduction into a location, and its date of first detection, have been reported across the Americas, including in regions with established surveillance systems. Early detection of ZIKV was rendered difficult by a large number of asymptomatic patients, and the similarity between ZIKV-associated symptoms and those of other arboviruses, particularly DENV and CHIKV. New surveillance methods, such as large-scale active mosquito trapping, could provide timely information that could inform epidemic response and control interventions.

Multiple lines of evidence point to complex annual trends in ZIKV transmission in CAM, contrasting with a single transmission season observed in other locations. Firstly, reported suspected ZIKV cases in 2016 and 2017 peak twice a year in Belize, Honduras and Guatemala (FIG. 3) and a 2016 winter epidemic was reported in El Salvador. Winter transmission is notable because, at that time, predicted climatic suitability for ZIKV vectors was low (FIG. 3). Secondly, the time series of qRT-PCR positive samples exhibits two waves within 2016; a larger wave in spring and summer dominated by samples from Mexico and Nicaragua, and a smaller winter wave comprising samples from the Atlantic island of Roatán in Honduras, Guatemala, El Salvador, Nicaragua and elsewhere (FIGS. 1A and 1B). Thirdly, genetic estimates of the ZIKV clade B effective reproductive number (Rₑ) (FIG. 4C) reveal periods of epidemic growth approximately every six months. Lastly, ZIKV lineage movements among countries also occur in both winter and summer (FIG. 4B). Although each of these observations carries substantial uncertainty, their convergence is striking, and there is evidence for sustained transmission of CHIKV in Honduras in January-March 2015, in addition to a CHIKV epidemic there later in the year.

The reasons for these epidemiological trends remain unclear and a number of hypotheses can be put forward: (1) it is possible that ZIKV cases were over-reported in some locations in late-2015, perhaps due to heightened awareness immediately following the first suggestions of a link between ZIKV and microcephaly; (2) ZIKV introduction into a wholly susceptible population might generate substantial transmission even when vector abundance is comparatively low. If true, this implies that little herd immunity accumulated in the CAM region during 2014 and 2015; (3) trends in predicted vector suitability at the national level may hide strong local environmental heterogeneity; for example, between highlands and lowlands, and between the Pacific and Atlantic coasts. Brooks et al., (2017) reported a February 2016 peak in ZIKV cases on Roatán island, Honduran Bay, whose climate differs from the Honduran mainland. Some locations may also be capable of sustaining year-round transmission; (4) the contribution of latent infection and sexual transmission to ZIKV incidence is currently not well characterized, and requires further investigation. However, sexual transmission is unlikely to cause a large proportion of reported cases (see, FIG. 3).

### Example 2

Clinical ZIKV serum samples from Mexico were collected as part of the national epidemiological surveillance program of Instituto Mexicano del Seguro Social (IMSS), a branch of the Ministry of Health, as previously described. Samples along with ancillary clinical and epidemiological data were de-identified prior to analysis, and are thus considered exempt from human subject regulations with waiver of informed consent according to 45 CFR 46.101(b) of the United States Department of Health and Human Services. Analysis of whole blood samples from patients with Ebola virus disease was approved by the Ministry of Health in the Democratic Republic of the Congo. Patients in the 2014 Boende EBOV outbreak from Aug. 13, 2014 to Sep. 8, 2014 and in the 2018 North Kivu EBOV province outbreak (Aug. 1, 2018 to present) provided oral consent for study enrollment and collection and analysis of their blood. Consent was obtained at the homes of patients or in hospital isolation wards by a team that included staff members of the Ministry of Health. Plasma samples from patients with HIV-1 and/or Usutu virus infection were provided by the Abbott Global HIV-1 Surveillance Program. Briefly, informed consent was obtained for collection of HIV-1 infected blood donations from blood banks in Cameroon and analysis for viral load determination and sequencing under protocols approved by local ethics committees. Clinical samples were analyzed at University of California, San Francisco (UCSF) under protocols approved by the UCSF Institutional Review Board (protocol #11-05519).

Clinical sample collection. Viral cultures of ZIKV (Uganda strain), DENY (type 1), and MS2 bacteriophage were purchased from American Type Culture Collection (ATCC, Manassas Va., USA). Ebola cultures Kikwit strain in TRIzol LS (Thermo Fisher Scientific, Waltham, Mass., USA) was provided by Dr. Jean Patterson at Texas Biomedical Research Institute (San Antonio, Tex.). Clinical ZIKV serum samples were collected by Central Laboratory of Epidemiology (CLE), IMSS in Mexico City, Mexico. Real-timez quantitative reverse transcription PCR (RT-PCR) testing was used for ZIKV detection and viral titer determination by standard curve analysis. Forward and reverse primers (ZIKV 1086 and ZIKV 1162c, respectively) and Carboxyfluorescein (FAM)-labelled probes (ZIKV 1107-FAM) were used as previously described. Clinical Ebola samples collected from patients in the 2014 Boende and 2018 North Kivu province outbreaks were provided by Dr. Placide Mbala and colleagues at INRB in Kinshasa, DRC. Clinical HIV and hepatitis C plasma samples were obtained from the UCSF Clinical Microbiology Laboratory (San Francisco, USA). The CSF sample from a patient with POWV meningoencephalitis was provided by Boston Children's Hospital. The CSF sample from a patient from SLEV meningoencephalitis was provided by University of California, Los Angeles (UCLA) Medical Center. Negative plasma sample matrix used as a “no template” control (NTC) was obtained from Golden West Biologicals Inc. (Temecula, Calif., USA).

MSSPE viral spiked primer design. Multiple sequence alignment (MSA) of viral genomes (downloaded from NCBI GenBank as of September 2017) was performed using MAFFT at default parameters (algorithm=“Auto”, scoring matrix=“200PAM/k=2”, gap open penalty=1.53, offset value=0.123). An in-house bioinformatics pipeline named “MSSPE-design” was developed on an Ubuntu Linux computational server for automated design of spiked primers. Briefly, the MSA-aligned genomes were partitioned into overlapping 500 nucleotide (nt) segments with 250 nt overlap using PYFASTA ([http://] [pypi.python.org/pypi/pyfasta/]). Forward or reverse 13 nt primers were selected from 50 nt regions at the ends of each segment by iteratively ranking candidate 13mer (“k-mer”) sequences in reverse order by frequency, selecting the top kmer shared by the most segments and not containing any ambiguous nucleotides, and then removing segments sharing that 13mer before repeating the process on the remaining segments. To decrease overall spiked primer costs, the iterations were repeated until the number of remaining segments containing a shared kmer was below a pre-designated threshold (ranging from n=1 for viruses with only a limited number of genomes/genome segments such as CCHF to n=10 for viruses comprising thousands of genomes and multiple genotypes such as DENV). Spiked primers were filtered by removal of primers with melting temperatures (Tm) greater than 2 standard deviations from the mean or that were predicted to self-dimerize or cross-dimerize with a ΔG value of −9 kcals/mol or more negative.

Spiked primers were ordered and synthesized by Integrated DNA Technologies Inc. (IDT, Coralville, Iowa, USA). Forward or reverse spiked primer oligonucleotides targeting individual viruses were synthesized on a 10 nmole scale in 96-well plates with standard desalting and 6 nm of each individual oligonucleotide were mixed and then resuspended to a final volume of 500 μL in IDTE pH 8.0. Spiked primer panels (ArboV, HFV, and A11V) were designed by mixing the spiked primers for each individual virus in equimolar ratios and then diluting with TE (Tris-EDTA) buffer to the desired concentration. The estimated cost per reaction for individual virus-specific primers was $0.06-$0.08, and average cost per reaction for spiked primer panels was $0.17-$0.34.

Construction of metagenomic sequencing libraries. Viral RNA was extracted from 200 μL of contrived or clinical patient samples using the EZ1 Advanced XL BioRobot and EZ1 Virus Mini Kit (Qiagen, Redwood City, Calif.), with the exception of EBOV RNA, which was extracted manually in the viral hemorrhagic fever reference laboratory in INRB, Kinshasa using the Direct-zol RNA MiniPrep Kit (Zymo Research, Irvine, Calif.). 25 μL of nucleic acid extract was treated with DNase (3 μL Turbo DNase, 1 μL Baseline, 5 μL Turbo buffer and 16 μL nuclease-free water), and incubated on an Eppendorf ThermoMixer at 37° C., 600 rpm for 30 min. The Zymo RNA Clean and Concentrator kit (Zymo Research, Irvine, Calif.) was used to clean up DNase-treated RNA, and the final RNA was eluted in 32 μL water. The RNA was then mixed with random hexamer (RH) alone (1 μM) or spiked primer plus RH in a 10:1 ratio of spiked primer to RH, and heated to 65° C. for 5 min. The reverse transcription master mix (10 μL SuperScript III buffer, 5 μL dNTP of 12.5 mM, 2.5 μL DTT of 0.1M, 1 μL SuperScript III enzyme) was added to each sample and incubated at 25° C. for 5 min, followed by 42° C. for 30 min and 94° C. for 2 min. After cooling to 10° C., a second-strand synthesis master mix (3.7 μL Sequenase buffer, 0.225 μL Sequenase enzyme and 1.1 μL water) was added to each reaction, followed by a slow 2 min ramp to 37° C. and 8 min incubation. The resulting cDNA was cleaned up using the Zymo DNA Clean and Concentrator kit (Zymo Research, Irvine, Calif.), with the addition of 10 μL linear acrylamide to each sample, and eluted in 10 μL water. Using the Illumina Nextera XT kit, 2.5 μL sample cDNA was incubated at 55° C. for 5 mins in tagmentation mix (10 μL TD buffer and 5 μL ATM enzyme), and immediately neutralized with 2.5 μL NT buffer. 12.5 μL of tagmented DNA was then transferred to reaction tube containing indexing mix (7.5 μL Nextera XT NPM, 2.5 μL N-7xx primer and 2.5 μL S-5xx primer), followed by PCR amplification (72° C. for 3 min, 95° C. for 30 s, followed by 16 cycles of denaturation (95° C. for 10 s), annealing (55° C. for 30 s), and extension (72° C. for 30 s), with a final extension at 72° C. for 5 min). After PCR, 3 μL of PCR product was analyzed by 2% gel electrophoresis to check for library size and band intensity. If no band or only a very faint band was observed on the gel, another round of recovery PCR was performed. For recovery PCR, the library was washed using 0.9× AMPure XT beads (Beckman Coulter, Carlsbad, Calif., USA) and 5 μL clean library was mixed with 45 μL master mix (10 μL buffer, 2.5 μL of 10 uM Nextera general primers, 1 μL dNTP, 0.5 μL Phusion DNA polymerase enzyme and 31 μL water), followed by a 95° C. incubation for 30 s and 10 cycles of PCR (95° C. for 30 s denaturation, 60° C. for 30 s annealing, and 72° C. for 30 s extension), with a final extension at 72° C. for 5 min. The final cDNA library was eluted in 20 μL EB buffer after a wash step using 0.9× AMPure beads.

Metagenomic sequencing. The cDNA libraries were quantified using the Qubit fluorometer (Thermo Fisher Scientific) and the sizes of the libraries were measured using Agilent Bioanalyzer (Agilent Technologies, Santa Clara, Calif.). Illumina sequencing was performed on a MiSeq instrument using 150 nt single-end runs according to the manufacturer's protocol. For nanopore, amplified cDNA libraries from Nextera library preparation were end-repaired and ligated with adapter and motor proteins using the 1D Ligation Sequencing Kit (Oxford Nanopore Technologies). Metagenomic libraries for nanopore sequencing were run on R9.4 or R9.5 flow cells, using either a MinION MK1B or GridION X5 instrument (Oxford Nanopore Technologies).

Capture probe enrichment for ZIKV samples. The xGen Lockdown Kit (IDT Technologies, Redwood City, Calif.) was used for capture probe enrichment of ZIKV. Briefly, barcoded amplified cDNA libraries corresponding to each sample were mixed in equimolar proportions to generate a 500 ng pooled library. The pooled library was then added to a hybridization mix containing ZIKV xGen Lockdown probes, and the hybridization reaction was performed by incubation at 65° C. for 16 h, followed by streptavidin bead capture for 45 min. Beads containing captured cDNA were re-suspended in an amplification reaction mix (25 μL KAPA HiFi HotStart ReadyMix, 1.25 μL xGen primer and 3.75 μL water), and post-capture PCR was performed (98° C. for 45 s, followed by 10 cycles of denaturing (98° C. for 15 s), annealing (60° C. for 30 s), and extension (72° C. for 30 s), with a final extension at 72° C. for 1 min). PCR amplicons were purified using 1.5× volume of AMPure XP beads and finally eluted in 20 μL EB buffer. Purified PCR products were analyzed by 2% gel electrophoresis to check library size, and DNA concentration was estimated using the Qubit fluorometer. The capture probe enriched library was run on an Illumina MiSeq instrument using 150 nt single-end runs according to the manufacturer's protocol.

Tiling multiplex PCR enrichment for ZIKV. Tiling multiplex PCR for ZIKV enrichment was performed according to the “Primal” protocol described by Quick et al., except for libraries prepared using both MSSPE and tiling multiplex PCR, for which an AMPure bead wash of 1.2× was performed immediately after cDNA synthesis (before adding multiplexed primers) to remove residual ZIKV MSSPE primers (4 μM) that had been added during the reverse transcription step. After visualization of a PCR band of the expected size (400 nt) by 2% gel electrophoresis, barcoded sequencing libraries were prepared using the NEBNext Ultra II DNA Library Preparation Kit (New England BioLabs, Inc., Ipswich, Mass.), and sequenced on an Illumina MiSeq instrument using 250 nt paired-end runs according to the manufacturer's protocol.

Bioinformatics pipelines for viral detection and reference genome alignment. Sequencing data from Illumina MiSeq or HiSeq instruments were analyzed for viruses using the SURPI+ (“sequence based ultra-rapid pathogen identification”) computational pipeline (UCSF), a modified version of a previously published bioinformatics analysis pipeline for pathogen identification from mNGS sequence data. Specifically, the SURPI+ pipeline modifications include (i) updated reference databases based on the NCBI nt database (March 2015 build), (ii) a filtering algorithm for exclusion of false-positive hits from database misannotations, and (iii) taxonomic classification for species-level identification. Viral reads were mapped to reference genome and percent coverage determined an in-house developed SURPIviz graphical visualization interface or Geneious software v10. For virus detection from nanopore reads, an in-house developed pipeline called SURPIrt (SURPI “real-time”, unpublished) was used, which identifies viral reads by Bowtie2 alignment to the NCBI Viral RefSeq database or the viral portion of the NCBI nt database. Viral reads obtained by nanopore sequencing were mapped to reference genomes using GraphMap.

Quantification and Statistical Analysis. CAL ANALYSIS. The RPM (reads per million) metric was calculated as the number of viral species-specific reads divided by the number of preprocessed reads (reads remaining after adapter trimming, low-quality filtering, and low-complexity filtering of raw reads) for Illumina sequencing, or the number of viral species-specific reads divided by the number of basecalled reads for nanopore sequencing. The fold change for MSSPE enrichment was defined as the RPM obtained for a target virus using MSSPE divided by the RPM obtained using RH priming only. The median fold change is given instead of the mean fold change if the data contained outliers. The percent increase in genome coverage is the genome coverage obtained using RH alone subtracted from that obtained using MSSPE. Chi-squared test was used to compare two proportions, and p value less than 0.05 is considered statistically significant.

Spiked primer design. A general method for viral enrichment and genome recovery from clinical samples for use in diagnostics, public health surveillance, and outbreak investigation was developed. The method was developed to (i) be applicable for any targeted virus, regardless of its degree of representation in reference databases (e.g., from 60 to 3,571 reference genomes/genome segments) (FIG. 10A), (ii) preserve broad metagenomic sensitivity for comprehensive detection of known and novel pathogenic microorganisms (viral and non-viral) and co-infections, (iii) not affect overall turnaround times for sample processing, and (iv) enrich mNGS libraries sufficiently to allow robust viral genome recovery from low-titer clinical samples. Specifically, an automated computational algorithm was designed which took as input an arbitrary set of reference genomes and constructed a minimal panel of short, 13-nt spiked primers covering these genomes (FIG. 10A), to be added during the cDNA synthesis (reverse transcription step of mNGS library preparation (FIG. 10B). Spiked primers were designed for 14 viruses, in total comprising 6,102 primers and including vector-borne and/or hemorrhagic fever viruses of public health significance.

MSSPE for viral pathogen detection. First, the enrichment effect of virus-specific spiked primers for ZIKV and West was evaluated. Nile virus (WNV) detection using mNGS on a benchtop sequencing platform (Illumina MiSeq). At a spiked primer concentration of 1 μM, the maximum concentration generally recommended for specific PCR (Lorenz, 2012), the degree of ZIKV enrichment in contrived samples containing ZIKV and either HIV or hepatitis C virus (HCV) as an off-target virus was highest (5-6×) at 5:1 and 10:1 molar ratios of spiked to random hexamer (RH) primers. There was no or minimal loss of detection sensitivity for off-target HIV and HCV; rather, HIV reads were enriched (2.7×) in the presence of ZIKV spiked primers. Increasing the molar ratio of spiked to RH primers to 100:1 from 10:1 did not result in increased enrichment of WNV reads using the arbovirus spiked primer panel (ArboV) at 1 μM concentration (SEQ ID NOs:399-1562). A comparison of spiked primer concentrations of 1 μM, 4 μM, and 10 μM at molar ratios of 10:1 found that the degree of enrichment peaked at 4 μM.

Next, spiked primer concentrations ranging from 1 μM to 40 μM or 80 μM for enrichment of ZIKV, DENV, EBOV, and off-target MS2 bacteriophage (an RNA virus) using spiked primer panels [arboviruses (ArboV), hemorrhagic fever viruses (HFV), and all viruses (Ally, all viruses with the exception of HCV], aiming to determine the optimal concentration for the panels was tested. The peak performance of the ArboV panel was found at a primer concentration of 10 or 20 μM, yielding an ˜11× enrichment in ZIKV and −5× enrichment in DENV reads (FIG. 11A). Metagenomic detection of off-target viruses (EBOV and MS2 phage) was not impaired; in fact, low-level enrichment was observed (FIG. 11A). The optimal primer concentration for the HFV panel was found to be 20 μM (FIG. 11B), yielding a mean 5× enrichment for EBOV. The A11V panel at the optimal 10 μM primer concentration yielded 3-28× enrichment of ZIKV, DENV, and EBOV reads (FIG. 11C).

As the degree of enrichment was noted to be higher at lower viral titers (FIGS. 11A and C), virus-specific primers and expanded panels for enrichment of ZIKV, dengue virus (DENV), and EBOV across a 3-log dilution of viral titers (10, 100, and 1,000 copies/mL) were tested. At the previously determined optimal concentration 4 μM and molar ratio of 10:1 spiked to RH primers, enrichment of individual viruses across the 3 concentrations using virus-specific primers ranged from 3×-55×. Across all primer sets, the highest degree of enrichment overall was observed at the lowest titer (˜10-40× at 10 copies/mL), with less enrichment (˜4-15×) at titers of 100 or 1,000 copies/mL (FIGS. 11D-G). Enrichment of EBOV using the HFV panel averaged 11× and enrichment of ZIKV and DENV using the ArboV panel averaged 9× and 5×, respectively (FIG. 11H). Using all 4,792 primers in combination (A11V) yielded ˜12× increases in the number of viral reads for each of the 3 targeted viruses (FIG. 11H).

The performance of the spiked primer panels was then evaluated on the MinION portable nanopore sequencing platform (Oxford Nanopore Technologies, Oxford, UK) (Table 1). Overall levels of ZIKV, EBOV, and DENV enrichment at viral titers ranging from 10-1,000 copies/mL were comparable for the two platforms (median enrichment of 7.8× on the MinION and 9.2× on the Illumina MiSeq). The use of spiked primer panels enabled detection of ZIKV and EBOV down to 10 copies/mL, near the limits of detection for virus-specific PCR, whereas no ZIKV or EBOV reads were obtained by mNGS using RH primers alone (Table 1).

TABLE 1
Detection of targeted viruses using MSSPE

| | | | Viral | | Fold | | |
|---|---|---|---|---|---|---|---|
| | | | RPM | | change | Viral | Fold |
| | | | (ONT | | (ONT | RPM | Change |
| | Viral titer | | MinION | | MinION | (Illumina | (Illumina |
| Virus | (copies/ml) | Primer type | nanopore) | # of reads | nanopore) | MiSeq) | MiSeq) |
| ZIKV | 10 | RH | 0 | 141,549 | | 0 | |
| ZIKV | 10 | ArboV-SP | 8 | 378,382 | >8.0 | 3.1 | >3.1 |
| ZIKV | 10 | RH | 0 | 131,662 | | 0.68 | |
| ZIKV | 10 | ArboV-SP | 28 | 393,179 | >28 | 11.2 | 16.4 |
| EBOV | 10 | RH | 1 | 810,439 | | 0.27 | |
| EBOV | 10 | EBOV-SP | 14 | 1,140,463 | 14 | 15 | 55 |
| EBOV | 10 | HFV-SP | 2 | 489,000 | 2.0 | 2 | 7.4 |
| EBOV | 100 | RH | 0 | 645,349 | | 8 | |
| EBOV | 100 | EBOV-SP | 108 | 608,429 | >108 | 130 | 16 |
| EBOV | 100 | HFV-SP | 31 | 386,053 | >31 | 53.6 | 6.7 |
| ZIKV | 100 | RH | 31 | 252,145 | | 16 | |
| ZIKV | 100 | ZIKV-SP | 341 | 259,154 | 11 | 247 | 15 |
| DENV | 100 | RH | 42 | 307,846 | | 38 | |
| DENV | 100 | DENV-SP | 62 | 763,017 | 1.5 | 199 | 5.2 |
| DENV | 100 | ArboV-SP | 103 | 202,737 | 2.5 | 141 | 3.7 |
| EBOV | 1,000 | RH | 216 | 244,554 | | 358 | |
| EBOV | 1,000 | EBOV-SP | 6,117 | 472,770 | 28 | 7,563 | 21 |
| ZIKV | 1,000 | RH | 208 | 125,000 | | 66 | |
| ZIKV | 1,000 | ZIKV-SP | 1,570 | 70,000 | 7.5 | 1,238 | 19 |
| ZIKV | 1,000 | ArboV-SP | 1,325 | 81,499 | 6.3 | 740.7 | 11 |
| DENV | 1,000 | RH | 322 | 433,949 | | 240 | |
| DENV | 1,000 | DENV-SP | 1,431 | 338,789 | 4.4 | 1,511 | 6.3 |
| DENV | 1,000 | ArboV-SP | 699 | 520,593 | 2.2 | 945 | 4.0 |

The performance of the ArboV and HFV panels using clinical blood samples from ZIKV (n=5) and EBOV-infected (n=5) patients from Mexico and DRC (2014 Boende outbreak), respectively. A median viral enrichment of 2.1× was observed, resulting in 10 of 10 (100%) of samples being detected using the Illumina MiSeq platform, versus only 8 of 10 (80%) by mNGS with RH primers alone. Analysis of a subset of samples (n=3) by nanopore sequencing revealed similar levels of enrichment to those obtained on the Illumina MiSeq platform that, in one case, enabled detection of ZIKV in a low-titer clinical sample negative by randomly primed mNGS.

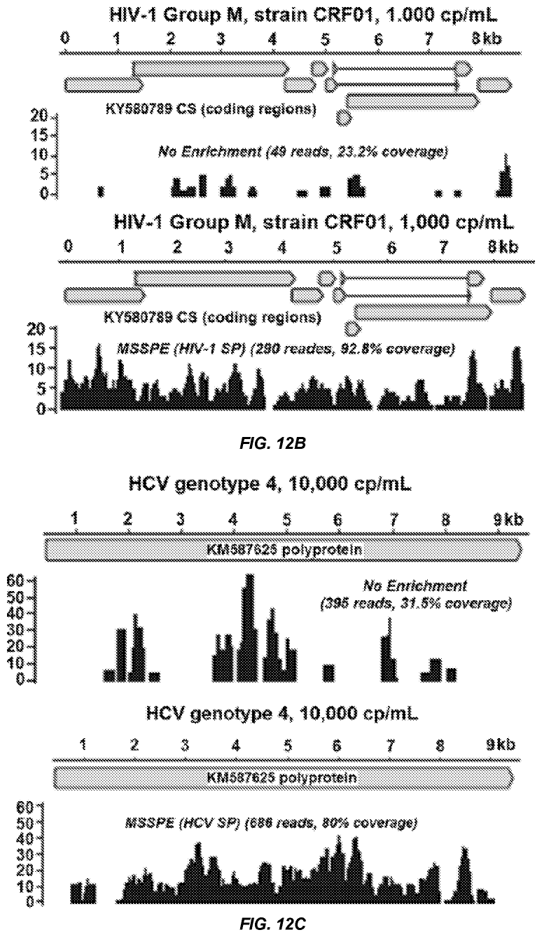

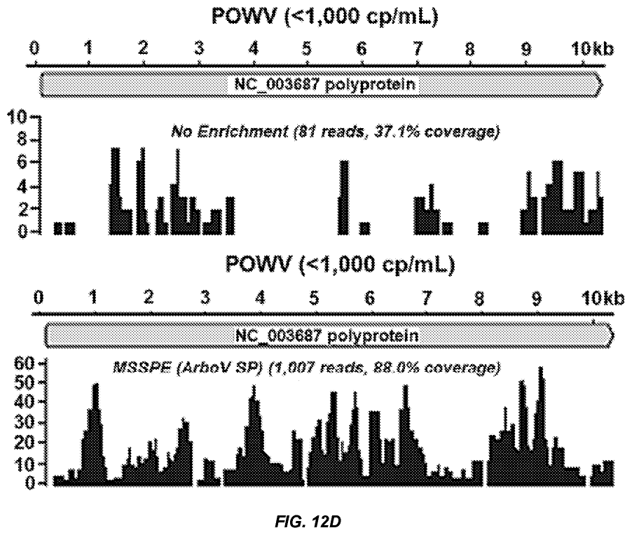

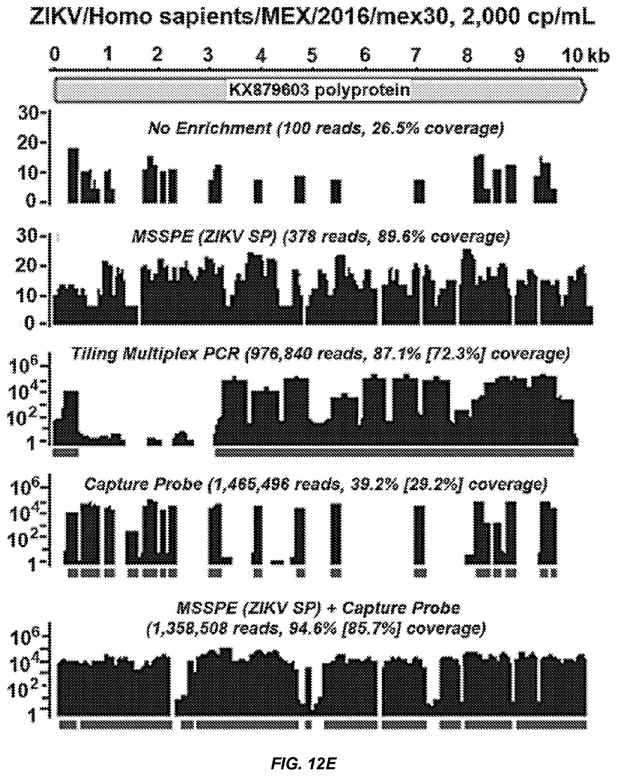

MSSPE for virus genome sequencing. It was hypothesized that the increased proportion of viral reads obtained using the MSSPE method would improve genome coverage. Using ZIKV spiked primers on plasma samples spiked with 1,000 copies/mL of ZIKV more than doubled the genome coverage obtained using RH primers only, from 35.8% to 72.8%. The performance of virus-specific primers for genome sequencing of ZIKV, DENV, EBOV, HIV-1 (divergent and recombinant strains from Cameroon and DRC, Africa), and HCV (genotypes 2, 4, and 6 from California, United States) were then evaluated. On average, a 49% (±13.9% SD) increase in genome coverage was achieved using spiked primer relative to RH primers only for contrived ZIKV, DENV, HIV and EBOV samples at titers of 100-1,000 copies/mL (Table 2, FIG. 12A), and a 42% (±15.0% SD) increase in genome coverage for clinical HIV-1 and HCV samples at titers ranging from 100-10,000 copies/mL (Table 2, FIGS. 12B and C). Similarly, a 36.5% (±16.8% SD) increase in genome coverage was obtained using spiked primer panels (ArboV, HFV, and AllV) for contrived and clinical samples of ZIKV, DENV, and EBOV. No significant gains in genome coverage were observed at a titer of 10 copies/mL, a finding attributed to insufficient sequencing depth. In addition, the MSSPE method was tested using EBOV and DENV spiked for genome recovery on the MinION nanopore sequencer. With contrived samples at a titer of 1,000 copies/mL, comparable percentage increases in genome coverage were achieved on both ONT MinION nanopore and Illumina MiSeq sequencing platforms (Table 2).

MSSPE for pathogen discovery. To assess the utility of MSSPE for pathogen discovery, spiked primers were tested which could enrich for sequences from emerging flaviviruses in clinical samples from infected patients. Of note, flaviviruses had not been specifically targeted in the initial spiked primer design. ZIKV spiked primers were used to enrich for St. Louis encephalitis (SLEV), whereas ArboV panel spiked primers were used to enrich for Powassan virus (POWV) in patient cerebrospinal fluid (CSF) samples. The use of ZIKV spiked primers enriched the number of reads to SLEV by ˜3×, with a corresponding increase in 17.5% genome coverage (Table 3). In CSF from a patient with tick-borne POWV meningoencephalitis, the use of ArboV spiked primers enriched for POWV reads by 15× over RH primers alone, and improved viral genome coverage by 43% (Table 3 and FIG. 12D).

TABLE 2
Improved viral genome coverage using MSSPE

| | | | | | | Genome | | Genome | |
|---|---|---|---|---|---|---|---|---|---|
| | | | | | # of viral | coverage | # of viral | coverage | |
| | | Viral titer | | Fold | reads (RH | (RH | reads (spiked | (spiked | % increase in |
| Virus | Strain/subtype | (copies/ml) | Primer type^{a} | enrichment | primers) | primers) | primers) | primers) | coverage |
| ZIKV | Uganda MR766 | 100 | ZIKV-SP | 10.0 | 31 | 23.70% | 309 | 67.2% | 43.5% |
| ZIKV | Uganda MR766 | 100 | ZIKV-SP | 15.4 | 13 | 4.30% | 200 | 44.3% | 40.0% |
| ZIKV | Uganda MR766 | 1,000 | ZIKV-SP | 19.2 | 64 | 46.20% | 1229 | 95.6% | 49.4% |
| HIV-1 | Group M, CRF01 | 100 | HIV-SP | 10.5 | 17 | 12.30% | 179 | 66.4% | 54.1% |
| DENV | type 1 | 100 | DENV-SP | 5.2 | 69 | 29% | 359 | 80.9% | 51.9% |
| DENV | type 1 | 1,000 | DENV-SP | 6.3 | 382 | 67.50% | 2411 | 97.2% | 30.0% |
| EBOV | Ebola Kikwit-95 | 100 | EBOV-SP | 10.5 | 200 | 7.50% | 2095 | 84.9% | 77.4% |
| EBOV | Ebola Kikwit-95 | 1,000 | EBOV-SP | 21.0 | 385 | 45.20% | 8095 | 90.3% | 45.1% |
| Mean (SD) | | | | | | | | | 49 (±13.8%) |
| HIV | CRF01, #8 | 1,000 | HIV-SP | 6.0 | 55 | 21% | 330 | 92% | 71.0% |
| HIV | CRF01, #9 | 1,000 | HIV-SP | 3.1 | 76 | 44.80% | 234 | 83% | 38.2% |
| HIV | CRF01, | 10,000 | HIV-SP | 2.6 | 138 | 67% | 358 | 88% | 21.0% |
| | #18 | | | | | | | | |
| HIV | URF-0201, | 1,000 | HIV-SP | 2.3 | 74 | 47.80% | 167 | 74.6% | 26.8% |
| | #22 | | | | | | | | |
| HIV | URF-0122, | 1,000 | HIV-SP | 8.8 | 11 | 13.50% | 97 | 61.4% | 47.9% |
| | #20 | | | | | | | | |
| HCV | Genotype 2 | 1,000 | HCV-SP | 5.0 | 14 | 11.40% | 70 | 54% | 42.6% |
| HCV | Genotype 4 | 10,000 | HCV-SP | 1.7 | 411 | 33.30% | 707 | 82.8% | 49.5% |
| HCV | Genotype 6 | 1,000 | HCV-SP | 3.0 | 30 | 16.40% | 91 | 55% | 38.6% |

| | |
|---|---|
| Mean (SD) | 42% (±15.3%) |
| Overall mean % increase in coverage | 45.4% (±14.5%) |

TABLE 3
Detection of untargeted emerging or novel viruses using MSSPE

| | | | | | | Genome | | | Genome | % | |
|---|---|---|---|---|---|---|---|---|---|---|---|
| | | | # of | # of viral | Viral RPM | coverage | # of viral | Viral RPM | coverage | increase | |
| | Clinical | Primer | preprocessed | reads (RH | (RH | (RH | reads | (spiked | (spiked | in | Fold |
| Virus | Sample | type | reads | primers) | primers) | primers) | (spiked) | primers) | primers) | coverage | change |
| Usutu | serum | ARboV- | 122,517,964 | 114 | 0.9 | 5.5% | 845 | 6.8 | 23.0% | 17.5 | 7.5 |
| | | SP | | | | | | | | | |
| SLEV | CSF | ZIKV- | 500,000 | 96 | 192 | 67.2% | 288 | 576 | 92.8% | 25.6 | 3 |
| | | SP | | | | | | | | | |
| Powassan | CSF | ArboV- | 11,266,014 | 88 | 7.8 | 39.6% | 1,007 | 114.6 | 82.6% | 43 | 14.7 |
| | | SP | | | | | | | | | |

An HIV clinical sample was initially found to harbor Usutu virus (USUV), a flavivirus, by MSSPE using HIV-1 spiked primers. Interestingly, the degree of enrichment for USUV using these HIV-1 spiked primers over RH primers alone was 6×; subsequent analysis of the HIV-1 spiked primers found that 18 of them aligned incidentally to the USUV genome with 0 or 1 mismatches (92.3% or 100% identity). Running the same sample on the Illumina MiSeq at a limited throughput of ˜1 million raw reads resulted in detection of no USUV reads with RH primers alone, but 6 reads with the use of ArboV primers. Deeper sequencing on the Illumina HiSeq of ˜123 million reads revealed that the degree of enrichment of USUV reads using the ArboV panel was 7×(Table 3), with a corresponding increase in genome coverage of 25.6%.

Comparison of MSSPE with other target enrichment methods. A head-to-head comparison of MSSPE was performed with both capture probe and tiling multiplex PCR methods for enrichment of viral reads from ZIKV-positive clinical samples at low titers (310-28,200 copies/mL). The degree of improvement in genome coverage using MSSPE was comparable to capture probe and tiling multiplex PCR methods (Table 4). However, a small amount of cross-contamination was observed using capture probe and multiplex PCR, versus no cross-contamination using MSSPE (Negative control in Table 4). Tiling multiplex PCR for ZIKV was negative when testing a contrived ZIKV sample containing the 1947 prototype Uganda strain (Table 4), likely due to sequence divergence from the Asian lineage reference genomes from the 2014-2016 ZIKV outbreak in the Americas that were used in the initial multiplex PCR primer design.

TABLE 4
Comparison of MSSPE with other target enrichment methods

| | | Titer | RH only | | | MSSPE | | | Capture Probe^{b} |
|---|---|---|---|---|---|---|---|---|---|
| | | in | #of pre- | # of | % of | #of pre- | # of | % of | #of pre- |
| | Strain | cp/mL | processed | ZIKV | genome | processed | ZIKV | genome | processed |
| | Type | [Cₜ]^{a} | reads | reads | coverage | reads | reads | coverage | reads |
| (−) | | 0 | | | | 1,153,388 | 0 | 0 | 73,343 |
| ctrl | | | | | | | | | |
| | | [no Cₜ] | | | | | | | |
| ZIKV | Uganda | 666 | 4,923,745 | 89 | 40.2 | 5,863,273 | 485 | 91.8 | 533,673 |
| | MR766 | [40.41] | | | | | | | |
| ZIKV | mex30 | 2,020 | 1714359 | 100 | 25 | 2,343,140 | 535 | 90.8 | 2,358,242 |
| | | [38.8] | | | | | | | |
| ZIKV | Uganda | 4,650 | — | — | — | 1,673,733 | 963 | 98 | 983,752 |
| | MR766 | [37.6] | | | | | | | |
| ZIKV | mex28 | 2,670 | — | — | — | 2,673,215 | 272 | 46 | 882,771 |
| | | [38.4] | | | | | | | |
| ZIKV | mex32 | 28,200 | — | — | — | 1,924,157 | 2,541 | 100 | 5,293,168 |
| | | [35] | | | | | | | |
| ZIKV | mex | 2,490 | — | — | — | 1,714,075 | 272 | 72.7 | 1,315 |
| | 32-dil^{e} | [38.5] | | | | | | | |
| ZIKV | mex33 | 3,340 | — | — | — | 1,829,861 | 117 | 12.9 | 1,722 |
| | | [38] | | | | | | | |
| ZIKV | mex39 | 11,500 | — | — | — | 3,697,437 | 1,460 | 98.7 | 6,921,547 |
| | | [36.3] | | | | | | | |
| ZIKV | mex39- | 310 | — | — | — | 4,242,025 | 32 | 7.4 | 109,626 |
| | dil^{f} | [41] | | | | | | | |
| ZIKV | mex34 | 4,650 | — | — | — | 1,407,161 | 131 | 63.6 | 477,502 |
| | | [37.6] | | | | | | | |
| ZIKV | mex47 | 7,560 | — | — | — | 1,089,334 | 427 | 57 | 1,597,467 |
| | | [36.9] | | | | | | | |

| | | Capture Probe^{b} | | Multiplex PCR^{b} | | | |
|---|---|---|---|---|---|---|---|
| | | # of | % of | PCR | #of pre- | # of | % of |
| | Strain | ZIKV | genome | band | processed | ZIKV | genome |
| | Type | reads | coverage | on gel | reads | reads | coverage |
| (−) | | 722 | | | 1,549,271 | 77 | — |
| ctrl | | | | | | | |
| ZIKV | Uganda | 270,083 | 72.6 | (−)^{d} | — | — | — |
| | MR766 | | | | | | |
| ZIKV | mex30 | 2,015,985 | 46.8 | (+) | 2,001,198 | 1,140,475 | 83.9 |
| ZIKV | Uganda | 914,949 | 98.3 | (−)^{d} | — | — | — |
| | MR766 | | | | | | |
| ZIKV | mex28 | 763,649 | 69.9 | (−)^{d} | — | — | — |
| ZIKV | mex32 | 4,955,790 | 99 | (+) | 1,000,076 | 801,609 | 96 |
| ZIKV | mex | 141* | | (−)^{d} | — | — | — |
| | 32-dil^{e} | | | | | | |
| ZIKV | mex33 | 1,194* | | (−)^{d} | — | — | — |
| ZIKV | mex39 | 6,661,543 | 99 | (+) | 1,311,004 | 1,103,019 | 96 |
| ZIKV | mex39- | 0 | 0 | (−)^{d} | — | — | — |
| | dil^{f} | | | | | | |
| ZIKV | mex34 | 354,333 | 52.9 | (+) | 1,726,298 | 604,848 | 95.4 |
| ZIKV | mex47 | 1,395,096 | 55.4 | (+) | 3,556,785 | 971,494 | 92.2 |

^{a}ZIKV titer estimated using quantitative RT-PCR with standard curve analysis;
^{b}random hexamer primers were used for reverse transcription of RNA to cDNA prior to capture probe enrichment or tiling multiplex PCR;
^{c} plasma matrix from deidentified blood donors;
^{d}duplicate experimental replicates performed, both without a visible PCR band by gel electrophoresis;
^{e}20-fold dilution of mex32;
^{f}100-fold dilution of mex39;
Abbreviations:
Ct, cycle threshold by ZIKV quantitative RT-PCR;
RH, random hexamer;
*ZIKV reads due to cross-contamination (genome coverage depth <10 cutoff value),
— not applicable

Next, the performance of MSSPE was evaluated followed by subsequent tiling multiplex PCR or capture probe enrichment on low-titer contrived and clinical ZIKV samples (666-3,340 copies/mL). The use of spiked primers further increased the number of ZIKV reads by 3×-5× and corresponding genome coverage by 25%-80% (average 58.5±21.5%), as compared to RH primers alone (FIG. 12E). MSSPE was important for ZIKV genome recovery in the two samples tested by tiling multiplex PCR, as multiplex PCR with the standard RH priming failed to yield a distinct band on gel electrophoresis, likely due to low abundance of virus in the samples.

All patents, patent applications, and publications mentioned herein are incorporated herein by reference in their entireties for all purposes.

1. A method for designing spiked primer sequences that enrich sequencing reads for detecting a taxon or taxa of pathogenic microorganisms in a sample, the method comprising:
(i) performing multiple sequence alignments (MSA) of a plurality of genomes from a set of one or more reference genomes from a taxon or taxa of pathogens;
(ii) partitioning the MSA-aligned genomes into overlapping 300 to 600 nucleotide (nt) segments with at least a 150 nt overlap;
(iii) selecting forward and/or reverse candidate primer sequences having lengths that are within a range of 11 bp to 17 bp from 30 to 70 nt regions at the ends of each 300 to 600 nt segment by frequency of occurrence in the set of overlapping 300 to 600 nt segments:
(iv) ranking the candidate primer sequences iteratively in reverse order by frequency of occurrence in the overlapping 300 to 600 nucleotide (nt) segments;
(v) selecting top candidate primer sequences based upon the candidate primer sequences being shared by the most 300 to 600 nt segments and by not containing any ambiguous 300 to 600 nt segments;
(vi) removing 300 to 600 nt segments which share top candidate primer sequences and repeating steps (iii) to (v) until the number of the remaining 300 to 600 nt segments containing a shared candidate primer sequence is below a pre-designated threshold integer value selected from 1 to 15 in order to generate a set of top candidate primer sequences;
(viii) generating a set of spiked primer sequences from the set of top candidate primer sequences by removing top candidate primer sequences that (a) have melting temperatures (Tₘ) greater than 2 standard deviations from the mean, (b) are predicted to self-dimerize or cross dimerize with a hybridization ΔG<−9 kcal/mol or lower, and/or (c) have homopolymer repeats of greater than 5 nucleotides.

2. The method of claim 1, wherein the set of one or more reference genomes are from a taxon or taxa of pathogenic bacteria, viruses, protozoa, fungi, archaea, algae and/or eukaryotic parasites.

3. The method of claim 1, wherein the set of one or more reference genomes encompasses viral genomes selected from a taxon or taxa of one or more Families of viruses including Reoviridae, Caliciviridae, Flaviviridae, Orthomyxoviridae, Picornaviridae, Togaviridae, Paramyxoviridae, Bunyaviridae, Rhabdoviridae, Filoviridae, Coronaviridae, Astroviridae, Bornaviridae, Arteriviridae, Hepeviridae, and/or Retroviridae.

4. (canceled)

5. The method of claim 1, wherein the set of one or more reference genomes encompasses viral genomes selected from a taxon or taxa of one or more Genera of viruses including Ahjdlikevirus, Alfamovirus, Allexivirus, Allolevivirus, Alphabaculovirus, Alphacarmotetravirus, Alphacoronavirus, Alphaentomopoxvirus, Alphafusellovirus, Alphaguttavirus, Alphalipothrixvirus, Alphamesonivirus, Alphanecrovirus, Alphanodavirus, Alphanudivirus, Alphapapfflomavirud, Alphapartifivirus, Alphapermutotetravirus, Alpha retro virus, Alphasphaerolipovirus, Alphaspiravirus, Alphatorquevirus, Alphaturrivirus, Alphavirus, Amalgavirus, Ambidensovirus Amdoparvovirus, Ampelovirus, Ampullavirus, Andromedalikevirus, Anulavirus, Aparavirus, Aphthovirus, Apscaviroid, Aquabimavirus, Aqua ma virus, Aquaparamyxovirus, Aquareovirus, Arterivirus, Ascovirus, Asfivirus, Atadenovirus, Aureusvirus, Aurivirus Avastrovirus, Avenavirus, Aveparvovirus, Aviadenovirus, Avibimavirus, Avihepadnavirus, Avihepatovirus, Avipoxvirus, Avisivirus, Avsunviroid, Avulavirus, Bacifiamavirus, Babuvirus, Bacilladnavirus, Bamavirus, Badnavirus, Bafinivirus, Bcep22likevirus, Bamyardlikevirus, Batrachovirus, Bdellomicrovirus, Bcep78likevirus, Bcepmulikevirus, Benyvirus, Becurtovirus, Begomovirus, Betaentomopoxvirus, Betabaculovirus, Betacoronavirus, Betalipothrixvirus, Betafusellovirus, Beta gutta virus, Betanudivirus, Betanecrovirus, Betanodavirus, Betaretrovirus, Betapapillomavirus, Betapartitivirus, Betatorquevirus, Betasphaerolipovirus, Beta tetra virus, Bignuzlikevirus, Bicaudavirus, Bidensovirus, Bomavirus, Blosnavirus, Bocaparvovirus, Bracovirus, Botrexvirus, Bppunalikevirus, Bromovirus, Brambyvirus, Brevidensovirus, Bronlikevirus, Cafeteria virus, Bymovirus, Cardiovirus, C2likevirus, C5likevirus, Carmovirus, Capillovirus, Capripoxvirus, Cervidpoxvirus, Cardoreovirus, Carla virus, Che9clikevirus, Caulimovirus, Cavemovirus, Chipapillomavirus, Charlielikevirus, Che8likevirus, Chloro virus, Cheravirus, Chilikevirus, Circovirus, Chlamydiamicrovirus, Chloriridovirus, Clava virus, Chrysovirus, Cilevirus, Coccolithovirus, Citrivirus, Cjwunalikevirus, Comovirus, Closterovirus, Cocadviroid, Corticovirus, Coleviroid, Coltivirus, Cp8unalikevirus, Copiparvovirus, Comdoglikevirus, Crocodylidpoxvirus, Cosa virus, Cp220likevirus, Cuevavirus, Crinivirus, Cripavirus, Cyprinivirus, Cryspovirus, Cucumovirus, Cytorhabdovirus, Curtovirus, Cypovirus, Cysto virus, Cytomegalovirus, D3likevirus, Deltalipothrixvirus, Deltabaculovirus, D3112likevirus, Deltaretrovirus, Deltapapillomavirus, Deltacoronavirus, Dependoparvovirus, Deltatorquevirus, Deltapartitivirus, Dinodnavirus, Dianthovirus, Delta virus, Dyodeltapapillomavirus, Dinomavirus, Dicipivirus, Dyoiotapapillomavirus, Dyoepsilonpapillomavirus, Dinovemavirus, Dyomupapillomavirus, Dyokappapapillomavirus, Dyoetapapillomavirus, Dyopipapillomavirus, Dyonupapillomavirus, Dyolambdapapillomavirus, Dyothetapapillomavirus Dyorhopapillomavirus, Dyoomikronpapillomavirus, Dyoxipapillomavirus, Dyosigmapapillomavirus, Dyozetapapillomavirus, Emaravirus, Enterovirus, Ebola virus, Epsilon15likevirus, Enamovirus, Elaviroid, Epsilontorquevirus, Entomobimavirus, Endomavirus, Errantivirus, Epsilonpapillomavirus, Ephemerovirus, Etatorquevirus, Eragrovirus, Epsilonretrovirus, Fabavirus, Erythroparvovirus, Erbovirus, Fijivirus, Felixounalikevirus, Etapapillomavirus, Furovirus, Flavivirus, F116likevirus, Ferlavirus, Fovea virus, Gallivirus, Gammabaculovirus, Gammaentomopoxvirus, Gammalipothrixvirus, Gammapartitivirus, Gamma retro virus, Gallantivirus, Gammatorquevirus, Giardia virus, Gammacoronavirus, Glossina virus, Gyro virus, Gamma papilloma virus, Gammasphaerolipovirus, Globulovirus, Halolikevirus, Hantavirus, Hemi virus, Henipavirus, Hepandensovirus, Hepato virus, Hapunalikevirus, Hk578likevirus, Hordeivirus, Hepacivirus, Hpunalikevirus, Hunnivirus, Higrevirus, Hostuviroid, Hypovirus, Ichtadenovirus, 13likevirus, Idnoreovirus, Ictalurivirus, Ilarvirus, lebhlikevirus, Ichnovirus, Influenzavirus B, Iltovirus, Idaeovirus, Iotapapillomavirus, Influenzavirus C, Iflavirus, Iridovirus, lotatorquevirus, Influenzavirus A, Inovirus, Ipomovirus, Isavirus, Iteradensovirus, Jerseylikevirus, Kappapapillomavirus, Kappatorquevirus, Kobuvirus, Kunsagivirus, Labymavirus, Lambdapapillomavirus, Lagovirus, Lentivirus, Lambdatorquevirus, L5likevirus, Lola virus, Leporipoxvirus, Lambdalikevirus, Lymphocryptovirus, Luteovirus, Leishmania virus, Lymphocystivirus, Levi virus, Luz24likevirus, Lyssavirus, Machlomovirus, Mamastrovirus, Macanavirus, Marafivirus, Macluravirus, Marna virus, Mammarenavirus, Macavirus, Mastrevirus, Marburg virus, Macula virus, Megrivirus, Marseille virus, Mandarivirus, Micro virus, Megabimavirus, Mardivirus, Mischivirus, Metapneumovirus, Mastadenovirus, Morbillivirus, Mimi virus, Megalocytivirus, Mupapillomavirus, Mitovirus, Meta virus, Mycoflexivirus, Mosavirus, Mimoreovirus, Muromegalovirus, Molluscipoxvirus, Mycoreovirus, Mulikevirus, Musca virus, N15likevirus, Nanovirus, Nepovirus, N4likevirus, Nucleorhabdovirus, Namavirus, Nairovirus, Norovirus, Nebovirus, Nupapillomavirus, Novirhabdovirus, Nyavirus, Omegalikevirus, Omikronpapillomavirus, Orthobunyavirus, Oka virus, Orthopoxvirus, Omega papilloma virus, Oleavirus, Oscivirus, Ophiovirus, Omegatetravirus, Orthohepadnavirus, Orbivirus, Orthoreovirus, Orthohepevirus, Ostreavirus, Oryzavirus, Ourmiavirus, Parechovirus, Pbiunalikevirus, Pegivirus, P2likevirus, P22likevirus, Percavirus, Panicovirus, P23likevirus, Petuvirus, Pasivirus, Parapoxvirus, Phi29likevirus, Pbunalikevirus, Passerivirus, Phicd119likevirus, Pelamoviroid, Peduvirus, Phietalikevirus, Perhabdovirus, Penstyldensovirus, Phrjlunalikevirus, Pgonelikevirus, Pestivirus, Phipapillomavirus, Phic3unalikevirus, Phaeovirus, Picobimavirus, Phie125likevirus, Phicbklikevirus, Plasma virus, Phifllikevirus, Phieco32likevirus, Poacevirus, Phikmvlikevirus, Phihlikevirus, Polyomavirus, Phlebovirus, Phikzlikevirus, Potexvirus, Pipapillomavirus, Phytoreovirus, Proboscivirus, Plectrovirus, Piscihepevirus, Pseudovirus, Polemovirus, Pneumovirus, Punalikevirus, Pomovirus, Polerovirus, Potyvirus, Pospiviroid, Protoparvovirus, Prasinovirus, Prymnesiovirus, Psimunalikevirus, Psipapillomavirus, Quaranjavirus, Quadrivirus, Raphidovirus, Reylikevirus, Rhopapillomavirus, Reptarenavirus, Roseolovirus, Rhadinovirus, Rubulavirus, Rosadnavirus, Rana virus, Rotavirus, Respirovirus, Rubivirus, Rudivirus, Rymovirus, Rhizidiovirus, Rosa virus, Salivirus, Sap6likevirus, Schizot4virus, Sadwavirus, Seadomavirus, Salmonivirus, Sequivirus, Sapelovirus, Siadenovirus, Sclerodamavirus, Sakobuvirus, Sigma virus, Semotivirus, Salterprovirus, Skunalikevirus, Sfi1unalikevirus, Sapovirus, Soymovirus, Sicinivirus, Scuta virus, Spiromicrovirus, Simplexvirus, Seneca virus, Spumavirus, Sobemovirus, Sfi21dtunalikevirus, Sp6likevirus, Sigmapapillomavirus, Spounalikevirus, Sire virus, Suipoxvirus, Solendovirus, Spbetalikevirus, Sprivivirus, T4 virus, Taupapillomavirus, Tepovirus, Thetapapillomavirus, T5likevirus, Tibrovirus, Tectivirus, Tobravirus, Teschovirus, T7likevirus, Toro virus, Thetatorquevirus, Tenuivirus, Totivirus, Tm4likevirus, Tetraparvovirus, Trichomonasvirus, Tombusvirus, Thogotovirus, Tunalikevirus, Torradovirus, Tobamovirus, Tumcurtovirus, Tp2unalikevirus, Topocuvirus, Trichovirus, Tospovirus, Tungrovirus, Tremovirus, Twortlikevirus, Tritimovirus, Tupavirus, Tymovirus, Umbra virus, Upsilonpapillomavirus, Vancellovirus, Velarivirus, Vesiculovirus, Victorivirus, Vitivirus, Varicosavirus, Vesivirus, Viunalikevirus, Waikavirus, Wbetalikevirus, Whispovirus, Xp10likevirus, Xipapillomavirus, Yualikevirus, Yatapoxvirus, Zetapapillomavirus, Zetatorquevirus, and/or Zea virus.

6. The method of claim 1, wherein the set of one or more reference genomes encompasses viral genomes from a taxon or taxa of one or more Species of viruses including West Nile Virus, dengue virus, tick-borne encephalitis virus, Japanese encephalitis virus, yellow fever virus, Zika virus, cell fusing agent virus, Palm Creek virus and/or Parramatta River virus.

7. The method of claim 1, wherein the set of one or more reference genomes encompasses bacterial genomes from one or more from a taxon or taxa of one or more Genera of bacteria including Heliobacter, Aerobacter, Rhizobium, Agrobacterium, Bacillus, Clostridium, Pseudomonas, Xanthomonas, Nitrobacteriaceae, Nitrobacter, Nitrosomonas, Thiobacillus, Spirillum, Vibrio, Bacteroides, Corynebacterium, Listeria, Escherichia, Klebsiella, Salmonella, Serratia, Shigella, Erwinia, Rickettsia, Chlamydia, Mycoplasma, Actinomyces, Streptomyces, Mycobacterium, Polyangium, Micrococcus, Staphylococcus, Lactobacillus, Diplococcus, Streptococcus, and/or Campylobacter.

8. The method of claim 1, wherein the set of one or more reference genomes encompasses fungal genomes from one or more from a taxon or taxa of one or more Genera of fungi including Anaeromyces, Caecomyces, Allomyces, Entyloma, Diskagma, Blastocladia, Funneliformis, Entylomella, Coelomomyces, Glomus (fungus), Fusidium, Heptameria, Holmiella, Homostegia, Hyalocrea, Hyalosphaera, Hypholoma, Hypobryon, Hysteropsis, Koordersiella, Karschia, Kirschsteiniothelia, Lembosiopeltis, Kullhemia, Kusanobotrys, Leptodothiorella, Lanatosphaera, Lasiodiplodia, Leveilliina, Lepidopterella, Lepidostroma, Lollipopaia, Leptosphaerulina, Leptospora, Macrovalsaria, Lichenostigma, Licopolia, Massariola, Lopholeptosphaeria, Maireella, Microdothella, Macroventuria, Microcyclella, Mycoglaena, Melanodothis, Montagnella, Mycoporopsis, Moniliella, Mycopepon, Myriangium, Mycomicrothefia, Mycothyridium, Mytilostoma, Mycosphaerella, Myfifinidion, Neofusicoccum, Myriosfigmella, Neocallimasfix, Oomyces, Neopeckia, Orpinomyces, Ostreichnion, Ophiosphaerella, Paropodia, Passeriniella, Passerinula, Pedumispora, Peyronellaea, Phaeoacremonium, Phaeocyrfidula, Phaeoglaena, Phaeopeltosphaeria, Phaeoramularia, Phaeosperma, Phaneromyces, Phialophora, Philonectria, Phragmocapnias, Phragmosperma, Piedraia, Piromyces, Placocrea, Placostromella, Plagiostromella, Plejobolus, Pleostigma, Polychaeton, Pseudocercospora, Pseudocryptosporella, Pseudogymnoascus, Pseudothis, Pycnocarpon, Rhytidhysteron, Rhizophagus (fungus), Rhopographus, Roselfinula, Rhytisma, Robillardiella, Roussoellopsis, Rosenscheldia, Rostafinskia, Sarcopodium, Savulescua, Saksenaeaceae, Scolecobonaria, Scoficotrichum, Schizoparme, Semifissispora, Septoria, Scorias, Sphaceloma, Sphaerellothecium, Spathularia, Stagonosporopsis, Stenella (fungus), Sphaerulina, Stigmina (fungus), Stioclettia, Stigmidium, Sydowia, Tephromela, Stuartella, Teichosporella, Thalloloma, Taeniolella, Thalassoascus, Togninia, Teratosphaeria, Thyrospora, Thyridaria, Yarrowia, Wettsteinina, Valsaria, Ustilaginoidea, Yoshinagella, Wemerella (fungus), and/or Vismya.

9. The method of claim 1, wherein the set of one or more reference genomes encompasses genomes from one or more from a taxon or taxa of pathogenic microorganisms that are resistant to a particular anti-pathogen treatment.

10. The method of claim 9, wherein the anti-pathogen therapy is selected from an antibiotic treatment, antiviral treatment, antifungal treatment, or algicide.

11. The method of claim 1, wherein the MSA-aligned genomes are partitioned into overlapping 500 nt to 600 nt segments with a 200 nt to 300 nt overlap.

12. The method of claim 1, wherein the candidate primer sequences are 13 bp to 15 bp in length.

13. The method of claim 1, wherein the forward or reverse candidate primer sequences are selected from 40 nt to 60 nt regions at the ends of each segment.

14. The method of claim 1, further comprising the step of:
(ix) chemically synthesizing a set of spiked primers that corresponds with the set of spiked primer sequences.

15. (canceled)

16. A method of detecting a first taxon or taxa of pathogenic microorganisms in a sample, the method comprising:
applying a sequencing assay to the sample to obtain sequence reads, the sequencing assay including the set of spiked primers of claim 14 and random primers; and
analyzing the sequence reads to determine whether the first taxon of pathogenic microorganisms and/or one or more other taxa of pathogenic microorganisms are present in the sample.

17. The method of claim 16, wherein the sample is from a subject.

18. (canceled)

19. The method of claim 16, wherein the sample is selected from whole blood, serum, plasma, urine, tissue sample, biopsy sample, isolated DNA and isolated RNA.

20. (canceled)

21. The method of claim 16, wherein the sample is obtained from an environmental site believed to be infected or contaminated by a taxon or taxa of pathogenic microorganisms.

22. (canceled)

23. The method of claim 16, wherein the sample is from a vector that is known to transmit pathogenic microorganisms, wherein the vector is selected from the group consisting of a mosquito, sandfly, tick, triatomine bug, tsetse fly, flea, black fly, aquatic snail, and lice.

24. (canceled)

25. The method of claim 16, wherein the sequencing assay comprises or utilizes polymerase chain reaction (PCR).

26. (canceled)

27. The method of claim 16, wherein the sequencing assay comprises reverse transcription of a sample containing RNA using any of the primers set forth in SEQ ID NOs: 1-96, 399-1562, 1563-3553, and 3554-7324.

28. The method of claim 16, wherein the sequencing assay provides greater than 10 sequencing reads and fewer than 100,000 sequencing reads per amplified target nucleic acid.

29. The method of claim 16, wherein at least one, two, three, four, or more of the sequence regions targeted by the spiked primers were identified in the set of one or more reference sequences corresponding to the taxon or taxa of pathogenic microorganisms.

30. The method of claim 16, wherein the taxon or taxa of pathogenic microorganisms is present in the sample at a volume of less than 1,000 genome copies per mL.

31. (canceled)

32. The method of claim 16, wherein the sample comprises a different taxon or taxa of pathogenic microorganisms at a volume of between 10,000-100,000 genome copies per mL.

33. The method of claim 16, wherein at least one of the set of spiked primers of the sequencing assay comprises a nucleotide sequence selected from SEQ ID NOs:1-96 or 399-7324.

34. The method of claim 33, wherein the set of spiked primers of the sequencing assay comprises primers having nucleotide sequences of SEQ ID NOs:1-96.

35. The method of claim 33, wherein the set of spiked primers of the sequencing assay comprise primers having nucleotide sequences of SEQ ID NOs: 399-1562.

36. The method of claim 33, wherein the set of spiked primers of the sequencing assay comprise primers having nucleotide sequences of SEQ ID NOs: 1563-3553.

37. The method of claim 33, wherein the set of spiked primers of the sequencing assay comprise primers having nucleotide sequences of SEQ ID NOs: 3554-7324.

38. The method of claim 33, wherein the spiked primers further comprise an adaptor sequence.

39. The method of claim 38, wherein the adapter sequence is positioned 5′ of the spiked primer sequences and comprises the sequence of SEQ ID NO:97.

40. The method of claim 16, wherein the random primers are random hexamers, random septamers, random octamers, and/or random nonamers.

41. (canceled)

42. The method of claim 16, wherein the ratio of spiked primers to random primers in the sequencing assay is 2:1, 3:1, 4:1, 5:1, 6:1, 7:1, 8:1, 9:1, 10:1, or greater than 10:1.

43. (canceled)

44. The method of claim 16, wherein the sequencing assay further comprises a probe that is used to determine the amount of amplified product produced in the sequencing assay.

45. A kit comprising a set of spiked primers that comprises primers having nucleotide sequences of SEQ ID NOs:1-96, SEQ ID NOs: 399-1562, SEQ ID NOs: 1563-3553, and/or SEQ ID NOs: 3554-7324.

46. The kit of claim 45, wherein the primers further comprise an adapter sequence.

47. The kit of claim 46, wherein the adapter sequence is positioned 5′ of the primer sequences and comprises the sequence of SEQ ID NO:97.

48. The kit of claim 45, wherein the kit further comprises random hexamer and/or random nonamer primers.

49. The kit of claim 45, wherein the kit further comprises one or more probes having sequences selected from SEQ ID NOs:98-398.
